(19) United States
(12) Patent Application Publication
(10) Pub. No.: US 2009/0082341 A1
(43) Pub. Date: Mar. 26, 2009

(54) 4-hydroxy-4-methyl-piperidine-1-carboxylic acid (4-methoxy-7-morpholin-4-yl-benzothiazol-2-yl)-amide FOR THE TREATMENT OF POST-TRAUMATIC STRESS DISORDER

(75) Inventors: Tom Woiwode, South San Francisco, CA (US); Mark Moran, South San Francisco, CA (US)

Correspondence Address: FENWICK & WEST LLP, SILICON VALLEY CENTER, 801 CALIFORNIA STREET, MOUNTAIN VIEW, CA, 94041, US

(73) Assignee: SYNOSIA THERAPEUTICS, South San Francisco, CA (US)

(21) Appl. No.: 12/178,509

(22) Filed: Jul. 23, 2008

Related U.S. Application Data
(60) Provisional application No. 60/935,035, filed on Jul. 23, 2007.

(51) Int. Cl. A61K 31/5377; A61K 31/551; A61P 25/22

(52) U.S. Cl. 514/221; 514/233.8

(57) ABSTRACT

Provided are methods of treating post-traumatic stress disorder with 4-hydroxy-4-methyl-piperidine-1-carboxylic acid (4-methoxy-7-morpholin-4-yl-benzothiazol-2-yl)-amide. Also provided are methods of improving resilience with 4-hydroxy-4-methyl-piperidine-1-carboxylic acid (4-methoxy-7-morpholin-4-yl-benzothiazol-2-yl)-amide. Also provided are methods of diagnosing post-traumatic stress disorder in a patient.

### CROSS-REFERENCE TO RELATED APPLICATIONS

This application claims the benefit of and priority under 35 U.S.C. § 119(e) to U.S. Provisional Patent Application Ser. No. 60/935,035, “4-hydroxy-4-methyl-piperidine-1-carboxylic acid (4-methoxy-7-morpholin-4-yl-benzothiazol-2-yl)-amide FOR THE TREATMENT OF POST-TRAUMATIC STRESS DISORDER” filed Jul. 23, 2007, which is incorporated herein by reference in its entirety.

### FIELD OF INVENTION

This relates generally to methods for treating post-traumatic stress disorder and more particularly methods of treating post-traumatic stress disorder with 4-hydroxy-4-methyl-piperidine-1-carboxylic acid (4-methoxy-7-morpholin-4-yl-benzothiazol-2-yl)-amide. Also provided are methods of improving resilience with 4-hydroxy-4-methyl-piperidine-1-carboxylic acid (4-methoxy-7-morpholin-4-yl-benzothiazol-2-yl)-amide. Also provided are methods of diagnosing post-traumatic stress disorder in a patient, among other things.

### BACKGROUND OF THE INVENTION

Anxiety disorders are the most commonly occurring disorders of the psychiatric illnesses with an immense economic burden. In addition to generalized anxiety disorder, they encompass post-traumatic stress disorder, panic disorder, obsessive compulsive disorder and social as well as other phobias.

Post-traumatic stress disorder can be severe and chronic, with some studies suggesting a lifetime prevalence of 1.3% to 7.8% in the general population. Post-traumatic stress disorder typically follows a psychologically distressing traumatic event. These events may include military combat, terrorist incidents, physical assault, sexual assault, motor vehicle accidents, and natural disasters, for example. The response to the event can involve intense fear, helplessness, or horror. Most people recover from the traumatic event with time and return to normal life. In contrast, in post-traumatic stress disorder victims, symptoms persist and may worsen with time, preventing a return to normal life.

Psychotherapy is currently the backbone of post-traumatic disorder treatment. Methods include cognitive-behavioral therapy, exposure therapy, and eye movement desensitization and reprocessing. Medication can enhance the effectiveness of psychotherapy. Selective serotonin reuptake inhibitors (SSRIs), such as sertraline (Zoloft®) and paroxetine (Paxil®), are the only medications approved for treating PTSD by the Food and Drug Administration. Many unwanted side effects and characteristics are associated with SSRI usage. These include concerns about drug interactions, gastrointestinal side effects, sexual side effects, suicidal ideation, acute anxiogenic effects, and slow onset of action. Some tricyclic antidepressants (TCAs) and monamine oxidase inhibitors (MAOIs) appear to have some efficacy but patient tolerance is low due to the high incidence of side effects. MAOIs have dietary restriction requirements and are linked to hypertensive events. TCAs have anticholinergic and cardiovascular side effects. Lamotrigine, a sodium channel blocker, has had some efficacy in treating post-traumatic stress disorder in a small scale placebo controlled study. Difficulty in the use of lamotrigine due the to necessity for titration and the risk of developing Steven Johnson Syndrome, a life threatening rash, render it a poor candidate for therapeutic use.

There is a need for the development of treatments for post-traumatic stress disorder that are safe and effective.

Adenosine and its receptors have multiple functions in the modulation of central nervous system activities. The action of adenosine as a neurotransmitter is mediated through adenosine receptors belonging to the family of G protein-coupled receptors. There are four adenosine receptors, A₁, A_{2A}, A_{2B}, A₃, known thus far. Activation of adenosine receptors by adenosine initiates signal transduction mechanisms. Each of the adenosine receptor subtypes has been classically characterized by the adenylate cyclase effector system, which utilizes cyclic adenomonophosphate (cAMP) as a second messenger. The A1 and A3 receptors couple with Gi proteins and inhibit adenylate cyclase, leading to a decrease in cellular cAMP levels, while A_{2A} and A_{2B} receptors couple to Gs proteins and activate adenylate cyclase, leading to an increase in cellular cAMP levels.

A₁ receptors are widely distributed in the brain, while the distributions of A_{2B} and A₃ receptors are less clear. A_{2A} receptors are highly abundant in discrete brain regions, such as the striatum, nucleus accumbens and olfactory tubercles, which is consistent with the proposed role of these receptors in modulating neurotransmission. These regions of the brain are involved in the control of emotion, reward and pleasure and are, therefore, centrally located to modulate the conversion of motivation into action. Adenosine signaling may also indirectly modulate dopaminergic signaling. 4-hydroxy-4-methyl-piperidine-1-carboxylic acid (4-methoxy-7-morpholin-4-yl-benzothiazol-2-yl)-amide is an A_{2A} receptor antagonist.

### SUMMARY OF THE INVENTION

Provided are methods of treating a patient diagnosed with post-traumatic stress disorder. The methods include administering to the patient a therapeutically effective amount of 4-hydroxy-4-methyl-piperidine-1-carboxylic acid (4-methoxy-7-morpholin-4-yl-benzothiazol-2-yl)-amide.

Also provided are methods of treating post-traumatic stress disorder in a patient. The methods include diagnosing the patient with post-traumatic stress disorder; administering to the patient a therapeutically effective amount of 4-hydroxy-4-methyl-piperidine-1-carboxylic acid (4-methoxy-7-morpholin-4-yl-benzothiazol-2-yl)-amide; assessing at least one of sign, symptom, and symptom cluster of post-traumatic stress disorder; and determining that the post-traumatic stress syndrome is improved if the 4-hydroxy-4-methyl-piperidine-1-carboxylic acid (4-methoxy-7-morpholin-4-yl-benzothiazol-2-yl)-amide reduces at least one of sign, symptom, and symptom cluster of post-traumatic stress disorder.

Also provided are methods of improving resilience in a patient. The methods include administering a therapeutically effective amount of 4-hydroxy-4-methyl-piperidine-1-carboxylic acid (4-methoxy-7-morpholin-4-yl-benzothiazol-2-yl)-amide.

Also provided are methods of diagnosing post-traumatic stress disorder in a patient. The methods include administering to the patient a therapeutically effective amount of 4-hydroxy-4-methyl-piperidine-1-carboxylic acid (4-methoxy-7-morpholin-4-yl-benzothiazol-2-yl)-amide and assessing at least one of sign, symptom, or symptom cluster of post-traumatic stress disorder; and diagnosing post-traumatic stress disorder in the patient if the 4-hydroxy-4-methyl-piperidine-1-carboxylic acid (4-methoxy-7-morpholin-4-yl-benzothiazol-2-yl)-amide reduces at least one of sign, symptom, and symptom cluster of post-traumatic stress disorder. In certain embodiments the patient is a child, adolescent, or adult.

### DESCRIPTION OF DRAWINGS

FIG. 1 Shows Reversal of APEC-Induced Hypolocomotion in Rats

FIG. 2 Shows the Results of 4-hydroxy-4-methyl-piperidine-1-carboxylic acid (4-methoxy-7-morpholin-4-yl-benzothiazol-2-yl)-amide in Swim Stress Test in Rats

FIG. 3 Shows the Reversal of Stress-Induced Anhedonia in Rats by 4-hydroxy-4-methyl-piperidine-1-carboxylic acid (4-methoxy-7-morpholin-4-yl-benzothiazol-2-yl)-amide

FIG. 4 Differential-Reinforcement-of-Low-Rate (30 Seconds) Test in Rats

FIG. 5 Shows Results of Elevated Plus-Maze Test in Rats

FIG. 6 Shows Results of Passive Avoidance Test in Rats

FIG. 7 Shows the structure of 4-hydroxy-4-methyl-piperidine-1-carboxylic acid (4-methoxy-7-morpholin-4-yl-benzothiazol-2-yl)-amide

FIG. 8 Shows Radioligand Binding Assay Conditions

FIG. 9 Shows the Affinity of 4-hydroxy-4-methyl-piperidine-1-carboxylic acid (4-methoxy-7-morpholin-4-yl-benzothiazol-2-yl)-amide at A1, A2A, A2B, and A3 receptors in Various Animal Species.

FIG. 10 Shows raw data, curves and data calculation for adenosine receptor Ca2+ Flux Of Adenosine human A1-Gα16 receptor in CHO cells in FLIPR (1)

FIG. 11 Shows raw data, curves, and data calculations for Adenosine receptor Ca2+ Flux of Adenosine human A1-Gα16 receptor in CHO cells in FLIPR (2)

FIG. 12 Shows raw data, curves, and data calculations for Adenosine receptor Ca2+ Flux of Adenosine human A1-Gα16 receptor in CHO cells in FLIPR (3)

FIG. 13 Shows raw data, curves, and data calculations for Adenosine receptor Ca2+ Flux of Adenosine human A1-Gα16 receptor in CHO cells in FLIPR (4)

FIG. 14 Shows raw data, curves, and data calculations for Adenosine receptor Ca2+ Flux of Adenosine human A1-Gα16 receptor in CHO cells in FLIPR (5)

FIG. 15 Shows raw data, curves, and data calculations for Adenosine receptor Ca2+ Flux of Adenosine human A1-Gα16 receptor in CHO cells in FLIPR (6)

FIG. 16 Shows raw data, curves, and data calculations for Adenosine receptor Ca2+ Flux of Adenosine human A2A-Gα16 receptor in CHO cells in FLIPR (1)

FIG. 17 Shows raw data, curves, and data calculations for Adenosine receptor Ca2+ Flux of Adenosine human A2A-Gα16 receptor in CHO cells in FLIPR (2)

FIG. 18 Shows raw data, curves, and data calculations for Adenosine receptor Ca2+ Flux of Adenosine human A2A-Gα16 receptor in CHO cells in FLIPR (3)

FIG. 19 Shows raw data, curves, and data calculations for Adenosine receptor Ca2+ Flux of Adenosine human A2A-Gα16 receptor in CHO cells in FLIPR (4)

FIG. 20 Shows raw data, curves, and data calculations for Adenosine receptor Ca2+ Flux of Adenosine human A2A-Gα16 receptor in CHO cells in FLIPR (5)

FIG. 21 Shows raw data, curves, and data calculations for Adenosine receptor C2+ Flux of Adenosine human A2A-Gα16 receptor in CHO cells in FLIPR (6)

FIG. 22 Shows General Procedures of Binding Assays

FIG. 23 Shows General Procedures of Binding Assays

FIG. 24 Shows General Procedures of Binding Assays

FIG. 25 Shows General Procedures of Binding Assays

FIG. 26 Shows Experimental Conditions

FIG. 27 Shows Experimental Conditions

FIG. 28 Shows Experimental Conditions

FIG. 29 Shows Experimental Conditions

FIG. 30 Shows Experimental Conditions

FIG. 31 Shows Experimental Conditions

FIG. 32 Shows Experimental Conditions

FIG. 33 General Procedures for Enzyme Assays

FIG. 34 Experimental Conditions for Enzyme Assays

FIG. 35 Experimental Conditions for Enzyme Assays

FIG. 36 Experimental Conditions for Enzyme Assays

FIG. 37 Shows Mean Values for the Effects of 4-hydroxy-4-methyl-piperidine-1-carboxylic acid (4-methoxy-7-morpholin-4-yl-benzothiazol-2-yl)-amide in Binding Assays

FIG. 38 Shows Mean Values for the Effects of 4-hydroxy-4-methyl-piperidine-1-carboxylic acid (4-methoxy-7-morpholin-4-yl-benzothiazol-2-yl)-amide in Binding Assays

FIG. 39 Shows Mean Values for the Effects of 4-hydroxy-4-methyl-piperidine-1-carboxylic acid (4-methoxy-7-morpholin-4-yl-benzothiazol-2-yl)-amide in Binding Assays

FIG. 40 Shows Mean Values for the Effects of 4-hydroxy-4-methyl-piperidine-1-carboxylic acid (4-methoxy-7-morpholin-4-yl-benzothiazol-2-yl)-amide in Binding Assays

FIG. 41 Shows Mean Values for the Effects of 4-hydroxy-4-methyl-piperidine-1-carboxylic acid (4-methoxy-7-morpholin-4-yl-benzothiazol-2-yl)-amide in Binding Assays

FIG. 42 Shows Mean Values for the Effects of 4-hydroxy-4-methyl-piperidine-1-carboxylic acid (4-methoxy-7-morpholin-4-yl-benzothiazol-2-yl)-amide in Binding Assays

FIG. 43 Shows Mean Values for the Effects of 4-hydroxy-4-methyl-piperidine-1-carboxylic acid (4-methoxy-7-morpholin-4-yl-benzothiazol-2-yl)-amide in Binding Assays

FIG. 44 Shows IC50 and Ki Values for Binding Assays

FIG. 45 Shows IC50 and Ki Values for Binding Assays

FIG. 46 Shows IC50 and Ki Values for Binding Assays

FIG. 47 Shows IC50 and Ki Values for Binding Assays

FIG. 48 Shows IC50 and Ki Values for Binding Assays

FIG. 49 Shows Mean Values for Effects of 4-hydroxy-4-methyl-piperidine-1-carboxylic acid (4-methoxy-7-morpholin-4-yl-benzothiazol-2-yl)-amide in Enzyme Assays

FIG. 50 Shows Mean Values for Effects of 4-hydroxy-4-methyl-piperidine-1-carboxylic acid (4-methoxy-7-morpholin-4-yl-benzothiazol-2-yl)-amide in Enzyme Assays

FIG. 51 Shows IC50 and EC50 Values for Each Reference Compound

FIG. 52 Shows IC50 and EC50 Values for Each Reference Compound

FIG. 53 Shows IC50 and EC50 Values for Each Reference Compound

FIG. 54 Shows IC50 and EC50 Values for Each Reference Compound

FIG. 55 Shows 4-hydroxy-4-methyl-piperidine-1-carboxylic acid (4-methoxy-7-morpholin-4-yl-benzothiazol-2-yl)-amide significantly and dose-dependently reversed APEC-induced deficits in locomotor activity compared with controls, with ID50 and ID90 values of 0.5 mg/kg and 3.4 mg/kg, respectively.

FIG. 56 Shows Oral administration of 4-hydroxy-4-methyl-piperidine-1-carboxylic acid (4-methoxy-7-morpholin-4-yl-benzothiazol-2-yl)-amide to female rats significantly and dose-dependently decreased the mean total duration of immobility compared with controls. A similar result was obtained with desipramine (100 mg/kg p.o.), the tricyclic antidepressant used as a reference drug.

FIG. 57 Shows Anhedonia Index Against Stress Period

FIG. 58 Shows Differential-Reinforcement-of-Low-Rate (30 Seconds) Test in Rats

FIG. 59 Shows Correct Responses Among Treatment Groups

FIG. 60 shows the mean (+/−SEM) time spent in open arms (a), number of entries into the open arms (b), distance traveled in open arms (c) and distance traveled per second in closed arms (d) of either chlordiazepoxide (10 mg/kg po), vehicle or 4-hydroxy-4-methyl-piperidine-1-carboxylic acid (4-methoxy-7-morpholin-4-yl-benzothiazol-2-yl)-amide (doses 3, 10 and 30 mg/kg, po)-treated animals. Statistical analysis, Dunnett's test: * p<0.05 ** p<0.01 versus vehicle and (-test: # p<0.05 ## p<0.01 ### p<0.001 versus vehicle.

FIG. 61 shows the effect on time in open arms (sec) of 10 mg/kg chlordiazepoxide (CDZ 10), vehicle (veh) and 4-hydroxy-4-methyl-piperidine-1-carboxylic acid (4-methoxy-7-morpholin-4-yl-benzothiazol-2-yl)-amide at doses 3, 10, and 30 mg/kg in the Elevated Plus-maze.

FIG. 62 shows the effect on open arm entries of 10 mg/kg chlordiazepoxide (CDZ 10), vehicle (veh) and 4-hydroxy-4-methyl-piperidine-1-carboxylic acid (4-methoxy-7-morpholin-4-yl-benzothiazol-2-yl)-amide at doses 3, 10, and 30 mg/kg in the Elevated Plus-maze.

FIG. 63 shows the distance traveled in open arms (cm) of 10 mg/kg chlordiazepoxide (CDZ 10), vehicle (veh) and 4-hydroxy-4-methyl-piperidine-1-carboxylic acid (4-methoxy-7-morpholin-4-yl-benzothiazol-2-yl)-amide at doses 3, 10, and 30 mg/kg in the Elevated Plus-maze.

FIG. 64 shows the effect on speed in closed arms (cm/s) of 10 mg/kg chlordiazepoxide (CDZ 10), vehicle (veh) and 4-hydroxy-4-methyl-piperidine-1-carboxylic acid (4-methoxy-7-morpholin-4-yl-benzothiazol-2-yl)-amide at doses 3, 10, and 30 mg/kg in the Elevated Plus-maze.

### DETAILED DESCRIPTION

As used herein, the following words and phrases are generally intended to have the meanings as set forth below, except to the extent that the context in which they are used indicates otherwise.

As used herein “4-hydroxy-4-methyl-piperidine-1-carboxylic acid (4-methoxy-7-morpholin-4-yl-benzothiazol-2-yl)-amide” includes 4-hydroxy-4-methyl-piperidine-1-carboxylic acid (4-methoxy-7-morpholin-4-yl-benzothiazol-2-yl)-amide, as well as pharmaceutically acceptable salts thereof.

“Pharmaceutically acceptable salts” include, but are not limited to salts with inorganic acids, such as hydrochlorate, phosphate, diphosphate, hydrobromate, sulfate, sulfinate, nitrate, and like salts; as well as salts with an organic acid, such as malate, maleate, fumarate, tartrate, succinate, citrate, acetate, lactate, methanesulfonate, p-toluenesulfonate, 2-hydroxyethylsulfonate, benzoate, salicylate, stearate, and alkanoate such as acetate, HOOC—(CH2)n-COOH where n is 0-4, and like salts.

In addition, if a compound is obtained as an acid addition salt, the free base can be obtained by basifying a solution of the acid salt. Conversely, if the product is a free base, an addition salt, particularly a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used to prepare non-toxic pharmaceutically acceptable addition salts.

As used herein, the term “treating” refers to any manner in which at least one sign, symptom, or symptom cluster of a disease or disorder is beneficially altered so as to prevent or delay the onset, reduce the incidence or frequency, reduce the severity or intensity, retard the progression, prevent relapse, or ameliorate the symptoms or associated symptoms of the disease or disorder. For example, in post-traumatic stress disorder, treating the disorder can, in certain embodiments, cause a reduction in at least one of the frequency and intensity of at least one of a sign, symptom, and symptom cluster of post-traumatic stress disorder.

As used herein the phrase “diagnosed with post-traumatic stress disorder (PTSD)” refers to having a diagnosis of at least one sign, symptom, or symptom cluster indicative of post-traumatic stress disorder, a psychiatric disorder triggered by a traumatic event. Non-limiting examples of such traumatic events include military combat, terrorist incidents, physical assault, sexual assault, motor vehicle accidents, and natural disasters.

The Diagnostic and Statistical Manual of Mental Disorders-IV-Text revised (DSM-IV-TR), a handbook for mental health professionals that lists categories of mental disorders and the criteria, classifies post-traumatic stress disorder as an anxiety disorder. According to the DSM-IV-TR, a PTSD diagnosis can be made if:

1. the patient experienced, witnessed, or was confronted with an event or events that involved actual or threatened death or serious injury, or a threat to the physical integrity of self or others and the response involved intense fear, helplessness, or horror;

2. as a consequence of the traumatic event, the patient experiences at least 1 re-experiencing/intrusion symptom, 3 avoidance/numbing symptoms, and 2 hyperarousal symptoms, and the duration of the symptoms is for more than 1 month; and

3. the symptoms cause clinically significant distress or impairment in social, occupational, or other important areas of functioning.

In certain embodiments, if the patient's disorder fulfills DSM-IV-TR criteria, the patient is diagnosed with post-traumatic stress disorder. In certain embodiments, if the patient has at least one sign, symptom, or symptom cluster of post-traumatic stress disorder, the patient is diagnosed with post-traumatic stress disorder. In certain embodiments, a scale is used to measure a sign, symptom, or symptom cluster of post-traumatic stress disorder, and post-traumatic stress disorder is diagnosed on the basis of the measurement using that scale. In certain embodiments, a “score” on a scale is used to diagnose or assess a sign, symptom, or symptom cluster of post-traumatic stress disorder. In certain embodiments, a “score” can measure at least one of the frequency, intensity, or severity of a sign, symptom, or symptom cluster of post-traumatic stress disorder.

As used herein, the term “scale” refers to a method to measure at least one sign, symptom, or symptom cluster of post-traumatic stress disorder in a patient. In certain embodiments, a scale may be an interview or a questionnaire. Non-limiting examples of scales are Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

As used herein, the terms “sign” and “signs” refer to objective findings of a disorder. In certain embodiments, a sign can be a physiological manifestation or reaction of a disorder. In certain embodiments, a sign may refer to heart rate and rhythm, body temperature, pattern and rate of respiration, blood pressure. In certain embodiments, signs can be associated with symptoms. In certain embodiments, signs can be indicative of symptoms.

As used herein, the term “symptom” and “symptoms” refer to subjective indications that characterize a disorder. Symptoms of post-traumatic stress disorder may refer to, for example, but not limited to recurrent and intrusive trauma recollections, recurrent and distressing dreams of the traumatic event, acting or feeling as if the traumatic event were recurring, distress when exposed to trauma reminders, physiological reactivity when exposed to trauma reminders, efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, anxiety with unfamiliar surroundings, difficulty falling or staying asleep, irritability or outbursts of anger, difficulty concentrating, hypervigilance, and exaggerated startle response. In certain embodiments, potentially threatening stimuli can cause hyperarousal or anxiety. In certain embodiments, the physiological reactivity manifests in at least one of abnormal respiration, abnormal cardiac rate of rhythm, abnormal blood pressure, abnormal function of a special sense, and abnormal function of sensory organ. In certain embodiments, restricted range of effect characterized by diminished or restricted range or intensity of feelings or display of feelings can occur and a sense of a foreshortened future can manifest in thinking that one will not have a career, marriage, children, or a normal life span. In certain embodiments, children and adolescents may have symptoms of post-traumatic stress disorder such as, for example and without limitation, disorganized or agitated behavior, repetitive play that expresses aspects of the trauma, frightening dreams which lack recognizable content, and trauma-specific reenactment.

As used herein, the term “symptom cluster” refers to a set of signs, symptoms, or a set of signs and symptoms, that are grouped together because of their relationship to each other or their simultaneous occurrence. For example, in certain embodiments post-traumatic stress disorder is characterized by three symptom clusters: re-experiencing/intrusion, avoidance/numbing, and hyperarousal.

As used herein, the term “re-experiencing/intrusion” refers to at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders. In certain embodiments, the physiological reactivity manifests in at least one of abnormal respiration, abnormal cardiac rate of rhythm, abnormal blood pressure, abnormal function of a special sense, and abnormal function of sensory organ.

As used herein, the term “avoidance/numbing” refers to at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, and sense of a foreshortened future. Restricted range of effect characterized by diminished or restricted range or intensity of feelings or display of feelings can occur. A sense of a foreshortened future can manifest in thinking that one will not have a career, marriage, children, or a normal life span. Avoidance/numbing can also manifest in social anxiety and anxiety with unfamiliar surroundings.

As used herein, the term “hyperarousal” refers to at least one of difficulty falling or staying asleep, irritability or outbursts of anger, difficulty concentrating, hypervigilance, and exaggerated startle response. Potentially threatening stimuli can cause hyperarousal or anxiety.

As used herein, the term “significantly” refers to a set of observations or occurrences that are too closely correlated to be attributed to chance. For example, in certain embodiments, “significantly changes”, “significantly reduces”, and “significantly increases” refers to alterations or effects that are not likely to be attributed to chance. In certain embodiments, statistical methods can be used to determine whether an observation can be referred to as “significantly” changed, reduced, increased, or altered. In certain embodiments, a “score” that assesses post-traumatic stress disorder can be significantly changed, for example, by treatment for post-traumatic stress disorder.

Patients diagnosed with post-traumatic stress disorder may feel “on guard”, uneasy, and intensely anxious. Depression, anxiety, panic attacks, and bipolar disorder are often associated with post-traumatic stress disorder. Alcohol and drug abuse are also common. In certain embodiments, disorders cormorbid with post-traumatic stress disorder can include for example but without limitation depression, alcohol abuse, and drug abuse.

As used herein, the term “Clinician-Administered PTSD Scale (CAPS)” refers to a measure for diagnosing and assessing post-traumatic stress syndrome. The CAPS is a 30-item structured interview that corresponds to the DSM-IV criteria for PTSD. Different versions of this measure have been developed.

As used herein, the term “Clinician-Administered PTSD Scale-Part 1 (CAPS-1)” is a version of CAPS that assesses current and lifetime PTSD and is also known as CAPS-DX (for diagnosis).

As used herein, the term “Clinician-Administered PTSD Scale-Part 2 (CAPS-2)” refers to a version of CAPS used to assess one week symptom status in patients with post-traumatic stress disorder and also refers to a CAPS-SX (for symptom),

As used herein, the term “Clinician-Administered PTSD Scale for children and adolescents (CAPS-CA)” refers to a version of CAPS developed for children and adolescents.

As used herein, the term “Impact of Event Scale (IES)” refers to a scale developed by Mardi Horowitz, Nancy Wilner, and William Alvarez to measure subjective stress related to a specific event. It is a self-reported assessment and can be used to make measurements over time to monitor a patient's status.

As used herein, the term “Impact of Event Scale-Revised (IES-R)” refers to the revision of the IES developed by Daniel S. Weiss and Charles Marmar to assess the hyperarousal symptom cluster of PTSD.

As used herein, the term “Clinical Global Impression Scale (CGI)” refers to a scale for making psychiatric assessments. Patients are interviewed and the CGI is used to measure the severity of illness (CGI-S), global improvement (CGI-I), and efficacy index.

As used herein, the term “Clinical Global Impression Severity of Illness (CGI-S)” refers to an assessment of the patient's current symptoms. Generally, it is rated on a seven-point scale, ranging from a score of 1 (normal) to 7 (extremely ill). The severity of the patient's illness is compared to the severity of other patients' illness. For example, the CGI-S score can be used to measure a patient's condition after treatment with 4-hydroxy-4-methyl-piperidine-1-carboxylic acid (4-methoxy-7-morpholin-4-yl-benzothiazol-2-yl)-amide, and the scores before and after treatment may be compared.

As used herein, the term “Clinical Global Impression Improvement (CGI-I)” refers to a comparison of a patient's current condition to his baseline condition. Generally, it is rated on a seven-point scale ranging from 1 (very much improved) to 7 (very much worse). The CGI-I score can be used to measure, for example, improvement of post-traumatic stress disorder in response to 4-hydroxy-4-methyl-piperidine-1-carboxylic acid (4-methoxy-7-morpholin-4-yl-benzothiazol-2-yl)-amide treatment.

As used herein, the term “efficacy index” refers to a score taken on CGI and compares the patient's baseline condition with a ratio of current therapeutic benefit to severity of side effects. Generally, it is rated on a four-point scale ranging from 1 (none) to 4 (outweighs therapeutic effect). In assessing post-traumatic stress disorder, the efficacy index could, for example, assess the risk-benefit of treating with a therapy such as 4-hydroxy-4-methyl-piperidine-1-carboxylic acid (4-methoxy-7-morpholin-4-yl-benzothiazol-2-yl)-amide.

As used herein, the term “Duke Global Rating for PTSD scale (DGRP)” refers to a scale that measures severity and improvement for each of the three PTSD symptom clusters: re-experiencing/intrusion, avoidance/numbing, and hyperarousal as well as overall PTSD severity.

As used herein, the term “Duke Global Rating for PTSD scale-Improvement (DGRP-I)” refers to a scale used to distinguish responders (DGRP-I of 1 (very much improved) and 2 (much improved)) from nonresponders (DGRP-I>2) in response to a treatment, for example, 4-hydroxy-4-methyl-piperidine-1-carboxylic acid (4-methoxy-7-morpholin-4-yl-benzothiazol-2-yl)-amide, for post-traumatic stress disorder.

As used herein, the term “Hamilton Anxiety Scale (HAM-A)” refers to a scale developed by Max Hamilton in 1959 to diagnose and quantify symptoms of anxiety and post-traumatic stress disorder. It consists of 14 items, each defined by a series of symptoms. No standardized probe questions to elicit information from patients or behaviorally specific guidelines were developed for determining item scoring. Each item is rated on a 5-point scale, ranging from 0 (not present) to 4 (severe). Items include assessing anxious mood, fears, intellectual effects, somatic complaints, e.g. on musculature, cardiovascular symptoms, tension, insomnia, depressed mood, somatic sensory complaints, respiratory symptoms, gastrointestinal symptoms, autonomic symptoms, genitourinary symptoms, and behavior at the time of assessment. For example, a reduction in the HAM-A score would indicate improvement in a disorder such as post-traumatic stress disorder.

As used herein, the terms “Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI)” refer to additional scales that diagnose, assess, measure a sign, symptom, symptom cluster of post-traumatic stress disorder, anxiety, or depression.

As used herein, the term “score” refers to a score of at least one item or parameter measured on a scale that measures at least one sign, symptom, or symptom cluster of psychiatric symptoms, anxiety, or post-traumatic stress disorder. In certain embodiments, a score measures the frequency, intensity, or severity of a sign, symptom, symptom cluster, associated symptom, or impact on daily life of post-traumatic stress disorder.

As used herein, the term “endpoint score” refers to a score on an instrument that assesses post-traumatic stress disorder taken during or after treatment.

As used herein, the term “baseline score” refers to a score on an instrument that assesses post-traumatic stress disorder prior to initiation of a treatment.

As used herein, the term “overall score” refers to a sum of the scores on an instrument that assesses post-traumatic stress disorder. In certain embodiments, an overall score is the sum of a score of at least one of symptoms, symptom clusters, associated symptoms, impact on daily life, efficacy, and improvement.

As used herein, the term “relapse” refers to reoccurrence or worsening of at least one symptom of a disease or disorder in a patient.

As used herein the phrase “therapeutically effective amount” refers to the amount sufficient to provide a therapeutic outcome regarding at least one sign, symptom, or associated symptom of a disease, disorder, or condition. For example, in certain embodiments the disease, disorder, or condition is PTSD.

As used herein, the phrase “improving resilience” refers to increasing the ability of a patient to experience a traumatic event without suffering post-traumatic stress disorder or with less post-event symptomotology or disruption of normal activities of daily living. In certain embodiments, improving resilience can reduce the symptoms of post-traumatic stress disorder.

As used herein, the term “coadministering” refers to a dosage regimen for a first agent that overlaps with the dosage regimen of a second agent, or to simultaneous administration of the first agent and the second agent. A dosage regimen is characterized by dosage amount, frequency, and duration. Two dosage regimens overlap if between initiation of a first and initiation of a second administration of a first agent, the second agent is administered.

As used herein, the term “agent” refers to a substance including, but not limited to a chemical compound, such as a small molecule or a complex organic compound, a protein, such as an antibody or antibody fragment or a protein comprising an antibody fragment, or a genetic construct which acts at the DNA or mRNA level in an organism.

As used herein, the term “A_{2A} receptor activity” refers to at least one activity triggered by A_{2A} receptor. For example but not limited to, the activity may be adenylate cyclase activity, increase in cAMP levels, and calcium flux.

As used herein, the term “modulates” refers to changing or altering an activity, function, or feature. For example, an agent may modulate levels of a factor by elevating or reducing the levels of the factor.

As used herein, the term “dopaminergic signaling” refers to signal transduction triggered by dopamine and its effects on neuronal activities. There are five known dopaminergic receptors, 2 D1-like receptors (D1 and D5) and 3 D2-like receptors (D2, D3, and D4). Binding of dopamine to D1-like receptors stimulates adenylyl cyclase and binding to D2-like receptors inhibits adenylyl cyclase. Dopaminergic signaling causes changes in neuronal activities including but not limited to behavior, cognition, motor activity, motivation and reward, sleep, mood, attention, and learning.

Methods of treating a patient diagnosed with post-traumatic stress disorder are provided herein. The methods include administering to the patient a therapeutically effective amount of 4-hydroxy-4-methyl-piperidine-1-carboxylic acid (4-methoxy-7-morpholin-4-yl-benzothiazol-2-yl)-amide.

In certain embodiments the method further includes coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the at least one other agent is a SSRI selected from paroxetine, sertraline, citalopram, escitalopram, and fluoxetine.

In certain embodiments the at least one other agent is a SNRI selected from duloxetine, mirtazapine, and venlafaxine.

In certain embodiments the at least one other agent is a NRI selected from bupropion and atomoxetine.

In certain embodiments the at least one other agent is a dopamine β-hydroxylase inhibitor selected from nepicastat and disulfiram.

In certain embodiments the at least one other agent is the adenosine A2A receptor antagonist istradefylline.

In certain embodiments the at least one other agent is a sodium channel blocker selected from lamotrigine, carbamazepine, oxcarbazepine, and valproate.

In certain embodiments the at least one other agent is a calcium channel blocker selected from lamotrigine and carbamazepine.

In certain embodiments the at least one other agent is the central and peripheral alpha adrenergic receptor antagonist prazosin.

In certain embodiments the at least one other agent is the central alpha adrenergic agonist clonidine.

In certain embodiments the at least one other agent is the central or peripheral beta adrenergic receptor antagonist propranolol.

In certain embodiments the least one other agent is an atypical antidepressant/antipsychotic selected from olanzapine, risperidone, and quetiapene.

In certain embodiments the least one other agent is a tricyclic selected from amitriptyline, amoxapine, desipramine, doxepin, imipramine, nortriptyline, protriptyline, and trimipramine.

In certain embodiments the least one other agent is an anticonvulsant selected from lamotrigine, carbamazepine, oxcarbazepine, valproate, topiramate, and levetiracetam.

In certain embodiments the least one other agent is the glutamate antagonist topiramate.

In certain embodiments the least one other agent is a GABA agonist selected from valproate and topiramate.

In certain embodiments the least one other agent is the partial D2 agonist aripiprazole.

In certain embodiments the 4-hydroxy-4-methyl-piperidine-1-carboxylic acid (4-methoxy-7-morpholin-4-yl-benzothiazol-2-yl)-amide reduces at least one A_{2A} receptor activity in the patient.

In certain embodiments the 4-hydroxy-4-methyl-piperidine-1-carboxylic acid (4-methoxy-7-morpholin-4-yl-benzothiazol-2-yl)-amide modulates dopaminergic signaling in the patient.

In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one sign of the post-traumatic stress disorder in the patient. In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one symptom of the post-traumatic stress disorder in the patient. In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one symptom cluster of the post-traumatic stress disorder in the patient, wherein the symptom cluster is selected from re-experiencing/intrusion, avoidance/numbing, and hyperarousal.

In certain embodiments the re-experiencing/intrusion includes at least one of recurrent and intrusive trauma recollections, recurrent and distressing dreams of the traumatic event, acting or feeling as if the traumatic event were recurring, distress when exposed to trauma reminders, and physiological reactivity when exposed to trauma reminders.

In certain embodiments the physiological reactivity includes at least one of abnormal respiration, abnormal cardiac rate of rhythm, abnormal blood pressure, abnormal function of at least one special sense, and abnormal function of at least one sensory organ. In certain embodiments the at least one special sense is selected from sight, hearing, touch, smell, taste, and sense. In certain embodiments the at least one sensory organ is selected from eye, ear, skin, nose, tongue, and pharynx.

In certain embodiments the avoidance/numbing comprises at least one of efforts to avoid thoughts or feelings associated with the trauma, efforts to avoid activities or situations, inability to recall trauma or trauma aspects, markedly diminished interest in significant activities, feelings of detachment or estrangement from others, restricted range of affect, sense of a foreshortened future, social anxiety, and anxiety associated with unfamiliar surroundings.

In certain embodiments the hyperarousal comprises at least one of difficulty falling or staying asleep, irritability or outbursts of anger, difficulty concentrating, hypervigilance, exaggerated startle response, and anxiety from potentially threatening stimuli. In certain embodiments the 4-hydroxy-4-methyl-piperidine-1-carboxylic acid (4-methoxy-7-morpholin-4-yl-benzothiazol-2-yl)-amide does not reduce the physical ability of the patient to respond appropriately and promptly to the potentially threatening stimuli.

In certain embodiments the patient is a child or an adolescent. In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one sign or symptom of the post-traumatic stress disorder in the patient, wherein the sign or symptom is selected from disorganized or agitated behavior, repetitive play that expresses aspects of the trauma, frightening dreams which lack recognizable content, and trauma-specific reenactment.

In certain embodiments the 4-hydroxy-4-methyl-piperidine-1-carboxylic acid (4-methoxy-7-morpholin-4-yl-benzothiazol-2-yl)-amide reduces the incidence of at least one disorder comorbid with post-traumatic stress disorder selected from drug abuse, alcohol abuse, and depression in the patient.

In certain embodiments the 4-hydroxy-4-methyl-piperidine-1-carboxylic acid (4-methoxy-7-morpholin-4-yl-benzothiazol-2-yl)-amide is administered to the patient once or twice a day.

In certain embodiments the 4-hydroxy-4-methyl-piperidine-1-carboxylic acid (4-methoxy-7-morpholin-4-yl-benzothiazol-2-yl)-amide does not cause at least one of drowsiness, lassitude, or alteration of mental and physical capabilities.

In certain embodiments the 4-hydroxy-4-methyl-piperidine-1-carboxylic acid (4-methoxy-7-morpholin-4-yl-benzothiazol-2-yl)-amide is administered to the patient before or immediately after a traumatic event.

In certain embodiments the at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI). In certain embodiments the 4-hydroxy-4-methyl-piperidine-1-carboxylic acid (4-methoxy-7-morpholin-4-yl-benzothiazol-2-yl)-amide significantly changes a score on at least one of CAPS, CAPS-2, CAPS-CA, IES, IES-R, CGI, CGI-S, CGI-I, DGRP, DGRP-I, HAM-A, SI-PTSD, PTSD-I, PSS-I, MADRS, BDI, HAM-D, RHRSD, MDI, GDS-30, and CDI. In certain embodiments the 4-hydroxy-4-methyl-piperidine-1-carboxylic acid (4-methoxy-7-morpholin-4-yl-benzothiazol-2-yl)-amide significantly reduces an endpoint score compared to a baseline score on at least one of CAPS, CAPS-2, IES, IES-R, and HAMA. In certain embodiments the 4-hydroxy-4-methyl-piperidine-1-carboxylic acid (4-methoxy-7-morpholin-4-yl-benzothiazol-2-yl)-amide significantly increases the proportion of responders on the CGI-I having CGI-I scores of at least one of 1 (very much improved) and 2 (much improved). In certain embodiments the 4-hydroxy-4-methyl-piperidine-1-carboxylic acid (4-methoxy-7-morpholin-4-yl-benzothiazol-2-yl)-amide increases the proportion of responders on the DGRP-I having a DGRP-I scores of at least one of 1 (very much improved) and 2 (much improved). In certain embodiments an overall score of at least 65 on at least one of the CAPS and the CAP-2 is indicative of post-traumatic stress disorder. In certain embodiments an overall score of at least 18 on HAM-A is indicative of anxiety disorder. In certain embodiments a score of at least 3 on at least one of the CGI-I and the DGRP-I is indicative of post-traumatic stress disorder.

Also provided are methods of treating post-traumatic stress disorder in a patient. The methods include diagnosing the patient with post-traumatic stress disorder; administering to the patient a therapeutically effective amount of 4-hydroxy-4-methyl-piperidine-1-carboxylic acid (4-methoxy-7-morpholin-4-yl-benzothiazol-2-yl)-amide; assessing at least one of sign, symptom, and symptom cluster of post-traumatic stress disorder; and determining that the post-traumatic stress disorder is improved if the 4-hydroxy-4-methyl-piperidine-1-carboxylic acid (4-methoxy-7-morpholin-4-yl-benzothiazol-2-yl)-amide reduces at least one of sign, symptom, and symptom cluster of post-traumatic stress disorder.

In certain embodiments the methods further include coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one sign of the post-traumatic stress disorder in the patient.

In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one symptom of the post-traumatic stress disorder in the patient.

In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one symptom cluster of the post-traumatic stress disorder in the patient, wherein the symptom cluster is selected from re-experiencing/intrusion, avoidance/numbing, and hyperarousal. In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

Also provided are methods of improving resilience in a patient. The methods include administering a therapeutically effective amount of 4-hydroxy-4-methyl-piperidine-1-carboxylic acid (4-methoxy-7-morpholin-4-yl-benzothiazol-2-yl)-amide. In certain embodiments the methods further include coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one sign of the post-traumatic stress disorder in the patient.

In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one symptom of the post-traumatic stress disorder in the patient.

In certain embodiments the 4-hydroxy-4-methyl-piperidine-1-carboxylic acid (4-methoxy-7-morpholin-4-yl-benzothiazol-2-yl)-amide reduces at least one of the frequency and intensity of at least one symptom cluster of the post-traumatic stress disorder in the patient, wherein the symptom cluster is selected from re-experiencing/intrusion, avoidance/numbing, and hyperarousal. In certain embodiments at least one sign, symptom, or symptom cluster of post-traumatic stress syndrome is diagnosed or assessed with at least one of Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI).

Also provided are methods of diagnosing post-traumatic stress disorder in a patient. The methods include administering to the patient a therapeutically effective amount of 4-hydroxy-4-methyl-piperidine-1-carboxylic acid (4-methoxy-7-morpholin-4-yl-benzothiazol-2-yl)-amide and assessing at least one of sign, symptom, or symptom cluster of post-traumatic stress disorder; and diagnosing post-traumatic stress disorder in the patient if the 4-hydroxy-4-methyl-piperidine-1-carboxylic acid (4-methoxy-7-morpholin-4-yl-benzothiazol-2-yl)-amide reduces at least one of sign, symptom, and symptom cluster of post-traumatic stress disorder. In certain embodiments the patient is a child, adolescent, or adult.

Various scales can assess post-traumatic stress disorder (PTSD) and the effect of rufinamide and other therapies on the treatment and prevention of the disorder. These are, for example and without limitation, Clinician-Administered PTSD Scale (CAPS), Clinician-Administered PTSD Scale Part 2 (CAPS-2), Clinician-Administered PTSD Scale for Children and Adolescents (CAPS-CA), Impact of Event Scale (IES), Impact of Event Scale-Revised (IES-R), Clinical Global Impression Scale (CGI), Clinical Global Impression Severity of Illness (CGI-S), Clinical Global Impression Improvement (CGI-I), Duke Global Rating for PTSD scale (DGRP), Duke Global Rating for PTSD scale Improvement (DGRP-I), Hamilton Anxiety Scale (HAM-A), Structured Interview for PTSD (SI-PTSD), PTSD Interview (PTSD-I), PTSD Symptom Scale (PSS-I), Mini International Neuropsychiatric Interview (MINI), Montgomery-Åsberg Depression Rating Scale (MADRS), Beck Depression Inventory (BDI), Hamilton Depression Scale (HAM-D), Revised Hamilton Rating Scale for Depression (RHRSD), Major Depressive Inventory (MDI), Geriatric Depression Scale (GDS-30), and Children's Depression Index (CDI). These measures generally are assessed by interviews or questionnaires. In certain embodiments, not all the parts of a scale are administered. In certain embodiments, the scales are used for diagnosing and assessing signs, symptoms, associated symptoms, or impact on daily life of PTSD. In certain embodiments, one or more scales are used to diagnose, assess, or confirm post-traumatic stress disorder in a patient. In certain embodiments, scales will measure signs, symptoms, symptom clusters by scoring at least one of the frequency and intensity of the signs, symptoms, or symptom clusters.

Examples of scales for post-traumatic stress disorder assessment are versions of CAPS, including CAPS, CAPS-1, and CAPS-2, which score 17 core PTSD symptoms with these items:

1. Recurrent and intrusive trauma recollections

2. Distress when exposed to trauma reminders

3. Acting or feeling as if event were recurring

4. Recurrent and distressing dreams of event

5. Efforts to avoid thoughts or feelings

6. Efforts to avoid activities or situations

7. Inability to recall trauma or trauma aspects

8. Markedly diminished interest in significant activities

9. Feelings of detachment or estrangement from others

10. Restricted range of affect

11. Sense of a foreshortened future

12. Difficulty falling or staying asleep

13. Irritability or outbursts of anger

14. Difficulty concentrating

15. Hypervigilance

16. Exaggerated startle response

17. Physiologic reactivity

Questions also target the impact of symptoms on social and occupational functioning or daily life, improvement in symptoms since a previous CAPS administration, overall response validity, overall PTSD severity, and frequency and intensity of associated symptoms. These items are:

18. Impact on Social Functioning

19. Impact on Occupational Functioning

20. Global Improvement (since earlier measurement occasion)

21. Rating Validity

22. Global Improvement

23. Guilt over acts committed or omitted

24. Survivor Guilt

25. Homicidality

26. Disillusionment with authority

27. Feelings of hopelessness

28. Memory Impairment

29. Sadness and depression

30. Feelings of being overwhelmed

To assess the frequency of symptoms, interviewers follow standard questions, clarifying or rephrasing as needed. Standard questions, by way of example and without limitation, are: Have you ever had unwanted memories of the traumatic event? What were they like? What did you remember? If the question requires rephrasing, the interviewer can ask a question such as: Did they ever occur while you were awake or only in dreams? or How often have you had these memories in the past month (week)? A score of 0 indicates a frequency of never, 1 indicates once or twice, 2 indicates once or twice a week, 3 indicates several times a week, and 4 indicates daily of almost every day.

To assess the intensity of symptoms, an interviewer may ask standard questions such as by way of example and without limitation: How much distress or discomfort did these memories cause you? Were you able to put them out of your mind and think about something else? How hard did you have to try? How much did they interfere with your life? A score of 0 indicates none, 1 indicates mild, minimal distress or disruption of activities, 2 indicates moderate, distress clearly present but still manageable, some disruption of activities, 3 indicates severe, considerable distress, difficulty dismissing memories, marked disruption of activities, and 4 indicates extreme, incapacitating distress, cannot dismiss memories, unable to continue activities.

In certain embodiments the scoring rule used counts a symptom as present if it has a frequency of 1 or more and an intensity of 2 or more. In other embodiments severity scores are calculated by summing the frequency and intensity ratings for each symptom. In certain embodiments, a total or overall score of all items on a version of CAPS is calculated. In certain embodiments, a total score for each symptom cluster is calculated. In certain embodiments, a total score for core symptoms of PTSD is calculated. In certain embodiments, an endpoint score is compared to a baseline score to determine the change in severity of post-traumatic stress disorder. In certain embodiments, a significant reduction of an endpoint score compared to a baseline score is considered improvement of PTSD. In certain embodiments, an overall score on CAPS, CAPS-1, CAPS-2, or CAPS-CA greater than 65 is indicative of PTSD.

Another example is the IES which assesses 15 items: 7 items measure intrusive symptoms and 8 items measure avoidance symptoms. The self assessed items ask how frequently each of the following comments are true: I thought about it when I didn't mean to, I avoided letting myself get upset when I thought about it or was reminded of it, I tried to remove it from memory, I had trouble falling asleep or staying asleep because of pictures or thoughts about it that came into my mind, I had waves of strong feelings about it, I had dreams about it, I stayed away from reminders of it, It felt as it hadn't happened or wasn't real, I tried not to talk about it, Pictures about it popped into my mind, Others things kept making me think about it, I was aware that I still had a lot of feelings about it, but I didn't deal with them, I tried not to think about it, Any reminder brought back feelings about it, and My feelings were kind of numb. The items are generally rated on a four point scale: 0 (not at all), 1 (rarely), 3 (sometimes), and 5 (often). The total of the scores provide an overall assessment of the severity of the symptoms or overall subjective stress. It has been suggested that a score from 0 to 8 is in the subclinical range, 9-25 is in the mild range, 26-43 is in the moderate range, and greater than 44 is in the severe range of stress.

In certain embodiments, a total or overall score of all items on IES is calculated. In certain embodiments, a total score for each symptom cluster is calculated. In certain embodiments, an endpoint score is compared to a baseline score to determine the change in severity of PTSD. In certain embodiments, a reduction of an endpoint score by 30% compared to a baseline score is considered improvement of PTSD. The IES-R, a revision of the IES, changed the IES by splitting the original IES item, I had trouble falling asleep or staying asleep into two items: I had trouble falling asleep and I had trouble staying asleep and by adding six items to the IES items. These additional items are: I felt irritable and angry, I was jumpy and easily startled, I found myself acting or feeling as though I was back at that time, I had trouble concentrating, Reminders of it caused me to have physical reactions, such as sweating, trouble breathing, nausea, or a pounding heart, and I felt watchful or on guard. The scoring system also changed to 0 (not at all), 1 (a little bit), 2 (moderately), 3 (quite a bit), and 4 (extremely).

In certain embodiments, a total or overall score of all items on IES-R is calculated. In certain embodiments, a total score for each symptom cluster is calculated. In certain embodiments, an endpoint score is compared to a baseline score to determine the change in severity of post-traumatic stress disorder. In certain embodiments, a significant reduction of an endpoint score compared to a baseline score on the IES-R is considered improvement of post-traumatic stress disorder.

In the DGRP-I scale, the effectiveness of 4-hydroxy-4-methyl-piperidine-1-carboxylic acid (4-methoxy-7-morpholin-4-yl-benzothiazol-2-yl)-amide in treating post-traumatic stress disorder can be assessed by measuring the increase in the proportion of responders on the DGRP-I having a DGRP-I of 1 (very much improved) or 2 (much improved). In certain embodiments, a score of at least 3 on the DGRP-I is indicative of post-traumatic stress

In the CGI, the effectiveness of 4-hydroxy-4-methyl-piperidine-1-carboxylic acid (4-methoxy-7-morpholin-4-yl-benzothiazol-2-yl)-amide to treat post-traumatic stress disorder can be assessed by the CGI-S, CGI-I, and efficacy index. For example, in certain embodiments, an increase in the proportion of responders on the CGI-I having a CGI-I of 1 (very much improved) or 2 (much improved) after treatment indicates that the treatment is effective. In certain embodiments, a score of at least 3 on the CGI-I is indicative of post-traumatic stress disorder. In certain embodiments, the efficacy index on the CGI can measure the efficacy of 4-hydroxy-4-methyl-piperidine-1-carboxylic acid (4-methoxy-7-morpholin-4-yl-benzothiazol-2-yl)-amide for treatment of post-traumatic stress disorder.

In HAMA-A, to assess anxiety or post-traumatic stress disorder, generally a total or overall score of all items on HAM-A is calculated. In certain embodiments, an endpoint score is compared to a baseline score on HAM-A to determine the change in severity of anxiety and post-traumatic stress disorder. In certain embodiments, a significant reduction of an endpoint score compared to a baseline score on HAM-A is considered improvement of anxiety and post-traumatic stress disorder. In certain embodiments, an overall score on HAM-A of at least 18 is indicative of anxiety and post-traumatic stress disorder.

Pharmaceutically acceptable forms of 4-hydroxy-4-methyl-piperidine-1-carboxylic acid (4-methoxy-7-morpholin-4-yl-benzothiazol-2-yl)-amide include acids, bases, enol ethers, and esters, esters, hydrates, solvates, and prodrug forms. The derivative is selected such that its pharmokinetic properties are superior with respect to at least one characteristic to the corresponding neutral agent. The 4-hydroxy-4-methyl-piperidine-1-carboxylic acid (4-methoxy-7-morpholin-4-yl-benzothiazol-2-yl)-amide may be derivatized prior to formulation.

In general, 4-hydroxy-4-methyl-piperidine-1-carboxylic acid (4-methoxy-7-morpholin-4-yl-benzothiazol-2-yl)-amide or a pharmaceutically acceptable derivative will be administered in therapeutically effective amounts, either singly or in combination with another therapeutic agent. The pharmaceutical compositions will be useful, for example, for the treatment of post-traumatic stress disorder.

A therapeutically effective amount of 4-hydroxy-4-methyl-piperidine-1-carboxylic acid (4-methoxy-7-morpholin-4-yl-benzothiazol-2-yl)-amide or a pharmaceutically acceptable form may vary widely depending on the severity of the post-traumatic stress disorder, the age and relative health of the subject, the potency of the compound used and other factors. In certain embodiments a therapeutically effective amount is from about 0.1 milligram per kg (mg/kg) body weight per day to about 50 mg/kg body weight per day. In other embodiments the amount is about 1.0 to about 10 mg/kg/day. Therefore, in certain embodiments a therapeutically effective amount for a 70 kg human is from about 7.0 to about 3500 mg/day, while in other embodiments it is about 70 to about 700 mg/day.

One of ordinary skill in the art of treating such diseases will be able to ascertain a therapeutically effective amount of 4-hydroxy-4-methyl-piperidine-1-carboxylic acid (4-methoxy-7-morpholin-4-yl-benzothiazol-2-yl)-amide for post-traumatic stress disorder without undue experimentation and in reliance upon personal knowledge and the disclosure of this application. In general, by way of example and without limitation, 4-hydroxy-4-methyl-piperidine-1-carboxylic acid (4-methoxy-7-morpholin-4-yl-benzothiazol-2-yl)-amide will be administered as pharmaceutical compositions by one of the following routes: oral, systemic (e.g., transdermal, intranasal or by suppository) or parenteral (e.g., intramuscular, intravenous or subcutaneous). Compositions can, by way of example and without limitation, take the form of tablets, pills, capsules, semisolids, powders, sustained release formulations, solutions, suspensions, elixirs, aerosols, or any other appropriate composition and are comprised of, in general, 4-hydroxy-4-methyl-piperidine-1-carboxylic acid (4-methoxy-7-morpholin-4-yl-benzothiazol-2-yl)-amide in combination with at least one pharmaceutically acceptable excipient. Acceptable excipients are, by way of example and without limitation, non-toxic, aid administration, and do not adversely affect the therapeutic benefit of the compound. Such excipient may be, for example, any solid, liquid, semisolid or, in the case of an aerosol composition, gaseous excipient that is generally available to one of skill in the art.

Solid pharmaceutical excipients include by way of example and without limitation starch, cellulose, talc, glucose, lactose, sucrose, gelatin, malt, rice, flour, chalk, silica gel, magnesium stearate, sodium stearate, glycerol monostearate, sodium chloride, dried skim milk, and the like. Liquid and semisolid excipients may be selected from for example and without limitation water, ethanol, glycerol, propylene glycol and various oils, including those of petroleum, animal, vegetable or synthetic origin (e.g., peanut oil, soybean oil, mineral oil, sesame oil, etc.). Preferred liquid carriers, particularly for injectable solutions, include by way of example and without limitation water, saline, aqueous dextrose and glycols. Compressed gases may be used to disperse the compound in aerosol form. Inert gases suitable for this purpose are by way of example and without limitation nitrogen, carbon dioxide, nitrous oxide, etc.

The pharmaceutical preparations can by way of example and without limitation, moreover, contain preservatives, solubilizers, stabilizers, wetting agents, emulsifiers, sweeteners, colorants, flavorants, salts for varying the osmotic pressure, buffers, masking agents or antioxidants. In certain embodiments, they can contain still other therapeutically valuable substances. Other suitable pharmaceutical carriers and their formulations are described in A. R. Alfonso Remington's Pharmaceutical Sciences 1985, 17th ed. Easton, Pa.: Mack Publishing Company.

The amount of 4-hydroxy-4-methyl-piperidine-1-carboxylic acid (4-methoxy-7-morpholin-4-yl-benzothiazol-2-yl)-amide in the composition may vary widely depending for example, upon the type of formulation, size of a unit dosage, kind of excipients and other factors known to those of skill in the art of pharmaceutical sciences. In general, the final composition will comprise from 10% w to 90% w of the compound, preferably 25% w to 75% w, with the remainder being the excipient or excipients. Preferably the pharmaceutical composition is administered in a single unit dosage form for continuous treatment or in a single unit dosage form ad libitum when relief of symptoms is specifically required.

The 4-hydroxy-4-methyl-piperidine-1-carboxylic acid (4-methoxy-7-morpholin-4-yl-benzothiazol-2-yl)-amide or a pharmaceutically acceptable form thereof is administered simultaneously with, prior to, or after administration of one or more of the above agents.

The invention is further illustrated by the following non-limiting examples.

### EXAMPLES

### Example 1

A clinical study is performed to demonstrate the efficacy and tolerability of 4-hydroxy-4-methyl-piperidine-1-carboxylic acid (4-methoxy-7-morpholin-4-yl-benzothiazol-2-yl)-amide in the treatment of post-traumatic stress disorder (PTSD).

The research design includes an 8-week randomized, double-blind, placebo-controlled treatment trial of 4-hydroxy-4-methyl-piperidine-1-carboxylic acid (4-methoxy-7-morpholin-4-yl-benzothiazol-2-yl)-amide for the treatment of PTSD.

After signing an informed consent and meeting inclusion/exclusion criteria, patients are randomized to receive either 4-hydroxy-4-methyl-piperidine-1-carboxylic acid (4-methoxy-7-morpholin-4-yl-benzothiazol-2-yl)-amide or placebo for the 8-week duration. During the study a pharmacist maintains the randomization log and verify the order for the placebo or 4-hydroxy-4-methyl-piperidine-1-carboxylic acid (4-methoxy-7-morpholin-4-yl-benzothiazol-2-yl)-amide in look-a-like tablets. Patients' symptoms, side effects and compliance is assessed bi-weekly.

Based on symptomotology and occurrence of side effects, the investigator may increase the medication in 20-40 mg increments, as tolerated, until a maximum therapeutic benefit is achieved. The dosing is once per day unless twice per day is better tolerated. Compliance is assessed by pill count at week 4 and week 8.

Efficacy is measured by at least one of the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)

The subject inclusion criteria are:
Diagnosis of PTSD that is confirmed by Mini International Neuropsychiatric Interview (MINI) and CAPS
Age 13 or older
No substance abuse or dependence for the previous 4 weeks (except for nicotine and caffeine)
Free of psychotropic medication for 2 weeks (except 4 weeks for fluoxetine)
Clinically normal physical and laboratory examination (Liver function tests (LFTs) up to 2.5 times the normal limit is allowed.)
Women of childbearing potential must be using medically approved methods of birth control such as a condom, birth control pill, Depo-Provera, or diaphragm with spermicides
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to 4-hydroxy-4-methyl-piperidine-1-carboxylic acid (4-methoxy-7-morpholin-4-yl-benzothiazol-2-yl)-amide
Unstable general medical conditions
Score≧6 on Question #10 of MADRS regarding suicidal ideation
Women who are pregnant, planning to become pregnant or breastfeed during the study

Fulfillment of only one exit criterion is needed to exit the study. Exit criteria are:
Completion of the study
Severe and intolerable side effects to 4-hydroxy-4-methyl-piperidine-1-carboxylic acid (4-methoxy-7-morpholin-4-yl-benzothiazol-2-yl)-amide or placebo treatment
Acute development of suicidal ideation, homicidal ideation or psychotic symptoms
Worsening of symptoms as measured by a score of 7 (very much worse) on CGI-I
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 2

A clinical study is performed to demonstrate the efficacy and tolerability of 4-hydroxy-4-methyl-piperidine-1-carboxylic acid (4-methoxy-7-morpholin-4-yl-benzothiazol-2-yl)-amide in the prevention of PTSD.

The research design includes an open-ended randomized, double-blind, placebo-controlled treatment trial of 4-hydroxy-4-methyl-piperidine-1-carboxylic acid (4-methoxy-7-morpholin-4-yl-benzothiazol-2-yl)-amide for the prevention of PTSD. After signing an informed consent and meeting inclusion/exclusion criteria, patients are randomized to receive either 4-hydroxy-4-methyl-piperidine-1-carboxylic acid (4-methoxy-7-morpholin-4-yl-benzothiazol-2-yl)-amide versus placebo for the 8-week duration. During the study a pharmacist maintains the randomization log and verify the order for the placebo or 4-hydroxy-4-methyl-piperidine-1-carboxylic acid (4-methoxy-7-morpholin-4-yl-benzothiazol-2-yl)-amide in look-a-like tablets. Patients' symptoms, side effects and compliance are assessed bi-weekly.

Based on symptomotology and occurrence of side effects, the investigator can increase the medication in 20-40 mg increments, as tolerated, until a maximum therapeutic benefit is achieved. The dosing is once per day unless twice per day is better tolerated. Compliance is assessed by pill count at week 4 and week 8.

Efficacy is measured by at least one of the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)
Diagnostic and Statistical Manual IV (DSM-IV)

The subject inclusion criteria are:
Absence of PTSD, confirmed by MINI and CAPS
Age 13 or older
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The exclusion criteria are:
History of PTSD
Lifetime history of bipolar I, psychotic, or cognitive disorders
Actively suicidal, homicidal, or psychotic
History of sensitivity to 4-hydroxy-4-methyl-piperidine-1-carboxylic acid (4-methoxy-7-morpholin-4-yl-benzothiazol-2-yl)-amide
Unstable general medical conditions
Score≧6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to 4-hydroxy-4-methyl-piperidine-1-carboxylic acid (4-methoxy-7-morpholin-4-yl-benzothiazol-2-yl)-amide or placebo treatment
Acute development of suicidal ideation, homicidal ideation or psychotic symptoms
Appearance of signs or symptoms compatible with a diagnosis of PTSD.
Participant's explicit request to exit the study
The need for additional psychotropic drugs, other than the study drug or adjunctive medication as specified in the protocol, for the control of the subjects psychiatric symptoms.
The subject becomes pregnant during the course of the study.
Investigator's judgment that it is no longer in the best interest of the patient to continue in the study.

### Example 3

A clinical study is conducted to demonstrate the efficacy and tolerability of 4-hydroxy-4-methyl-piperidine-1-carboxylic acid (4-methoxy-7-morpholin-4-yl-benzothiazol-2-yl)-amide combination therapy in the treatment of PTSD.

The research design includes an 8-week randomized, double-blind, placebo-controlled treatment trial of 4-hydroxy-4-methyl-piperidine-1-carboxylic acid (4-methoxy-7-morpholin-4-yl-benzothiazol-2-yl)-amide for the treatment of PTSD. After signing an informed consent and meeting inclusion/exclusion criteria, patients are randomized to receive either 4-hydroxy-4-methyl-piperidine-1-carboxylic acid (4-methoxy-7-morpholin-4-yl-benzothiazol-2-yl)-amide or placebo for 8-week duration. Patients can also receive therapeutically effective doses of prazosin, valproate, carbamazepine, or topiramate in combination with 4-hydroxy-4-methyl-piperidine-1-carboxylic acid (4-methoxy-7-morpholin-4-yl-benzothiazol-2-yl)-amide or placebo.

During the study a pharmacist maintains the randomization log and verifies the order for the placebo or 4-hydroxy-4-methyl-piperidine-1-carboxylic acid (4-methoxy-7-morpholin-4-yl-benzothiazol-2-yl)-amide in look-a-like tablets. Patients' symptoms, side effects and compliance are assessed bi-weekly. Based on symptomotology and occurrence of side effects, the investigator increases the medication in 20-40 mg increments, as tolerated, until a maximum therapeutic benefit is achieved. The dosing is once per day unless twice per day is better tolerated. Compliance is assessed by pill count at week 4 and week 8.

Efficacy is measured by at least one of the following assessment scales:
Global Assessment of Functioning (GAF)
Clinician Administered PTSD Scale (CAPS)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)

The subject inclusion criteria are:
Diagnosis of PTSD, confirmed by MINI and CAPS
Age 13 or older
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to 4-hydroxy-4-methyl-piperidine-1-carboxylic acid (4-methoxy-7-morpholin-4-yl-benzothiazol-2-yl)-amide
Unstable general medical conditions
Score≧6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to 4-hydroxy-4-methyl-piperidine-1-carboxylic acid (4-methoxy-7-morpholin-4-yl-benzothiazol-2-yl)-amide or placebo treatment
Acute development of suicidal ideation, homicidal ideation or psychotic symptoms
Symptoms worsen as measured by a Score of 7 (very much worse) on CGI-I
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 4

A clinical study is performed to demonstrate the efficacy and tolerability of 4-hydroxy-4-methyl-piperidine-1-carboxylic acid (4-methoxy-7-morpholin-4-yl-benzothiazol-2-yl)-amide in the treatment of PTSD in children.

The research design includes an 8-week randomized, double-blind, placebo-controlled treatment trial of 4-hydroxy-4-methyl-piperidine-1-carboxylic acid (4-methoxy-7-morpholin-4-yl-benzothiazol-2-yl)-amide for the treatment of PTSD.

After signing an informed consent and meeting inclusion/exclusion criteria, the patients are randomized to receive either 4-hydroxy-4-methyl-piperidine-1-carboxylic acid (4-methoxy-7-morpholin-4-yl-benzothiazol-2-yl)-amide or placebo for an 8-week duration. During the study a pharmacist maintains the randomization log and verify the order for the placebo or 4-hydroxy-4-methyl-piperidine-1-carboxylic acid (4-methoxy-7-morpholin-4-yl-benzothiazol-2-yl)-amide in look-a-like tablets. Patients' symptoms, side effects and compliance are assessed bi-weekly.

Based on symptomotology and occurrence of side effects, the investigator can increase the medication in 20-40 mg increments, as tolerated, until a maximum therapeutic benefit is achieved. The dosing is once per day unless twice per day is better tolerated. Compliance is assessed by pill count at week 4 and week 8.

Patients are given supportive clinical management during the clinic visits. An investigator is available by telephone 24 hrs a day in case of emergency. Patients may be seen more often if needed.

Efficacy is measured by at least one of the following assessment scales:
Global Assessment of Functioning (GAF)
Clinician Administered PTSD Scale (CAPS)
Clinician Administered PTSD Scale (CAPS-CA)
Clinical Global Impression Severity of Illness (CGI-s)
Clinical Global Impression of Improvement (CGI-I)
Davidson Trauma Scale (DTS).
Hamilton Anxiety Scale (Ham-A)
Montgomery-Asberg Depression Rating Scale (MADRS)
Treatment Outcome PTSD rating scale (TOP-8)

The subject inclusion criteria are:
Diagnosis of PTSD, confirmed by MINI and CAPS
Age 12 or younger
No substance abuse/dependence for the previous 4 weeks (except for nicotine and caffeine)
Free of psychotropic medication for 2 weeks (except 4 weeks for fluoxetine)
Clinically normal physical and laboratory examination (LFTs up to 2.5 times the normal limit is allowed.)
Women of childbearing potential must be using medically approved methods of birth control (such as a condom, birth control pill, Depo-Provera, or diaphragm with spermicides)
Signed informed consent
Male or female, any race or ethic origin

The subject exclusion criteria are:
Lifetime history of bipolar I, psychotic, or cognitive disorders
Actively suicidal, homicidal, or psychotic
History of sensitivity to 4-hydroxy-4-methyl-piperidine-1-carboxylic acid (4-methoxy-7-morpholin-4-yl-benzothiazol-2-yl)-amide
Unstable general medical conditions
Score≧6 on Question #10 of MADRS regarding suicidal ideation
Women who are pregnant, planning to become pregnant or breastfeed during the study

Fulfillment of only one exit criterion is needed to exit the study. Exit Criteria are:
Completion of the study
Severe and intolerable side effects to 4-hydroxy-4-methyl-piperidine-1-carboxylic acid (4-methoxy-7-morpholin-4-yl-benzothiazol-2-yl)-amide or placebo treatment
Acute development of suicidal ideation, homicidal ideation or psychotic symptoms
Symptoms worsen as measured by a Score of 7 (very much worse) on CGI-I.
Participant's explicit request to exit the study
The need for additional psychotropic drugs, other than the study drug or adjunctive medication as specified in the protocol, for the control of the subjects psychiatric symptoms
The subject becomes pregnant during the course of the study
Investigator's judgment that it is no longer in the best interest of the patient to continue in the study

### Example 5

Studies were carried out in vitro and in vivo to investigate the specificity, selectivity and activity of 4-hydroxy-4-methyl-piperidine-1-carboxylic acid (4-methoxy-7-morpholin-4-yl-benzothiazol-2-yl)-amide. The in vitro studies examined binding and functional inactivation of A_{2A} receptors, and unspecific interaction with other binding sites. The in vivo studies investigated the potency of 4-hydroxy-4-methyl-piperidine-1-carboxylic acid (4-methoxy-7-morpholin-4-yl-benzothiazol-2-yl)-amide to antagonize A_{2A} receptor agonist-induced behavioral effects, and its efficacy in several animal models of depression, anxiety, and cognition.

Radioligand binding assays showed that 4-hydroxy-4-methyl-piperidine-1-carboxylic acid (4-methoxy-7-morpholin-4-yl-benzothiazol-2-yl)-amide has a high affinity for the human A_{2A} receptor (pKi 8.3) with approximately 230, 110 and 260-fold selectivity compared to hA₁, hA_{2B}, and hA₃, respectively. 4-hydroxy-4-methyl-piperidine-1-carboxylic acid (4-methoxy-7-morpholin-4-yl-benzothiazol-2-yl)-amide also has a high affinity for A_{2A} receptors in the rat (pKi 7.7), dog (pKi 7.9) and monkey (pKi 7.9).

Further radioligand binding studies assessed the selectivity of 4-hydroxy-4-methyl-piperidine-1-carboxylic acid (4-methoxy-7-morpholin-4-yl-benzothiazol-2-yl)-amide over more than 67 receptors, neurotransmitter transporters and ion channels. 4-hydroxy-4-methyl-piperidine-1-carboxylic acid (4-methoxy-7-morpholin-4-yl-benzothiazol-2-yl)-amide has a 1900-fold selectivity for the A_{2A} receptor over the targets tested (except for the adenosine transporter, where 4-hydroxy-4-methyl-piperidine-1-carboxylic acid (4-methoxy-7-morpholin-4-yl-benzothiazol-2-yl)-amide showed 55% displacement at 10 μM). Cellular biology assays on 16 enzyme targets showed that 4-hydroxy-4-methyl-piperidine-1-carboxylic acid (4-methoxy-7-morpholin-4-yl-benzothiazol-2-yl)-amide has a 1900-fold selectivity for the A_{2A} receptor over these targets, and only inhibition (88% at 10 μM) for the phosphodiesterase (IV) enzyme was detected.

A functional assay assessed the ability of 4-hydroxy-4-methyl-piperidine-1-carboxylic acid (4-methoxy-7-morpholin-4-yl-benzothiazol-2-yl)-amide to antagonize NECA (a non-specific adenosine receptor agonist) stimulated Ca²⁺ flux in hA_{2A}-Gα16-CHO cells. 4-hydroxy-4-methyl-piperidine-1-carboxylic acid (4-methoxy-7-morpholin-4-yl-benzothiazol-2-yl)-amide inhibited A_{2A}-mediated responses with a pIC₅₀ value of 8.83 (Hill slope 0.6). 4-hydroxy-4-methyl-piperidine-1-carboxylic acid (4-methoxy-7-morpholin-4-yl-benzothiazol-2-yl)-amide antagonized the NECA-stimulated Ca²⁺ flux in hA₁-Gα16-CHO cells with pIC₅₀ value of 5.22 (Hill slope 0.7). These data indicate that in this assay 4-hydroxy-4-methyl-piperidine-1-carboxylic acid (4-methoxy-7-morpholin-4-yl-benzothiazol-2-yl)-amide exhibits >4000-fold selectivity for the hA_{2A} receptor over hA₁.

APEC (2-[(2-aminoethylamino)carbonylethyl-phenyl-ethylamino]-5′-ethylcarboxamido-adenosine), an A_{2A} receptor agonist, reduces spontaneous motor activity in a dose-dependent manner. APEC-induced hypolocomotion is attenuated by selective A_{2A} receptor antagonists but not by selective A₁ receptor antagonists. The ability of 4-hydroxy-4-methyl-piperidine-1-carboxylic acid (4-methoxy-7-morpholin-4-yl-benzothiazol-2-yl)-amide to block APEC-induced hypolocomotion in rats was assessed to determine its in vivo potency and its efficacy as a selective A_{2A} receptor antagonist.

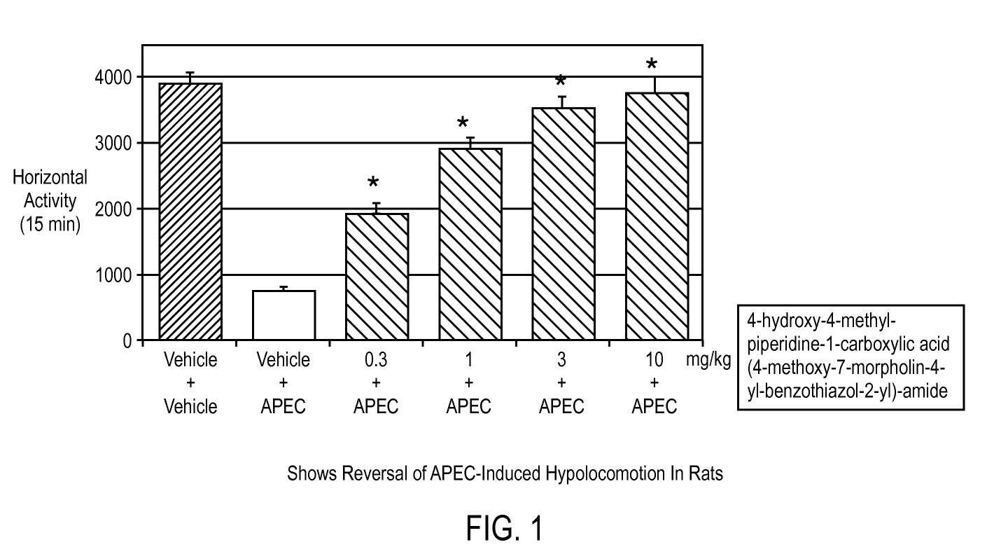

Administration of oral (po) 4-hydroxy-4-methyl-piperidine-1-carboxylic acid (4-methoxy-7-morpholin-4-yl-benzothiazol-2-yl)-amide to Wistar rats significantly reversed APEC-induced deficits in locomotor activity compared with controls, with ID₅₀ and ID₉₀ values of 0.5 and 3.4 mg/kg, respectively (FIG. 1). This test confirms the in vivo efficacy of 4-hydroxy-4-methyl-piperidine-1-carboxylic acid (4-methoxy-7-morpholin-4-yl-benzothiazol-2-yl)-amide po as well as its selectivity as an A_{2A} receptor antagonist.

Male Wistar rats were treated with 4-hydroxy-4-methyl-piperidine-1-carboxylic acid (4-methoxy-7-morpholin-4-yl-benzothiazol-2-yl)-amide administered po in doses ranging from 0.3 to 10 mg/kg, followed by a subcutaneous injection of 0.01 mg/kg of APEC. Control animals received vehicle only or vehicle and APEC. The animals were placed in plexiglass test cages with arrays of photocells linked to a computer, and motor activity was recorded for 15 min. Data are mean±SEM based on 8 animals per group. Administration of 4-hydroxy-4-methyl-piperidine-1-carboxylic acid (4-methoxy-7-morpholin-4-yl-benzothiazol-2-yl)-amide significantly reduced APEC-induced deficits in locomotor activity, confirming its efficacy as a selective A_{2A} receptor antagonist. * p<0.05 as determined by the Mann-Whitney test.

The antidepressant activity of 4-hydroxy-4-methyl-piperidine-1-carboxylic acid (4-methoxy-7-morpholin-4-yl-benzothiazol-2-yl)-amide was tested using three validated models of depression: the swim stress test stress-induced anhedonia test and the test of differential-reinforcement-of-low-rate (30 seconds) (DRL 30). All tests were performed on Wistar rats.

The swim stress test relies on the principle that when placed in water, rodents after an initial period of vigorous activity, adopt a characteristic immobile posture making only the minimal movements necessary to stay afloat. A reduction in the time of immobility is considered indicative of potential antidepressant-like properties of a particular drug.

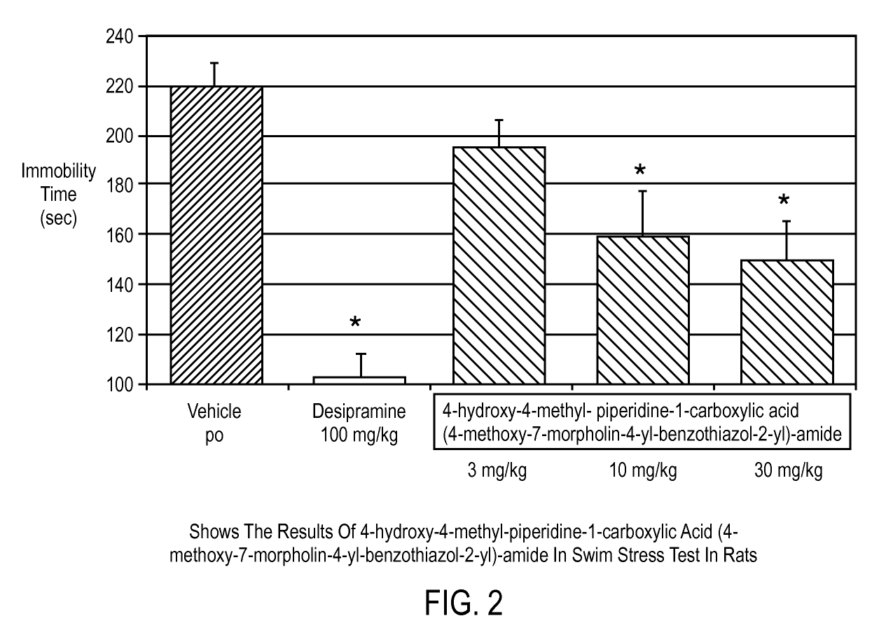

Administration of 4-hydroxy-4-methyl-piperidine-1-carboxylic acid (4-methoxy-7-morpholin-4-yl-benzothiazol-2-yl)-amide po to female rats significantly decreased the mean total duration of immobility in a swim stress test in a dose-dependent manner compared with controls (FIG. 2). A similar result was obtained with desipramine (100 mg/kg po), the TCA used as a reference drug.

Female Wistar rats received 4-hydroxy-4-methyl-piperidine-1-carboxylic acid (4-methoxy-7-morpholin-4-yl-benzothiazol-2-yl)-amide po at doses of 3, 10 and 30 mg/kg, 2 hours prior to the swim test. The tricyclic antidepressant desipramine was used as reference drug at 100 mg/kg po. Data are mean±SEM based on 8 animals per group. A dose-dependent decrease in the duration of immobility was observed with 4-hydroxy-4-methyl-piperidine-1-carboxylic acid (4-methoxy-7-morpholin-4-yl-benzothiazol-2-yl)-amide. *p<0.05 based on Student's t-test.

In the stress-induced anhedonia test, animals are implanted with an electrode in the area of the brain known to be involved in the feeling of reward or pleasure. This electrode allows the animals to stimulate themselves (self-stimulation behavior). The animals are then exposed sequentially for several weeks to a variety of mild, intermittent, and unpredictable stressors (i.e. confinement to restricted spaces, deprivation of food and/or water, reversed light/dark cycle). As a result, the threshold for self-stimulation becomes progressively elevated, meaning that the animals' sensitivity to reward progressively decreases. This is interpreted as the gradual development of anhedonia, the loss of interest or pleasure in daily activities, which is a hallmark of depression.

After being exposed for three weeks to a variety of mild, intermittent and unpredictable stressors, male rats were chronically treated with 4-hydroxy-4-methyl-piperidine-1-carboxylic acid (4-methoxy-7-morpholin-4-yl-benzothiazol-2-yl)-amide or vehicle by intraperitoneal (ip) injection once daily for three weeks. Self-stimulation behavior was recorded twice-weekly to follow development of stress-induced anhedonia, determined as the % variation in self-stimulation threshold (anhedonia index).

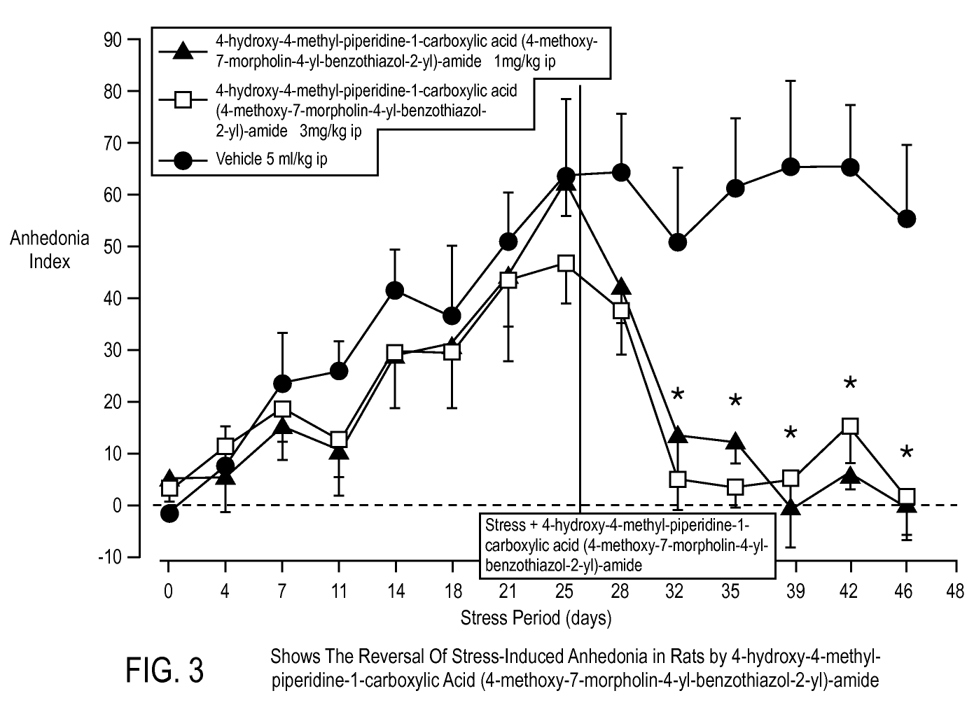

Animals treated with 4-hydroxy-4-methyl-piperidine-1-carboxylic acid (4-methoxy-7-morpholin-4-yl-benzothiazol-2-yl)-amide returned to normal levels of reward sensitivity after one to two weeks of treatment (FIG. 3) while vehicle-treated, stressed animals remained anhedonic for three weeks following testing. These results provide further evidence of the antidepressant properties of 4-hydroxy-4-methyl-piperidine-1-carboxylic acid (4-methoxy-7-morpholin-4-yl-benzothiazol-2-yl)-amide.

After being exposed to various mild, intermittent and unpredictable stressors for 3 weeks, anhedonic male Wistar rats were given 4-hydroxy-4-methyl-piperidine-1-carboxylic acid (4-methoxy-7-morpholin-4-yl-benzothiazol-2-yl)-amide ip daily at doses of 1 or 3 mg/kg for 3 weeks. Control animals were treated with vehicle only. Self-stimulation behavior was recorded twice weekly to follow the development of anhedonia, determined from the % variation in self-stimulation threshold (arbitrarily defined as 15 stimulation requests per minute=anhedonia index) Data are mean±SEM based on 7-8 animals per group. Administration of 4-hydroxy-4-methyl-piperidine-1-carboxylic acid (4-methoxy-7-morpholin-4-yl-benzothiazol-2-yl)-amide caused a significant reduction in the anhedonia index after 1-2 weeks compared with controls. *p<0.05 based on a three-way ANOVA followed by an unpaired t-test (FIG. 3).

The differential-reinforcement-of low-rate (DRL) test is used not only to assess the potential antidepressant properties of drugs but also their anxiolytic potential. Animals are trained to respond to a certain stimulus by pressing a lever. Typical antidepressants, such as TCAs, increase the time between responses, thereby decreasing the response rate. They also increase the number of reinforced responses i.e. repeated pressing of the lever at least 30 seconds after the initial pressing of the lever. Opposite effects are observed with atypical antidepressants such as nomifensine and with benzodiazepine-like anxiolytics.

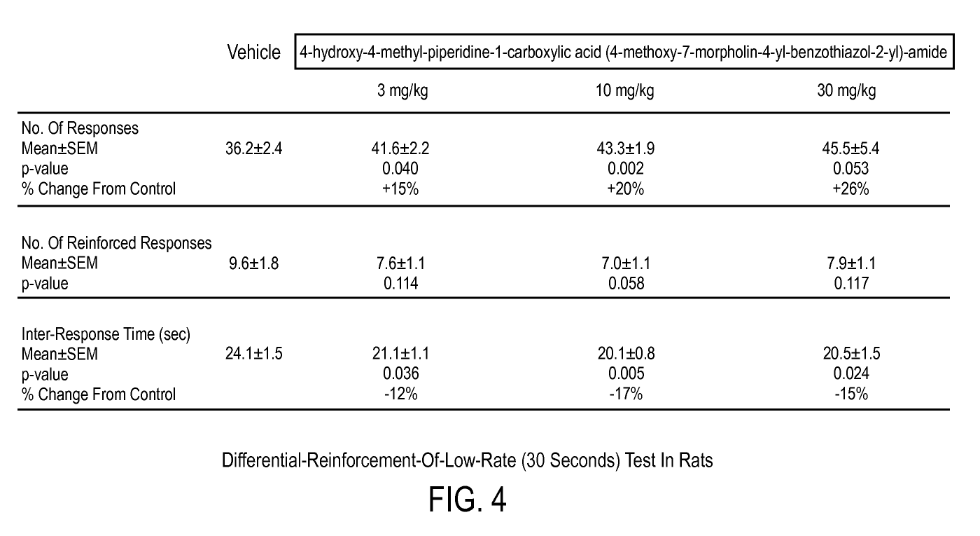

Administration of 4-hydroxy-4-methyl-piperidine-1-carboxylic acid (4-methoxy-7-morpholin-4-yl-benzothiazol-2-yl)-amide po to male rats caused an increase in total mean number of responses and a decrease in the mean time between responses that was dose-dependent (FIG. 4). 4-hydroxy-4-methyl-piperidine-1-carboxylic acid (4-methoxy-7-morpholin-4-yl-benzothiazol-2-yl)-amide was also associated with a decrease in the number of reinforced responses.

These data show that 4-hydroxy-4-methyl-piperidine-1-carboxylic acid (4-methoxy-7-morpholin-4-yl-benzothiazol-2-yl)-amide behaves like an atypical antidepressant and also exhibits anxiolytic properties.

The anxiolytic properties of 4-hydroxy-4-methyl-piperidine-1-carboxylic acid (4-methoxy-7-morpholin-4-yl-benzothiazol-2-yl)-amide were tested using the elevated plus-maze, widely used as an anxiety paradigm, and based on the natural aversion of rodents to open spaces and heights. Animals are placed in the center of an elevated maze containing two closed and two open arms. The time spent in the open arms of the maze, together with the number of times the animals enter the open arms of the maze, are taken as indices of the level of neophobic anxiety of these animals.

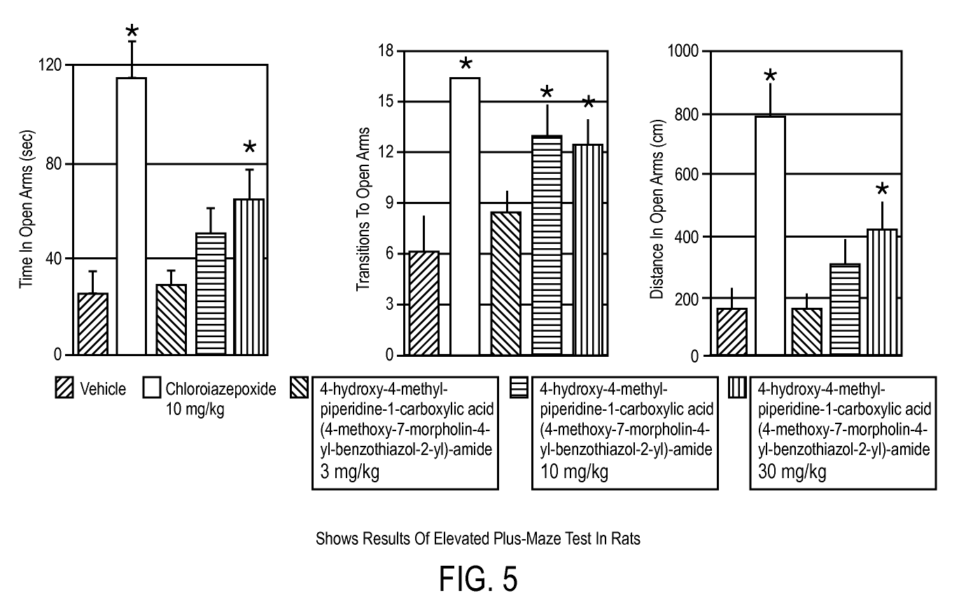

Administration of 4-hydroxy-4-methyl-piperidine-1-carboxylic acid (4-methoxy-7-morpholin-4-yl-benzothiazol-2-yl)-amide po to male rats resulted in a significant dose-dependent increase in the amount of time spent in the open arms of the elevated plus-maze as compared with controls (administered vehicle) (FIG. 5). The proportion of entries into the open arms and the distance traveled in open arms were also significantly increased following treatment with 4-hydroxy-4-methyl- piperidine-1-carboxylic acid (4-methoxy-7-morpholin-4-yl-benzothiazol-2-yl)-amide. A similar result was observed for chlordiazepoxide, used as a reference drug. This test, thus, confirms the anxiolytic-like properties of 4-hydroxy-4-methyl-piperidine-1-carboxylic acid (4-methoxy-7-morpholin-4-yl-benzothiazol-2-yl)-amide.

To generate the data shown in FIG. 5, Male Sprague-Dawley rats were administered either 4-hydroxy-4-methyl-piperidine-1-carboxylic acid (4-methoxy-7-morpholin-4-yl-benzothiazol-2-yl)-amide po at doses 3, 10 and 30 mg/kg, chlordiazepoxide po at 10 mg/kg or vehicle. Levels of anxiety were determined by the time spent and the distance traveled in the open arms of the maze. Data are mean±SEM based on 12 animals per group. 4-hydroxy-4-methyl-piperidine-1-carboxylic acid (4-methoxy-7-morpholin-4-yl-benzothiazol-2-yl)-amide caused a dose-dependent increase in the amount of time spent in the open arms of the maze, in the number of transitions into the open arms and the distance traveled in the open arms, thus confirming its anxiolytic-like potential. *p<0.05 determined by ANOVA followed by a Bonferroni test. FIG. 5: SEM: standard error of the mean. Results are based on 11 animals. Only animals demonstrating previous stable performance in the DRL 30 test were used. Each animal was used at its own control and received all doses of 4-hydroxy-4-methyl-piperidine-1-carboxylic acid (4-methoxy-7-morpholin-4-yl-benzothiazol-2-yl)-amide tested (3, 10 and 30 mg/kg) or vehicle. Data analyzed with a paired t-test. 4-hydroxy-4-methyl-piperidine-1-carboxylic acid (4-methoxy-7-morpholin-4-yl-benzothiazol-2-yl)-amide behaves like anxiolytics or atypical antidepressants, causing a dose-dependent increase in total mean number of responses and a decrease in the mean time between responses.

The passive avoidance test in rats was used to evaluate the potential cognitive-enhancing effects of 4-hydroxy-4-methyl-piperidine-1-carboxylic acid (4-methoxy-7-morpholin-4-yl-benzothiazol-2-yl)-amide. This test relies on training rodents to avoid an aversive event (electric shock) by curbing a normal behavior, and at specified intervals after training, testing the animals for retention of such learning.

Adult rats were trained in a step-down test to avoid foot shock by remaining on a plastic platform. 4-hydroxy-4-methyl-piperidine-1-carboxylic acid (4-methoxy-7-morpholin-4-yl-benzothiazol-2-yl)-amide was given po immediately post-training. Amnesia was induced by administering scopolamine subcutaneously (sc) given immediately post-training.

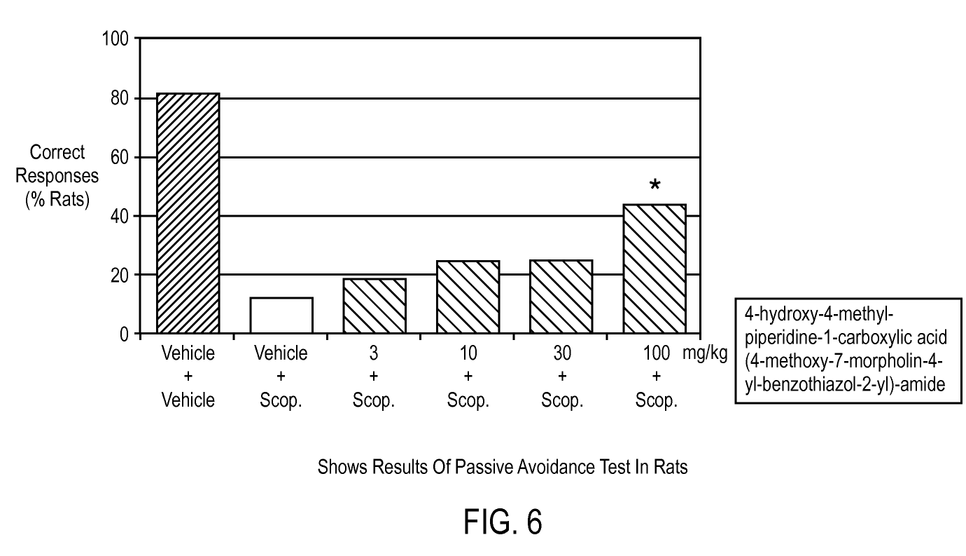

Administration of 4-hydroxy-4-methyl-piperidine-1-carboxylic acid (4-methoxy-7-morpholin-4-yl-benzothiazol-2-yl)-amide resulted in a reversal of the scopolamine-induced retention deficit that was dose-dependent and statistically significant at the highest dose tested (100 mg/kg po) (FIG. 6). Thus, these data indicate that 4-hydroxy-4-methyl-piperidine-1-carboxylic acid (4-methoxy-7-morpholin-4-yl-benzothiazol-2-yl)-amide exhibits cognitive-enhancing-like properties in the passive avoidance test in rats.

The protocol to generate FIG. 6 data are as follows: Adult rats (n=16/dose group) were trained to avoid foot shock by remaining on a plastic platform in a step-down test. Immediately post-training, they received 4-hydroxy-4-methyl-piperidine-1-carboxylic acid (4-methoxy-7-morpholin-4-yl-benzothiazol-2-yl)-amide po at doses of 3, 10, 30 and 100 mg/kg, or vehicle. Amnesia was induced by administration of scopolamine sc at 1 mg/kg immediately post-training. Retention, measured as the % of rats with correct responses, was assessed 2 h post-training. 4-hydroxy-4-methyl-piperidine-1-carboxylic acid (4-methoxy-7-morpholin-4-yl-benzothiazol-2-yl)-amide caused a dose-dependent reversal of the scopolamine-induced retention deficit, suggesting cognitive-enhancing properties of the compound. *p<0.05 one-tailed chi-square test.

### Example 6

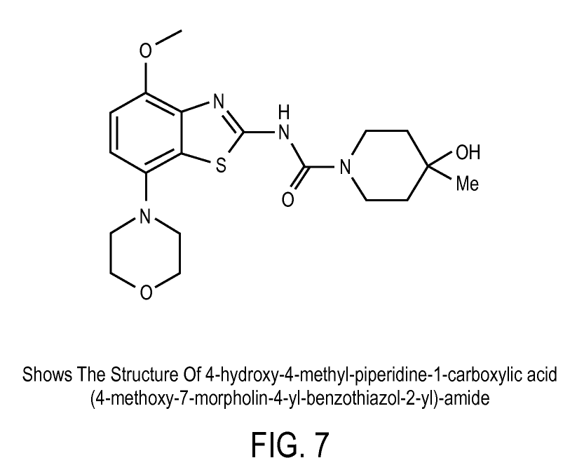

These studies were conducted to investigate the in-vitro pharmacological profile of 4-hydroxy-4-methyl-piperidine-1-carboxylic acid (4-methoxy-7-morpholin-4-yl-benzothiazol-2-yl)-amide with respect to its affinity for the A_{2A} receptor and it's selectivity profile (other adenosine receptors from various species and CEREP profile). In addition, the ability of 4-hydroxy-4-methyl-piperidine-1-carboxylic acid (4-methoxy-7-morpholin-4-yl-benzothiazol-2-yl)-amide to block agonist activation at the A_{2A} (and A₁ receptor for selectivity) was assessed. FIG. 7 shows the structure of 4-hydroxy-4-methyl-piperidine-1-carboxylic acid (4-methoxy-7-morpholin-4-yl-benzothiazol-2-yl)-amide.

4-hydroxy-4-methyl-piperidine-1-carboxylic acid (4-methoxy-7-morpholin-4-yl-benzothiazol-2-yl)-amide was initially dissolved in DMSO at a concentration of 20 mM. Subsequent dilutions were made in assay buffer with a maximum final concentration of 1.25% DMSO in radioligand binding assays and 0.3% for FLIPR assays. There was no effect of DMSO on the radioligand binding or FLIPR assays over the range of concentrations used in the assay.

Recombinant adenosine A₁ (human, hA1; rat rA1), A_{2A} (human hA2A; rat rA2A), A_{2B} (human hA2B), and A₃ (human hA3, dog dA3) were expressed in chinese hamster ovary (CHO) cells using the semliki forest virus expression system. Rat A₃ (rA3) membranes were purchased from Receptor Biology Inc. (USA). Dog (Beagle, male) A₁ (dA1) and A_{2A} (dA2A) receptors brain tissue was obtained and the cortical (dA₁) and striatal (dA_{2A}) tissue was dissected and frozen at −80° C. until membrane preparation. Monkey (Saimiri, male) brain striatal (mA₁ and mA_{2A}) and cortical (mA₃) tissue were dissected and frozen at −80° C. until membrane preparation. The various receptor cell pellets or animal tissue dissected regions, were prepared by homogenizing (Polytron) the pellet/tissue in homogenization buffer (50 mM Tris-CL pH 7.4, 10 mM EDTA), then centrifuging the resulting suspension at 47800 g for 15 min at 4° C. The pellet was re-suspended in homogenizing buffer, and subsequently re-centrifuged (same conditions). The pellet was re-suspended in buffer (10 mM Tris-Cl, EDTA 2 mM pH 7.4 and adenosine deaminase 0.5 U/ml), incubated at 37° C. for 15 min, then re-centrifuged. The resulting pellet was re-suspended in Tris 10 mM, EDTA 2 mM and 10% Sucrose. The concentration of protein was determined, and the membranes were aliquoted and stored at −80° C. until further use.

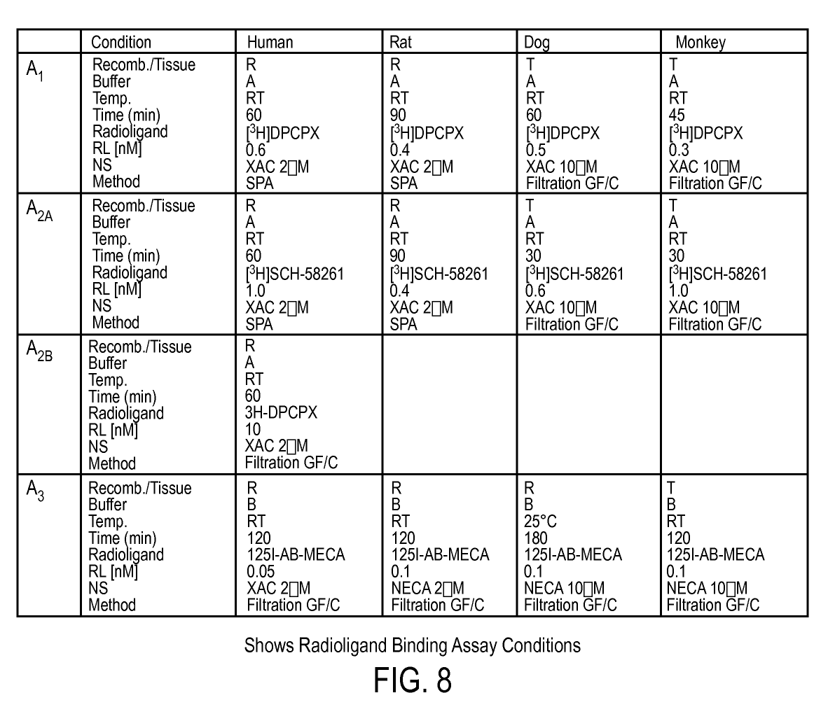

All radioligand binding assays were carried out in 96-well plates in the presence of radioligand (FIG. 8) and 10 concentrations of 4-hydroxy-4-methyl-piperidine-1-carboxylic acid (4-methoxy-7-morpholin-4-yl-benzothiazol-2-yl)-amide (ranging from 10 μM-0.03 nM). Dilutions of 4-hydroxy-4-methyl-piperidine-1-carboxylic acid (4-methoxy-7-morpholin-4-yl-benzothiazol-2-yl)-amide were made using a Beckman Biomek 2000 laboratory automation workstation, in assay buffer. Non-specific binding was defined using xanthine amine congener or NECA. Each well contained membrane protein (varying concentrations), 0.5 mg of Ysi-poly-1-lysine SPA beads (for all SPA assays not filtration see FIG. 8) and 0.1 U adenosine deaminase in a final volume of 200 μl of buffer A or B (containing for A: 50 mM Tris, 120 mM NaCl, 5 mM KCl, 2 mM CaCl₂ and 10 mM MgCl₂ (pH 7.4); for B: 50 mM Tris, 1 mM EDTA and 10 mM MgCl₂ (pH 7.4)). All assays were conducted in duplicate and repeated at least two times. Assay plates were incubated for varying times at room temperature before centrifugation (SPA) or filtration (see FIG. 8). For filtration assays these were terminated by rapid filtration under vacuum through GF/C filters, presoaked for at least 30 min with PEI (polyethylenimine; 0.3%), with 5×0.4 ml washes of ice-cold Tris buffer (50 mM, pH 7.4). For both SPA and filtration plates, bound ligand was determined using a Packard Topcount scintillation counter.

The CPM value for each duplicate of a concentration of competing compound was averaged (y1) then the % specific binding calculated, (((y1—non-specific)/(total binding non-specific))×100). Graphs were plotted with the % specific binding using XLfit, a curve fitting program that iteratively plots the data using Levenburg Marquardt algorithm. The single site competition analysis equation used was y=A+((BA)/(1+((x/C)D))), where y is the % specific binding, A is the minimum y, B is the maximum y, C is the IC50, x is the log₁₀ of the concentration of the competing compound and D is the slope of the curve (the Hill Coefficient). From these curves the IC50 (inhibition concentration at which 50% specific binding of the radioligand was displaced) and Hill coefficient were determined. The affinity constant (Ki) was calculated using the Cheng-Prussoff equation Ki=(IC50/1+([L]/Kd), where [L] is the concentration of radioligand and Kd is the affinity constant of the radioligand. The Ki was also expressed logarithmically as a pKi. For FIG. 8, the legend is R (recombinant); T (tissue); RT (room temperature); buffer A: 50 mM Tris, 120 mM NaCl, 5 mM KCl, 2 mM CaCl2 and 10 mM MgCl₂ (pH 7.4); buffer B: 50 mM Tris, 1 mM EDTA and 10 mM MgCl₂ (pH 7.4); RL (radioligand); NS (non-specific binding); SPA (scintillation proximity assay).

CHO cells stably expressing the promiscuous G-protein G_{α16} were transfected with the human plasmids encoding either the human A₁ or A_{2A} receptors. Stable cell lines were selected based on functional responses detected in FLIPR. Stable cells were cloned by limited dilution to yield monoclonal cell lines stably expressing G_{α16} and either the human A₁ (clone 12) or A_{2A} (clone 34) receptor.

The stable cells lines were grown in Dulbecco's Modified Eagles medium (DMEM) containing 10% heat inactivated foetal bovine serum (FBS), 1% penicillin-streptomycin, 1% L-glutamate, 1% essential amino acids at 37° C. in a 10% CO₂ incubator at 95% humidity.

On the afternoon before assay, cells were plated at a density of 50,000 cells/well into black 96 well plates with clear bottoms to allow cell inspection and fluorescence measurements from the bottom of each well. The density of cells was sufficient to yield a confluent monolayer the next day. Hanks balanced salt solution, without phenol red, containing 20 mM HEPES (pH 7.3) and 2.5 mM probenecid (assay buffer) was prepared fresh for each experiment. Dilutions were made using a Beckman Biomek 2000 laboratory automation workstation, in assay buffer. The dye-loading buffer consisted of a final concentration of 2 μM Fluo-4-AM (dissolved in DMSO and pluronic acid) in assay buffer. The existing maintenance media was removed from the wells and 100 μl of the dye-loading buffer was added to each well and incubated for approximately 60 min at 37° C. in a 5% CO₂ incubator at 95% humidity. Once dye-loaded, the cells were washed thoroughly on an Embla cell washer with the assay buffer to remove any unincorporated dye. Exactly 100 μl assay buffer was left in each well.

Assay conditions were as described previously by Porter et al., Brit. J. Pharmacol. 128, 13-20, 1999 and Patel et al., Brit. J. Pharm. 138, 671-677, 2003. Briefly, each 96 well plate containing dye-loaded cells was placed into the FLIPR drawer and the laser intensity set to a suitable level (to obtain basal values of approximately 10,000 fluorescence units). Agonist additions were made 10 s into the fluorescent measurements. For antagonist studies, cells were pre-incubated for 10 mins prior to experiments. The maximum fluorescent signal obtained was recorded and normalised to a positive control of 10 μM NECA performed in duplicate on every plate. Each 96 well plate contained two wells dedicated to the positive control (10 μM NECA) and two wells as a negative control (assay buffer alone). For pharmacological characterisation, all data were normalised to the positive control wells, which were expressed as 100% signal. Each agonist concentration-response curve was constructed using a four parameter logistic equation from Microsoft Excel XLFit as follows: Y=Minimum+((Maximum−Minimum)/(1+10^{(LogEC50-X)nH})). The efficacy of the compound was determined from the maximal value. The concentration of agonist that produced a half-maximal response is represented by the EC₅₀ value, the logarithm of which yielded the pEC₅₀ value. The single site competition analysis equation used was y=A+((B−A)/(1+((x/C)D))), where y is the % specific binding, A is the minimum y, B is the maximum y, C is the IC50 (concentration at which 50% inhibition of agonist stimulation), x is the log₁₀ of the concentration of the competing compound and D is the slope of the curve (the Hill Coefficient).

Further radioligand binding studies were conducted to assess the selectivity of 4-hydroxy-4-methyl-piperidine-1-carboxylic acid (4-methoxy-7-morpholin-4-yl-benzothiazol-2-yl)-amide over more than 67 receptors, neurotransmitter transporters and ion channels. In addition, Cellular biology assays were conducted on 16 enzyme targets to assess the selectivity of 4-hydroxy-4-methyl-piperidine-1-carboxylic acid (4-methoxy-7-morpholin-4-yl-benzothiazol-2-yl)-amide.

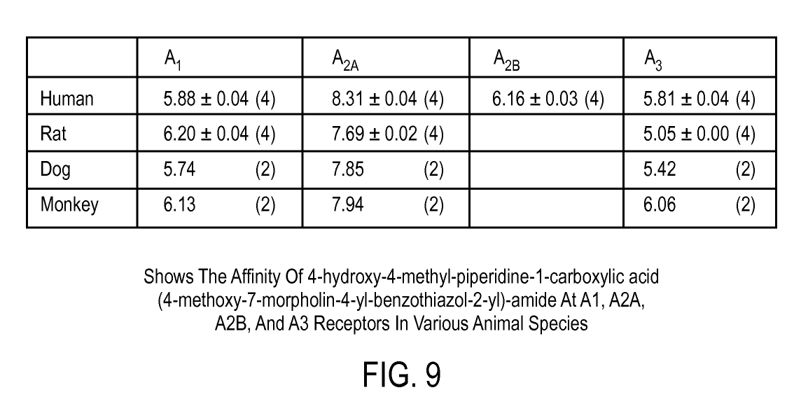

4-hydroxy-4-methyl-piperidine-1-carboxylic acid (4-methoxy-7-morpholin-4-yl-benzothiazol-2-yl)-amide has a high affinity for the human A_{2A} receptor (Ki 5±0.5 nM; pKi 8.31±0.04) with approximately 270, 140 and 314-fold selectivity compared to hA₁ (Ki: 1332±106 nM; pKi: 5.88±0.04), hA_{2B} (Ki: 700±55; pKi 6.16±0.03), and hA₃ (Ki: 1572±134 nM; pKi: 5.81±0.04) receptors, respectively for data including raw dpm, IC50, Ki, pKi and Hill coefficient determination). The Hill coefficient for each assay indicated that a single homogenous population of binding sites was being labelled. Binding studies in different species have also demonstrated that 4-hydroxy-4-methyl-piperidine-1-carboxylic acid (4-methoxy-7-morpholin-4-yl-benzothiazol-2-yl)-amide has a high affinity for A2A receptors in the rat (pKi 7.7), dog (pKi 7.9) and in the monkey (pKi 7.9) with good selectivity over the same species receptors (see FIG. 9).

Data represents the pKi±SEM (n), where the pKi is the Log10 of the affinity constant (Ki), and SEM is the standard error of mean (when n>2), and n is the number of assays. Details of the individual data are given in appendix 1 in section 6.1.

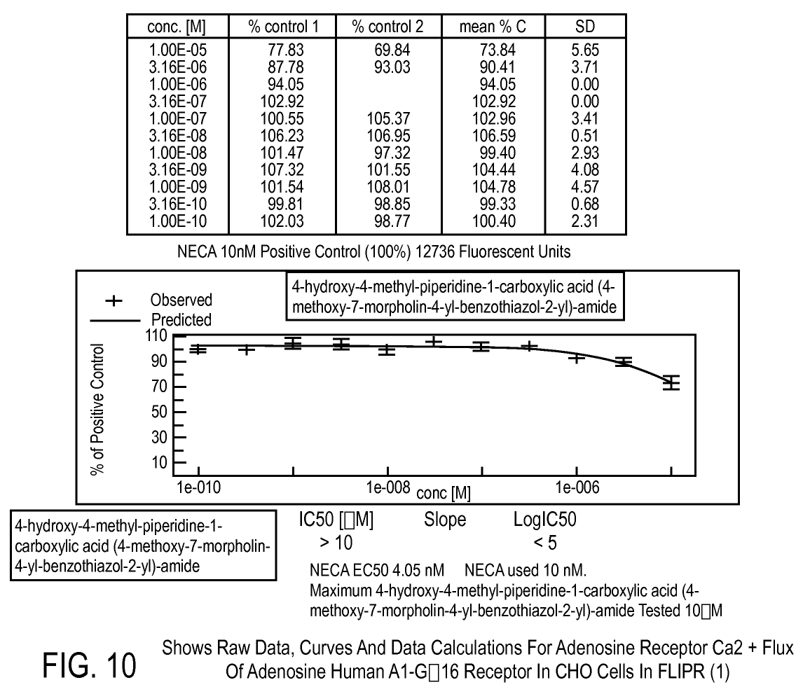

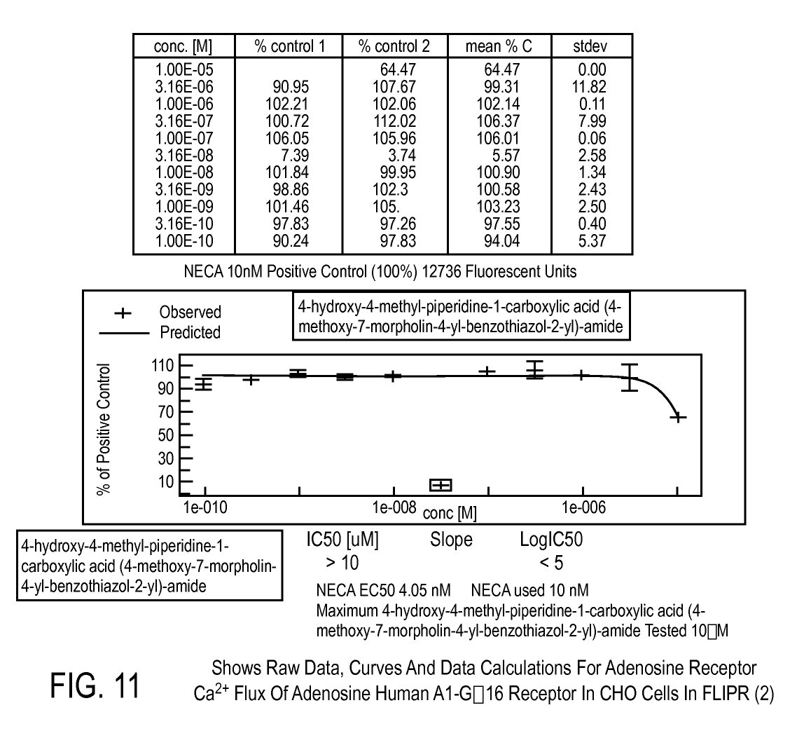

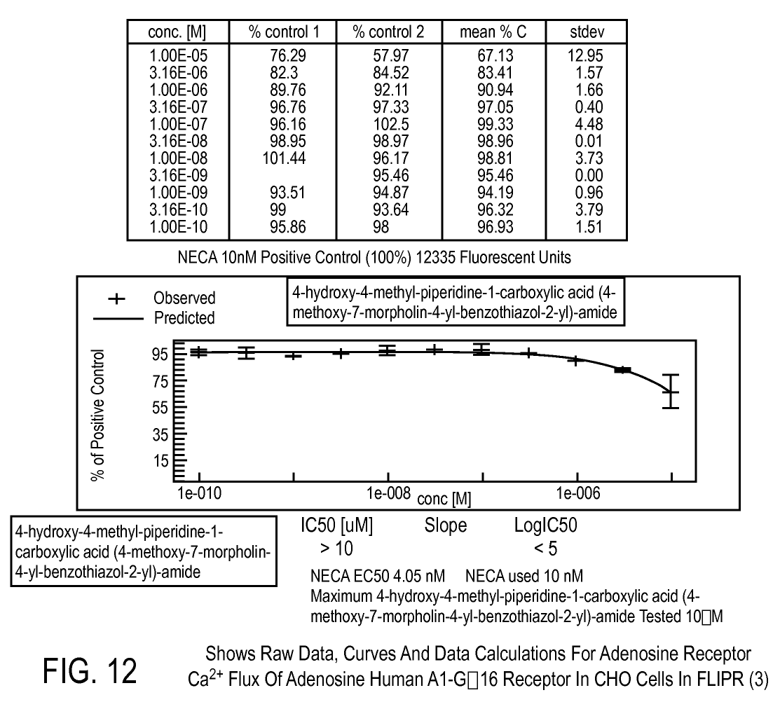

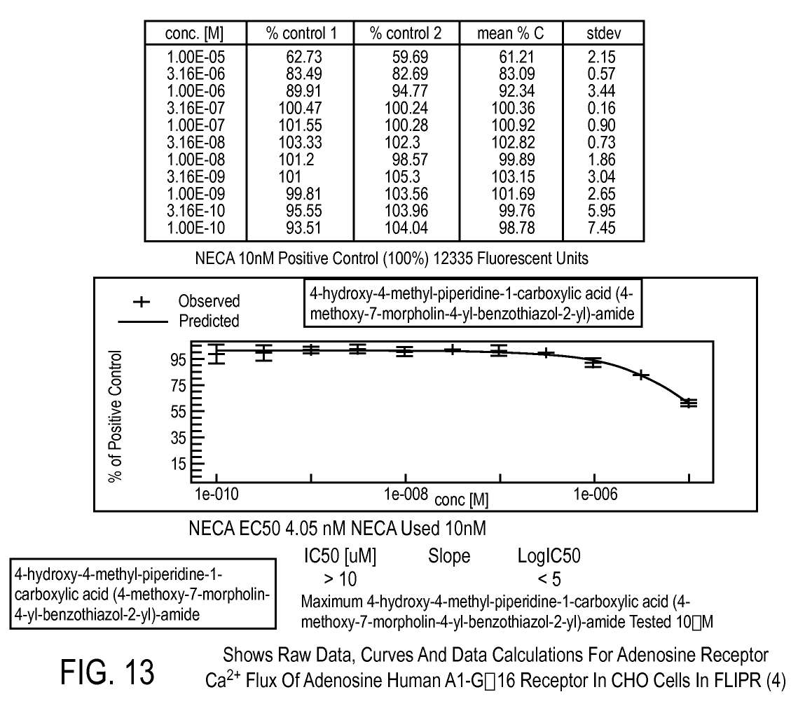

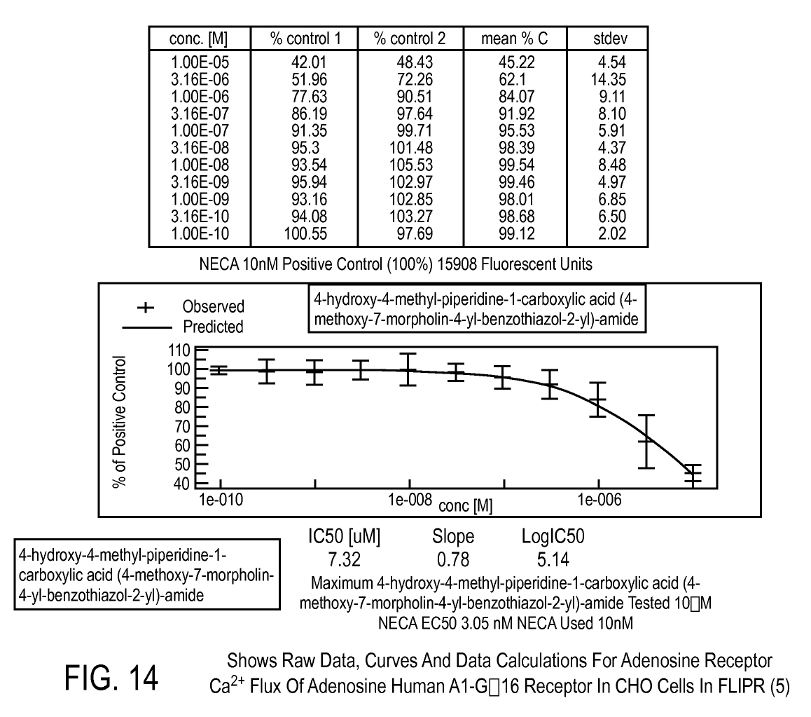

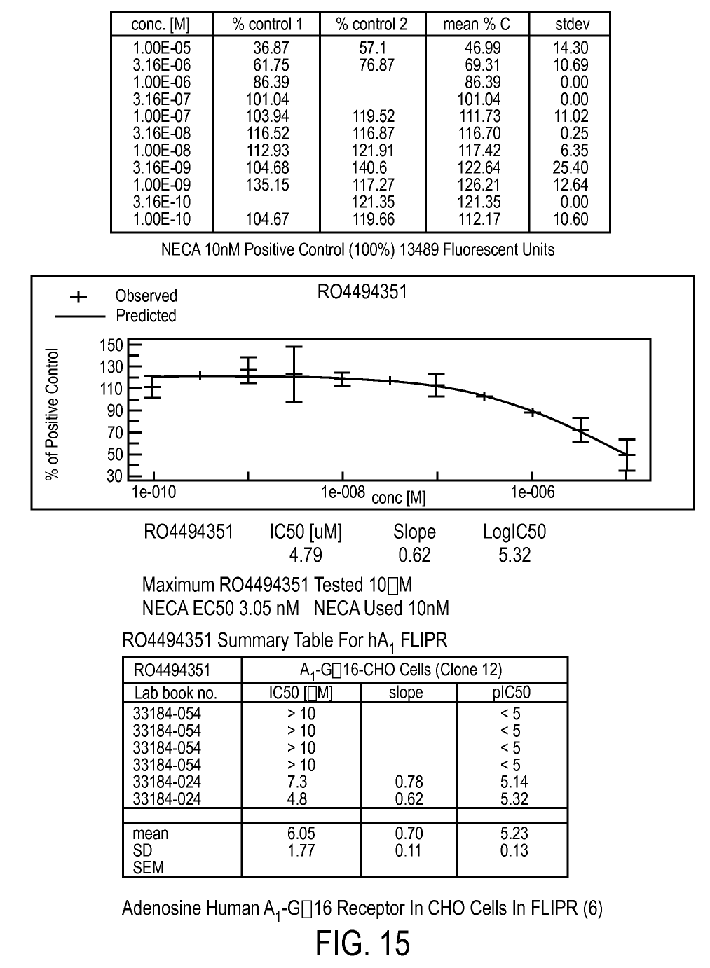

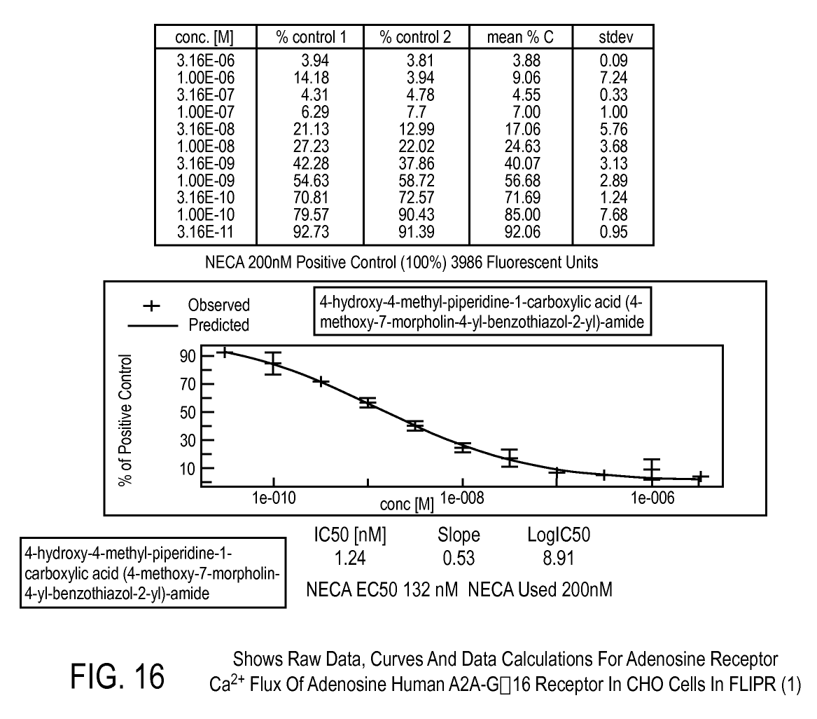

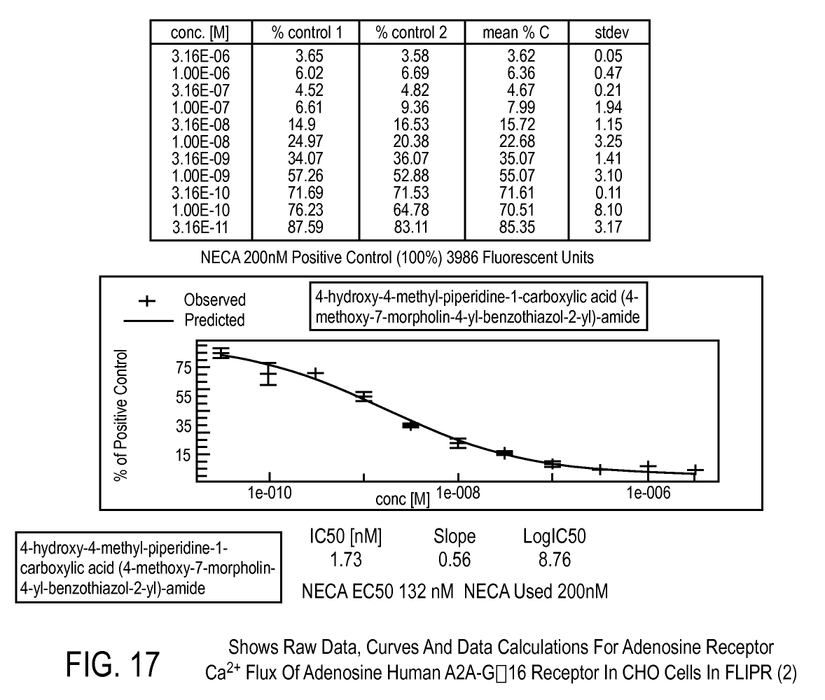

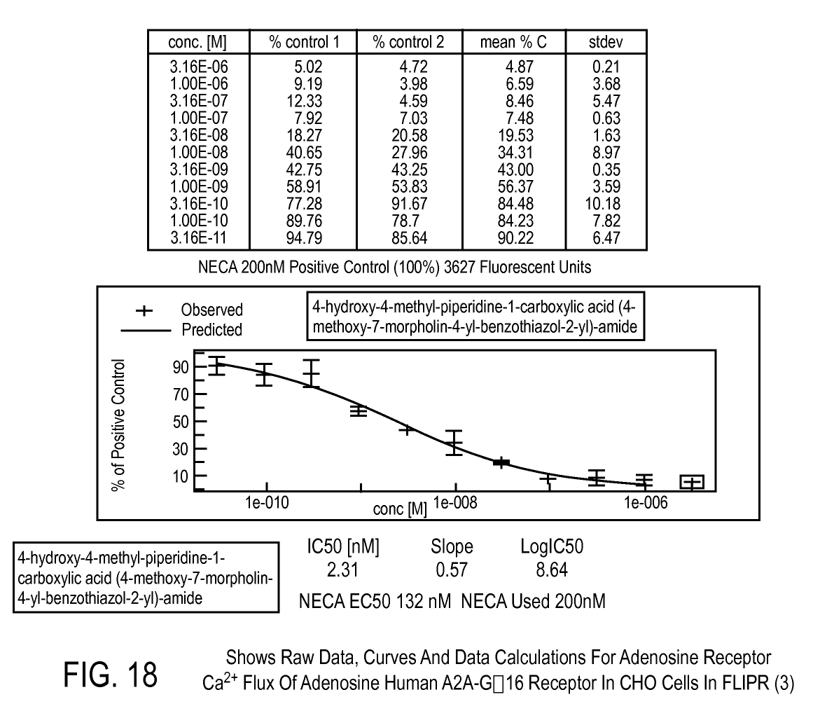

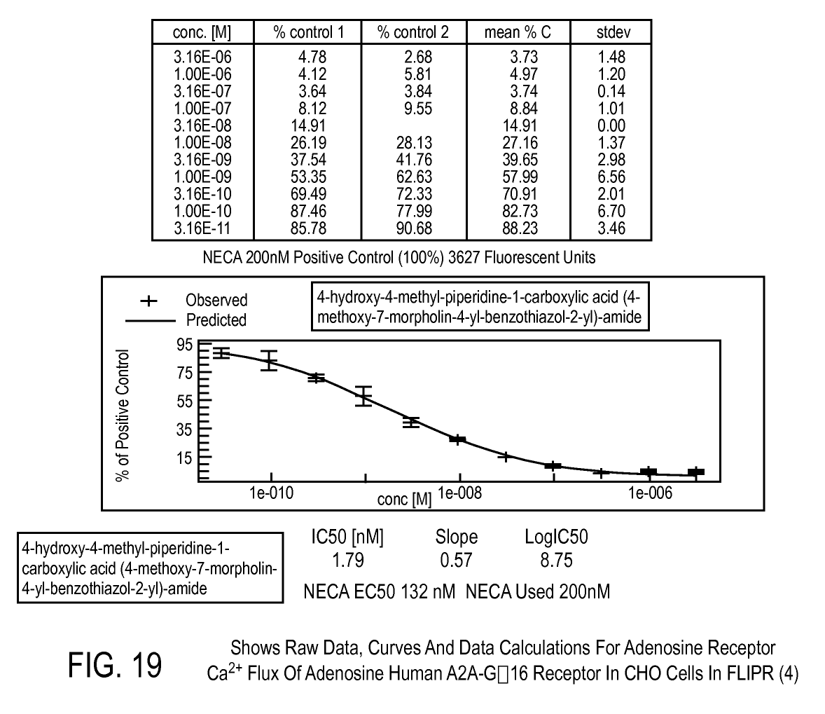

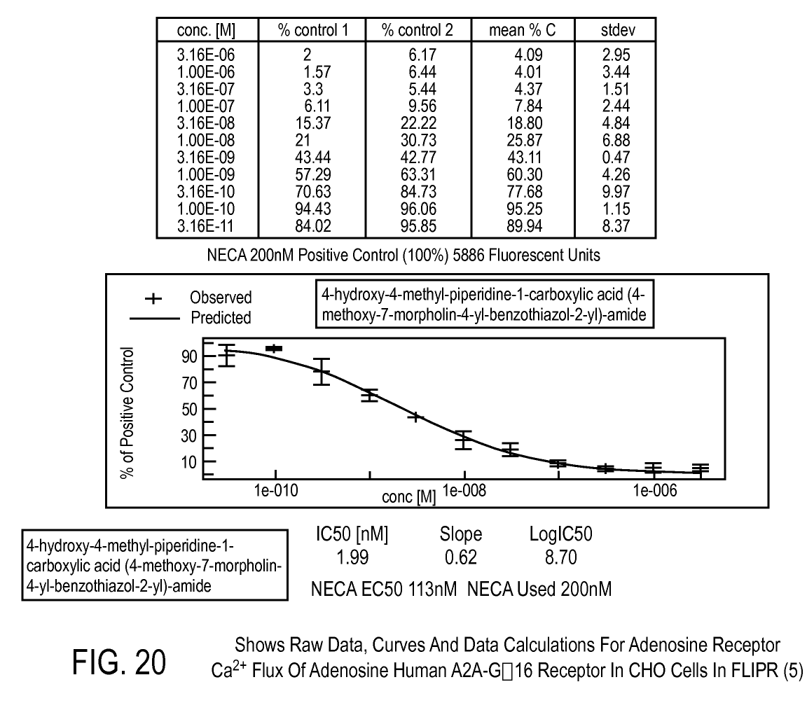

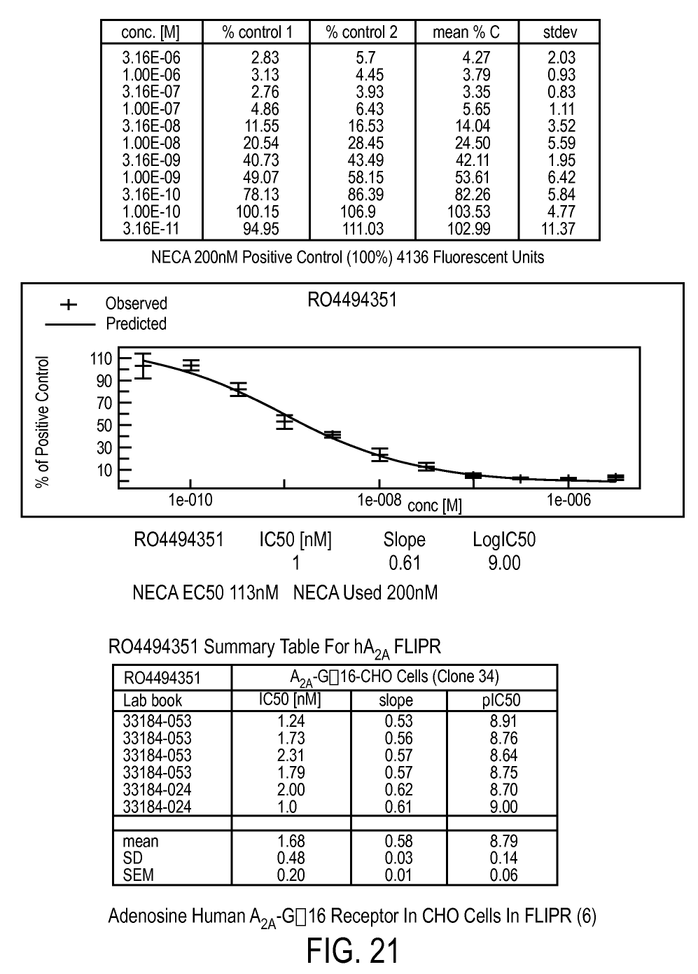

A functional assay assessed the ability of 4-hydroxy-4-methyl-piperidine-1-carboxylic acid (4-methoxy-7-morpholin-4-yl-benzothiazol-2-yl)-amide to antagonize the NECA stimulated (a non-specific adenosine receptor agonist) Ca²⁺ flux in hA_{2A}-Gα16-CHO cells. 4-hydroxy-4-methyl-piperidine-1-carboxylic acid (4-methoxy-7-morpholin-4-yl-benzothiazol-2-yl)-amide inhibited A_{2A}-mediated responses with a pIC₅₀ of 8.79±0.06 (Hill slope 0.6). 4-hydroxy-4-methyl-piperidine-1-carboxylic acid (4-methoxy-7-morpholin-4-yl-benzothiazol-2-yl)-amide antagonized the NECA-stimulated Ca²⁺ flux in hA₁-Gα16-CHO cells with a pIC₅₀ of between 5.22 or <5. Although the hA₁ antagonism was on the limit of detection, the data indicate that in this functional assay 4-hydroxy-4-methyl-piperidine-1-carboxylic acid (4-methoxy-7-morpholin-4-yl-benzothiazol-2-yl)-amide exhibited >4000 fold selectivity for the hA_{2A} receptor over hA₁ (see FIG. 10-21).

Further radioligand binding studies assessed the selectivity of 4-hydroxy-4-methyl-piperidine-1-carboxylic acid (4-methoxy-7-morpholin-4-yl-benzothiazol-2-yl)-amide over more than 67 receptors, neurotransmitter transporters and ion channels. 4-hydroxy-4-methyl-piperidine-1-carboxylic acid (4-methoxy-7-morpholin-4-yl-benzothiazol-2-yl)-amide had 1900-fold selectivity for the A_{2A} receptor over the targets tested with the exception of the adenosine transporter, where 4-hydroxy-4-methyl-piperidine-1-carboxylic acid (4-methoxy-7-morpholin-4-yl-benzothiazol-2-yl)-amide showed 55% displacement at 10 μM. 4-hydroxy-4-methyl-piperidine-1-carboxylic acid (4-methoxy-7-morpholin-4-yl-benzothiazol-2-yl)-amide showed a 1900-fold selectivity for the A_{2A} receptor over these targets, although 88% inhibition was seen at 10 μM for the phosphodiesterase (IV) enzyme.

4-hydroxy-4-methyl-piperidine-1-carboxylic acid (4-methoxy-7-morpholin-4-yl-benzothiazol-2-yl)-amide is a potent A_{2A} receptor antagonist with an excellent selectivity profile.

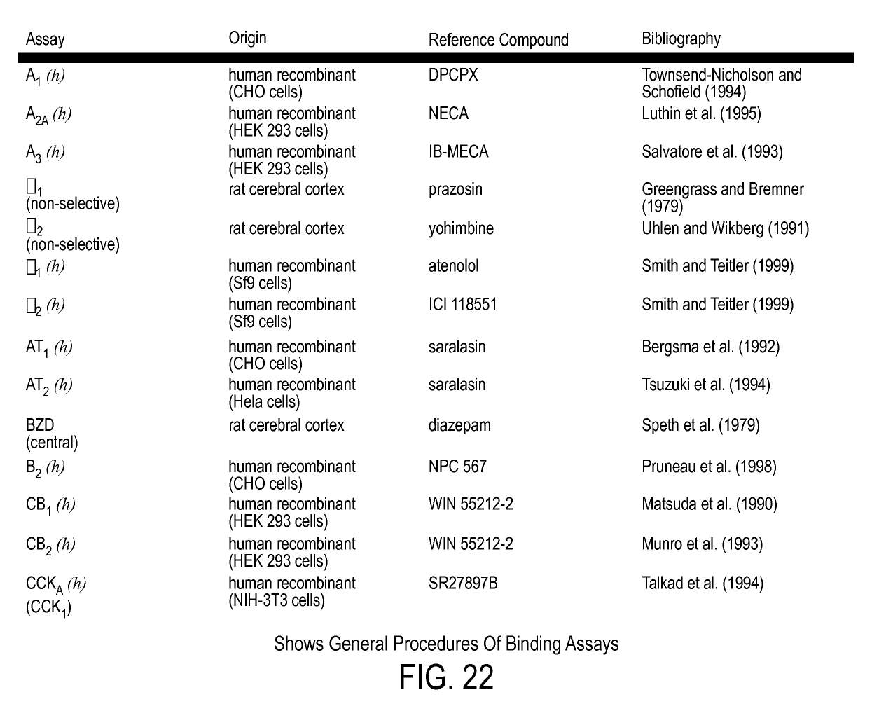

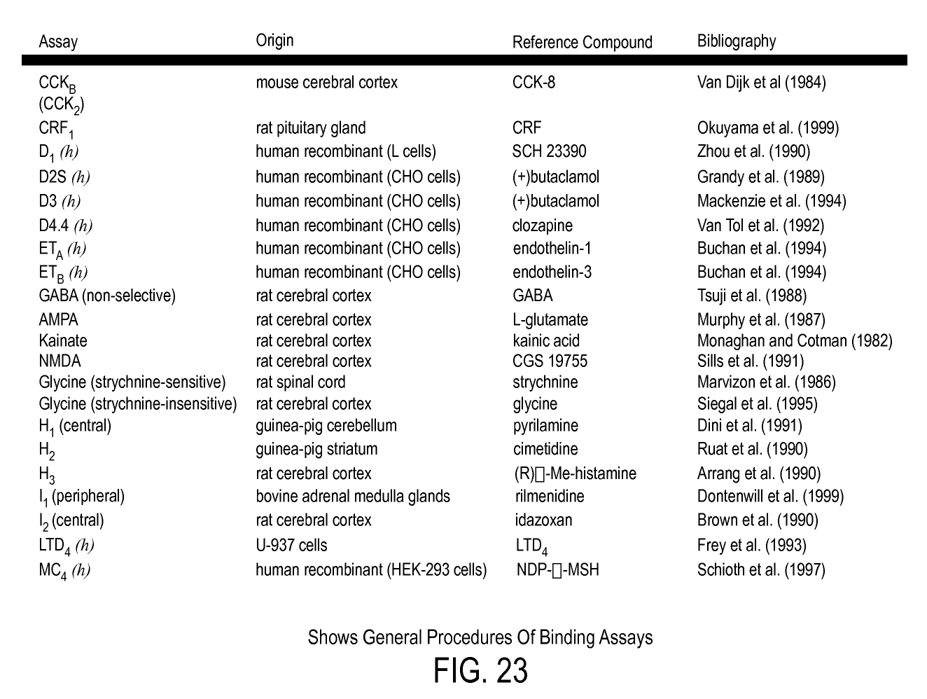

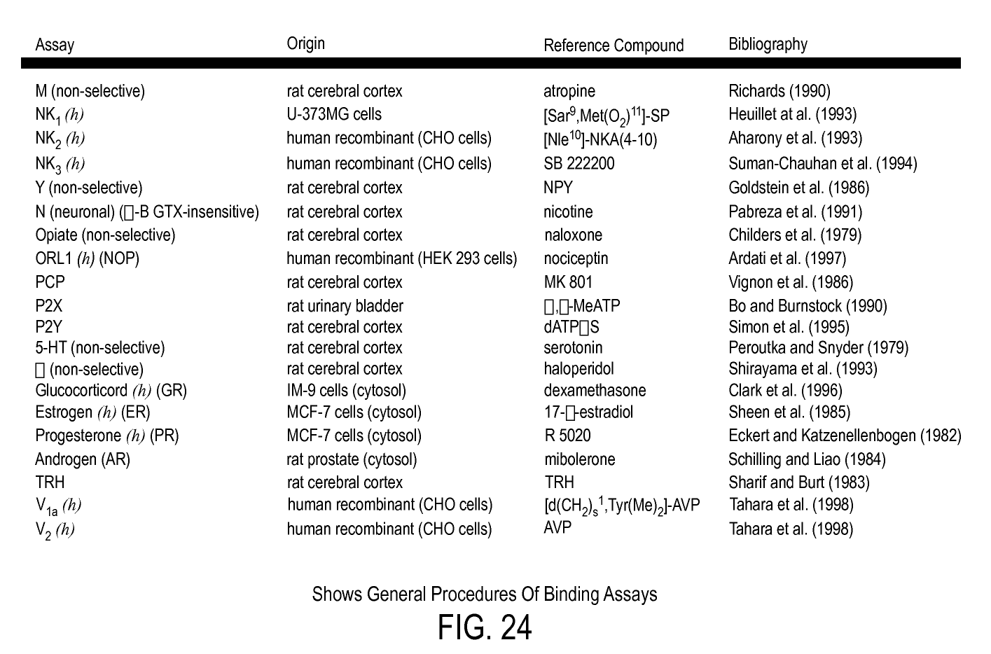

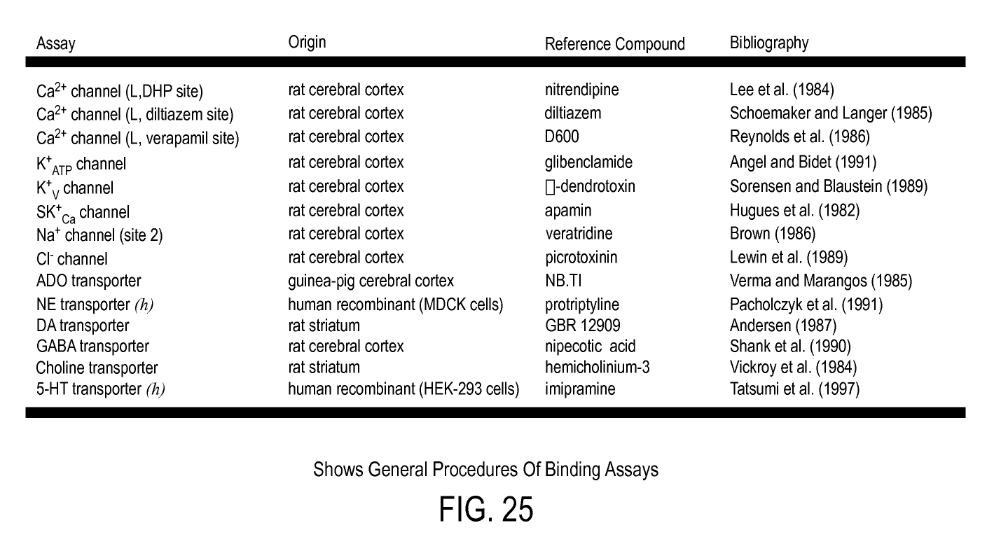

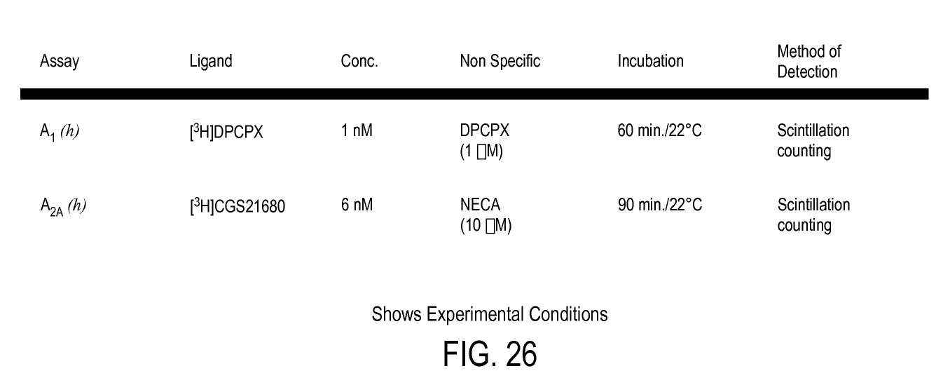

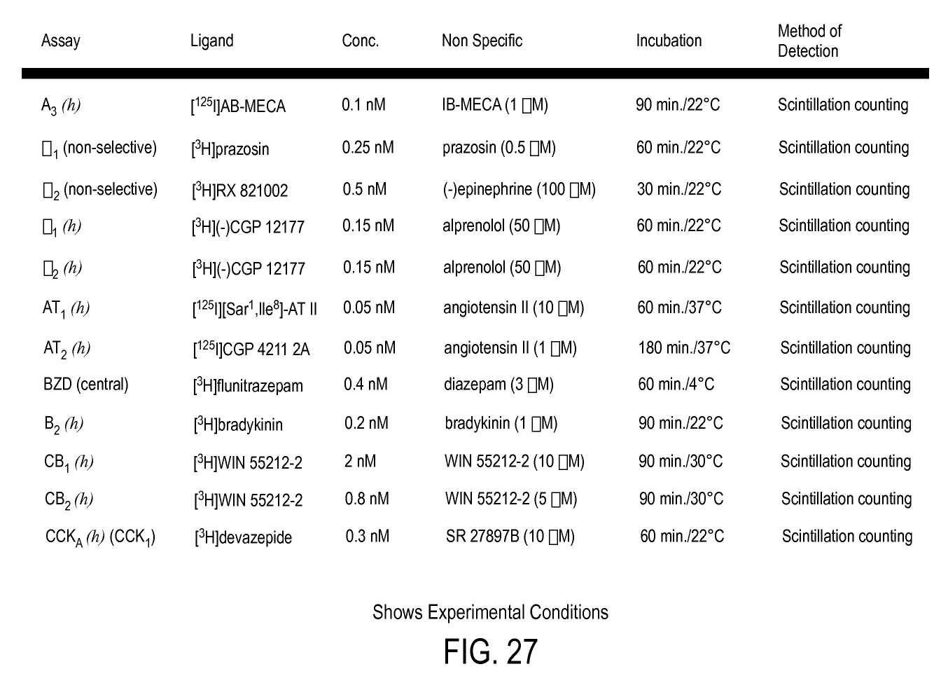

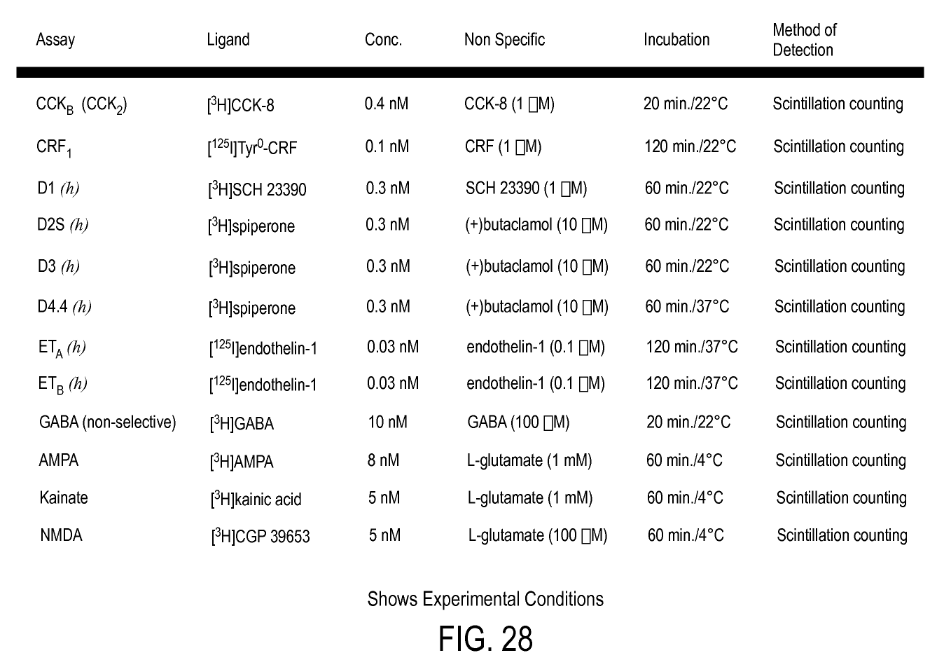

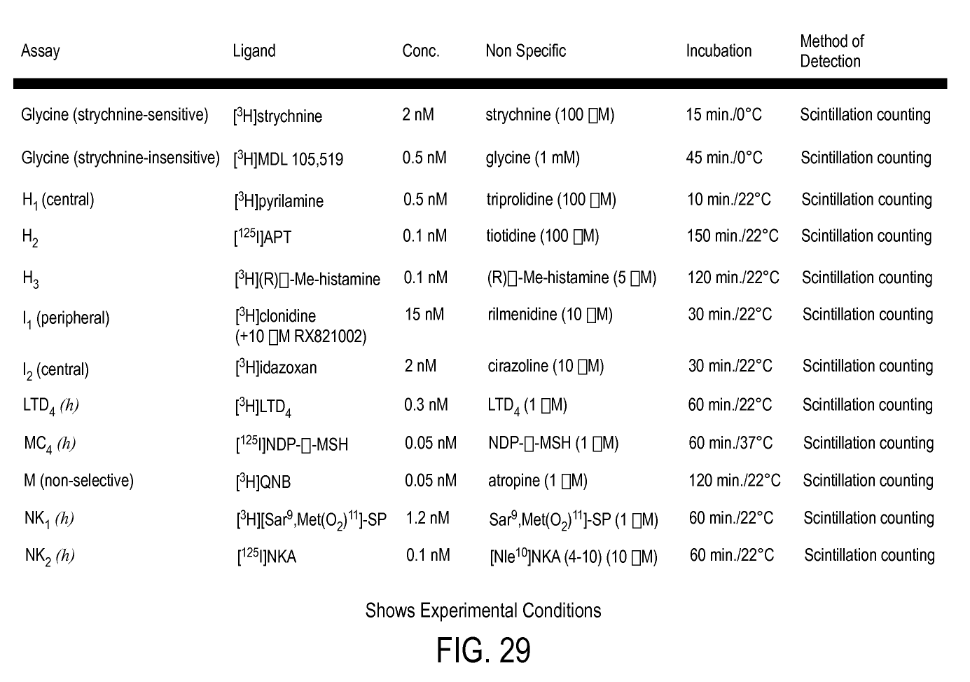

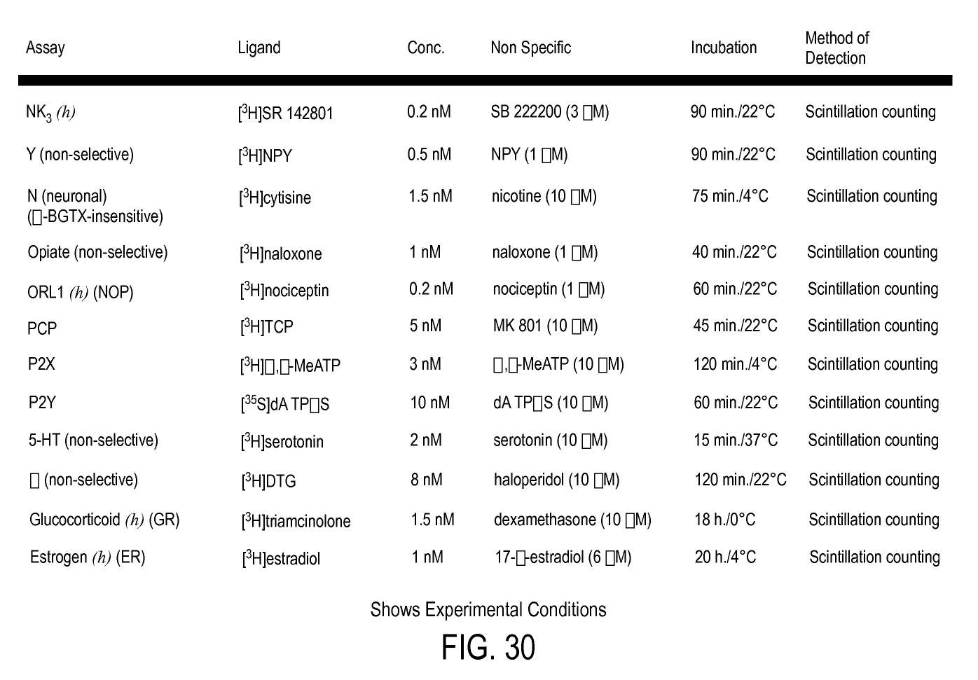

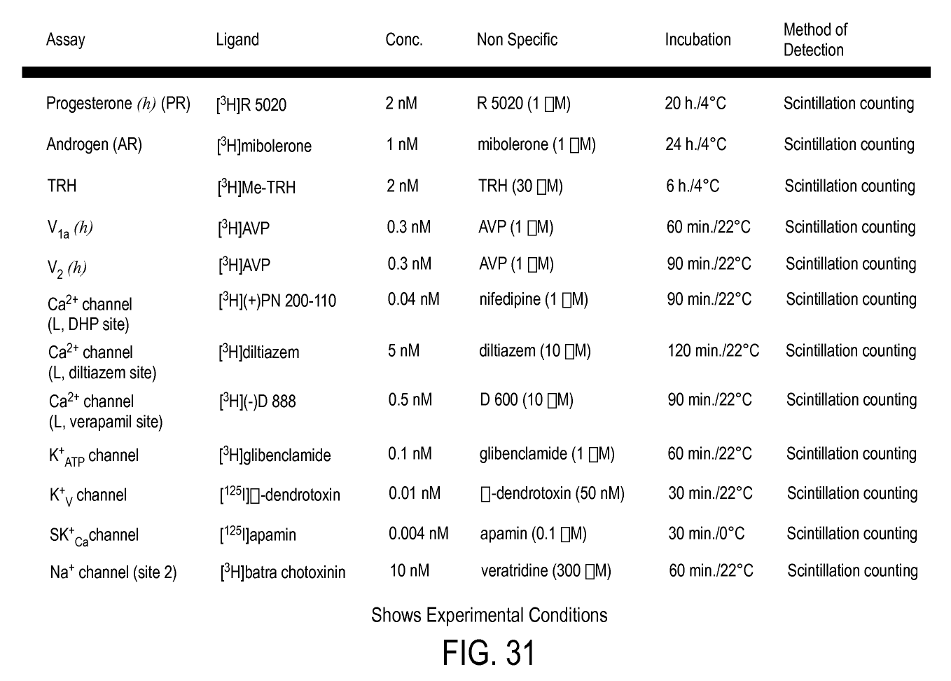

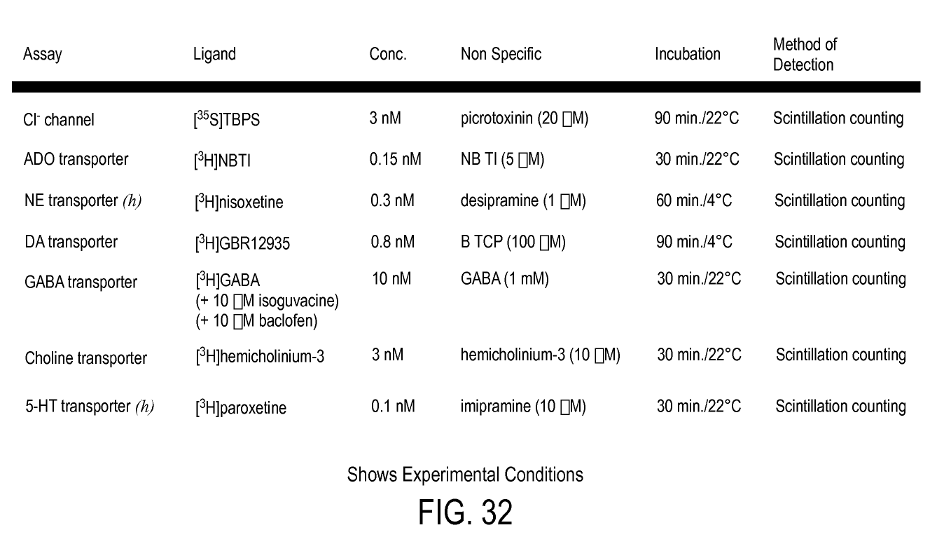

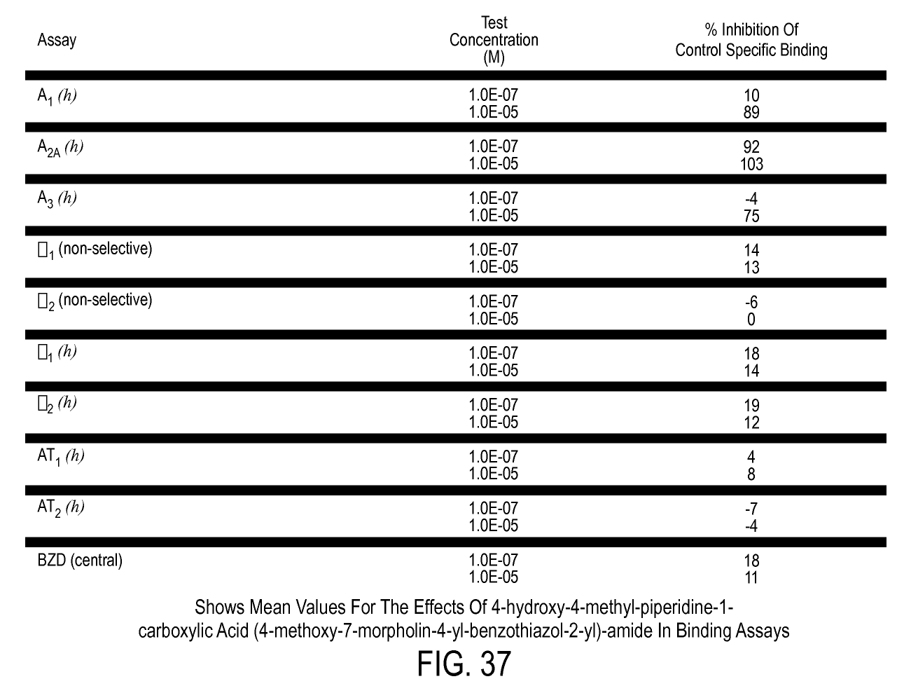

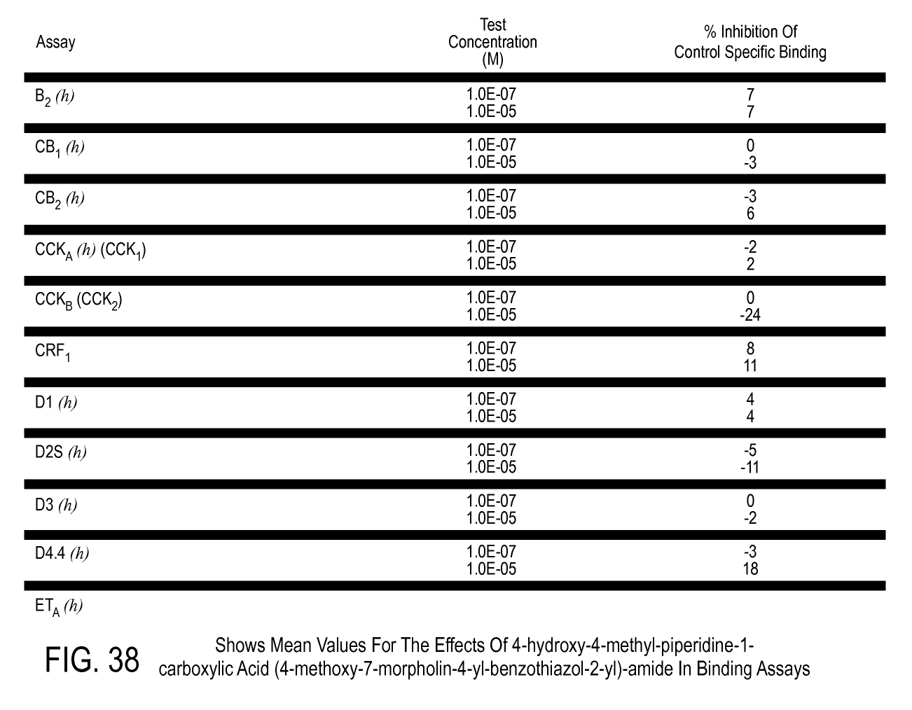

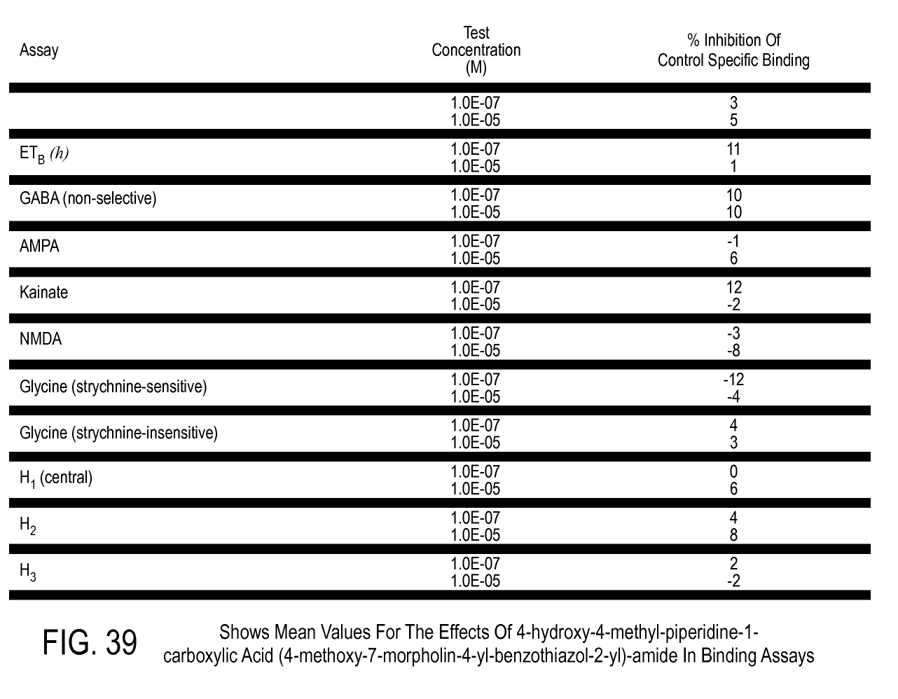

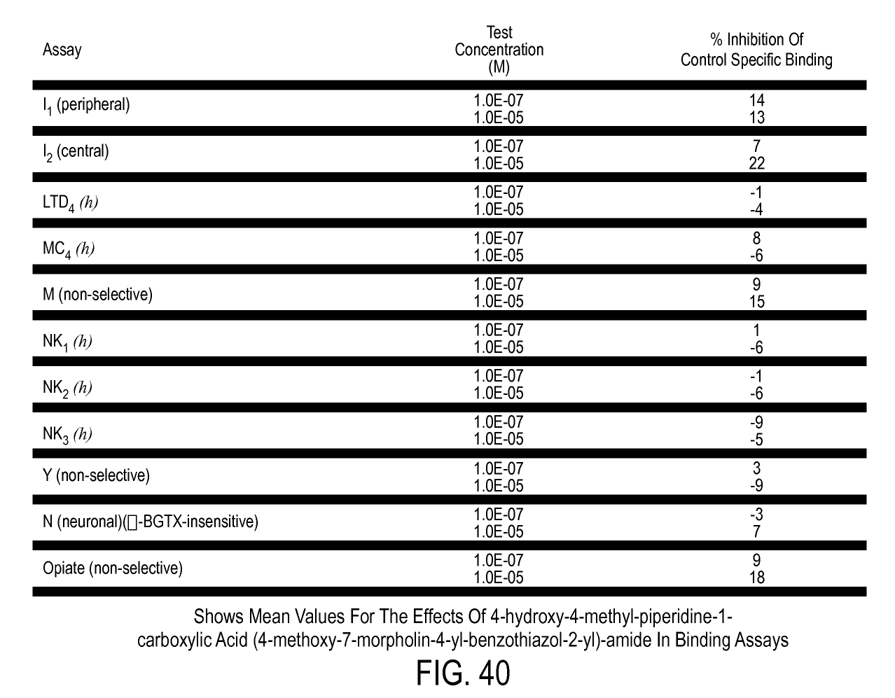

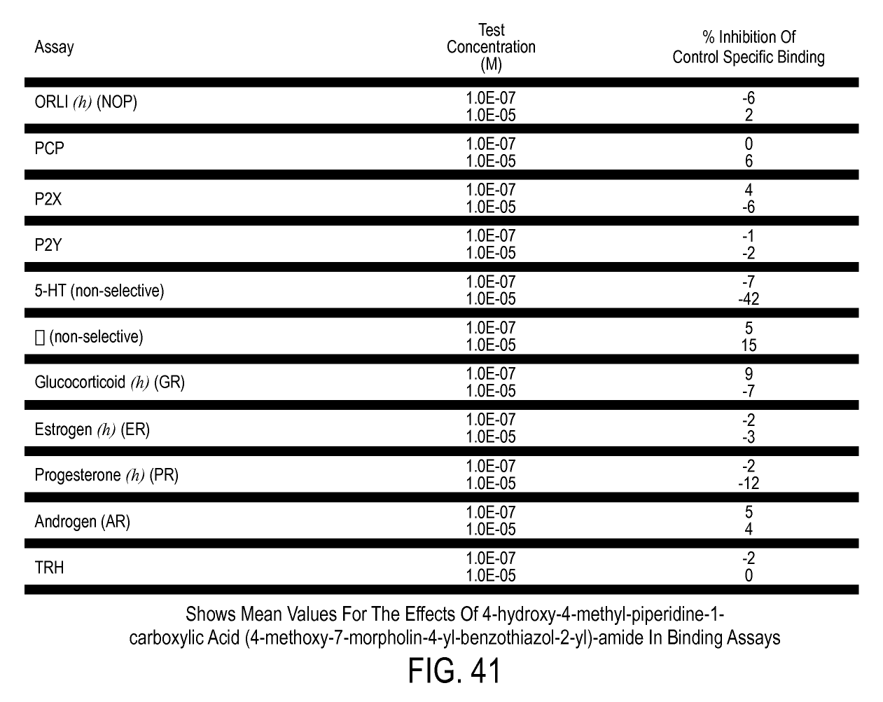

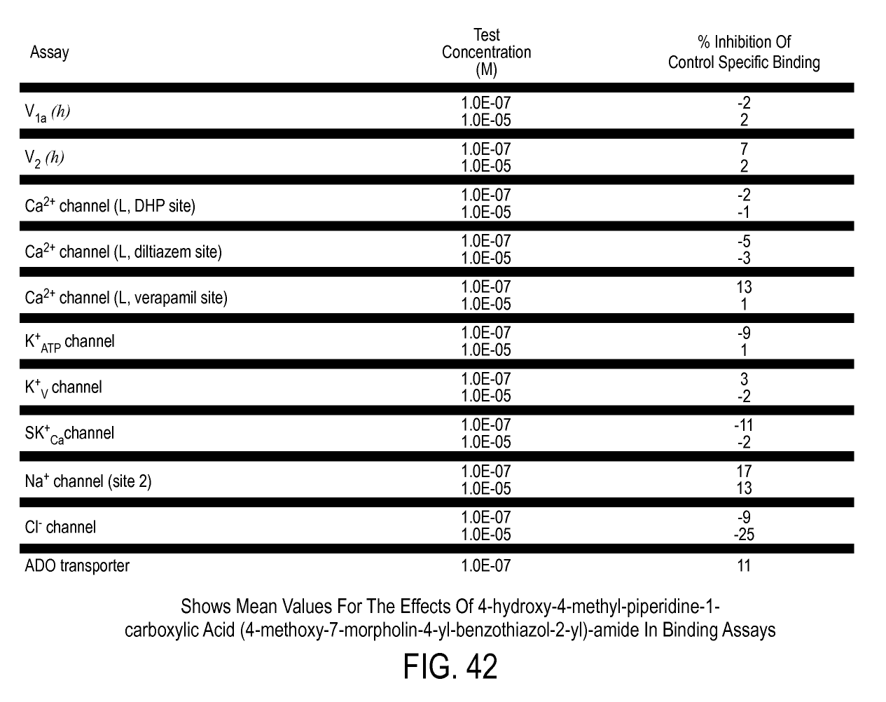

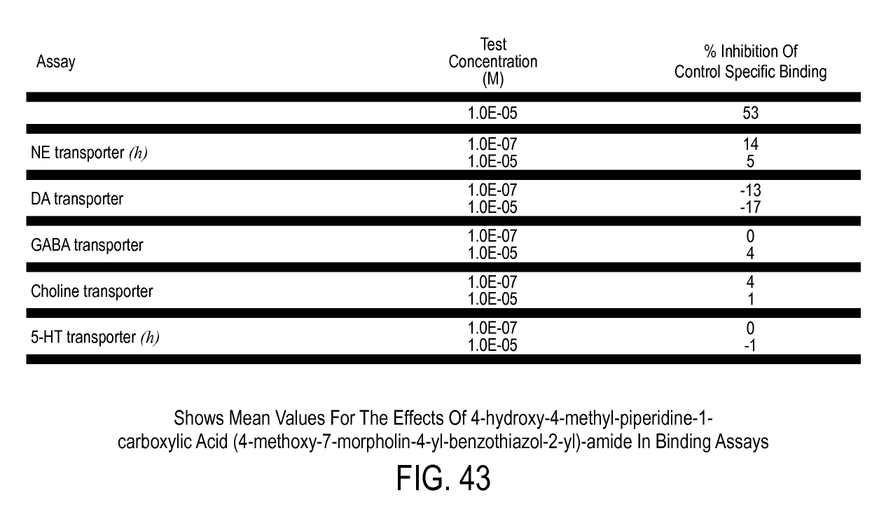

The purpose of this study was to investigate the effects of 4-hydroxy-4-methyl-piperidine-1-carboxylic acid (4-methoxy-7-morpholin-4-yl-benzothiazol-2-yl)-amide in various in vitro receptor binding and enzyme assays. See FIG. 22-25 for general procedures of binding assays. See FIG. 26-32 for experimental conditions of binding assays. See FIG. 37-43 for results.

The specific ligand binding to the receptors is defined as the difference between the total binding and the nonspecific binding determined in the presence of an excess of unlabelled ligand.

The results are expressed as a percent of control specific binding and as a percent inhibition of control specific binding obtained in the presence of 4-hydroxy-4-methyl-piperidine-1-carboxylic acid (4-methoxy-7-morpholin-4-yl-benzothiazol-2-yl)-amide. Individual and mean values are presented in the results section. The IC₅₀ values (concentration causing a half-maximal inhibition of control specific binding) and Hill coefficients (n_{H}) were determined by non-linear regression analysis of the competition curves using Hill equation curve fitting.

The inhibition constants (Kᵢ) were calculated from the Cheng Prusoff equation (Kᵢ=IC₅₀/(1+(L/K_{D})), where L=concentration of radioligand in the assay, and K_{D}=affinity of the radioligand for the receptor).

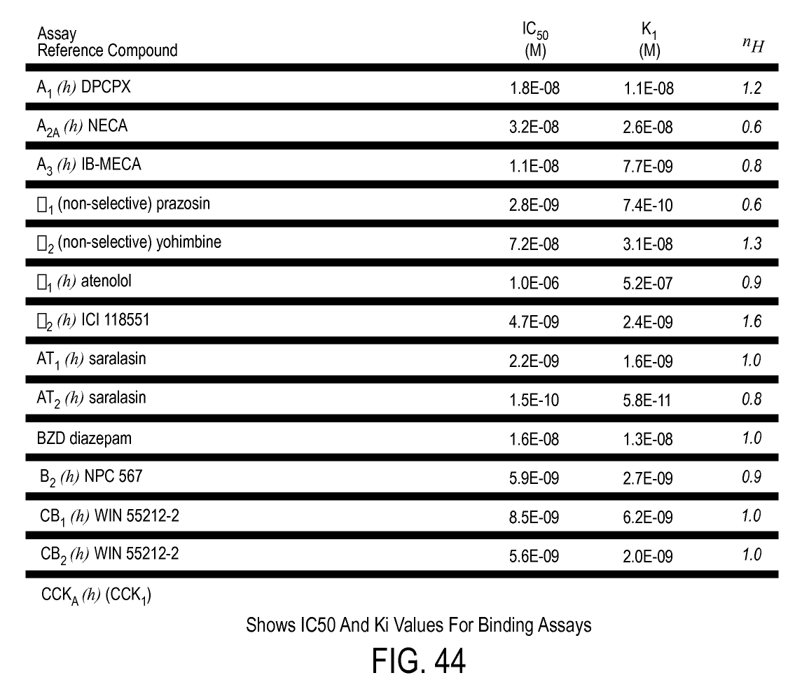

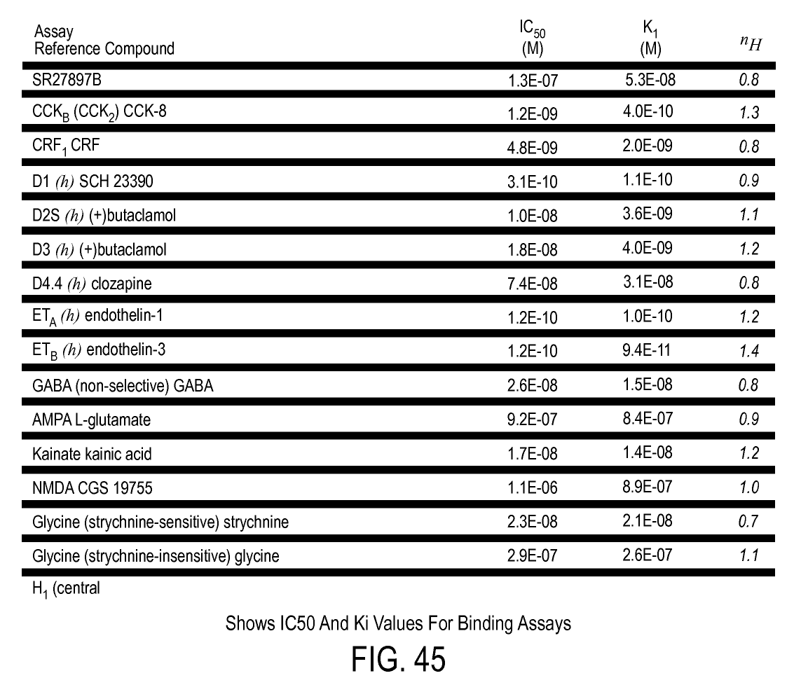

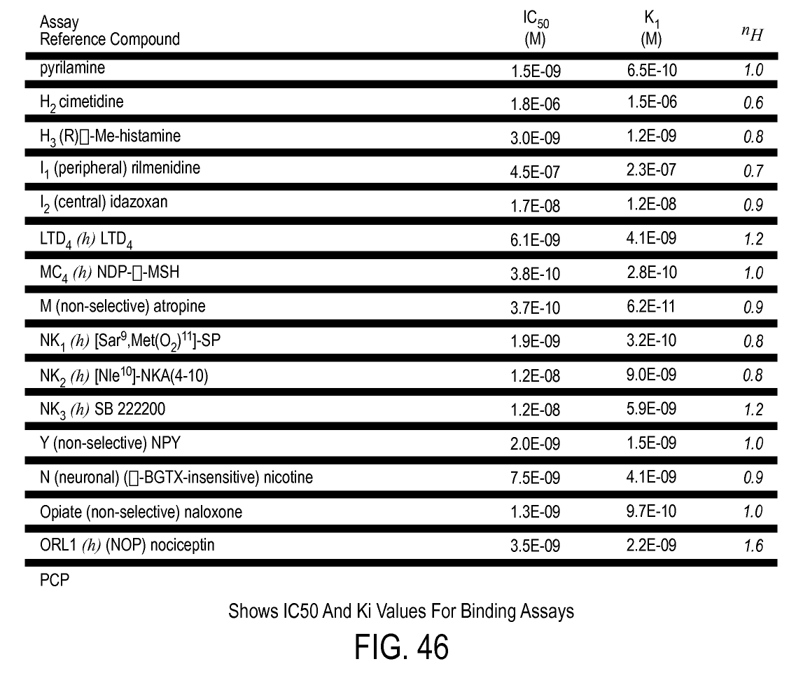

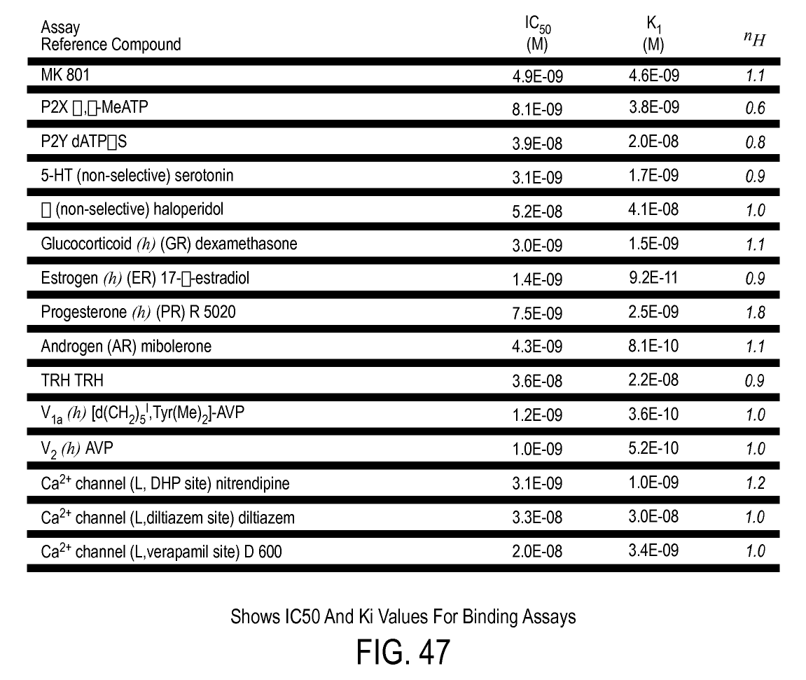

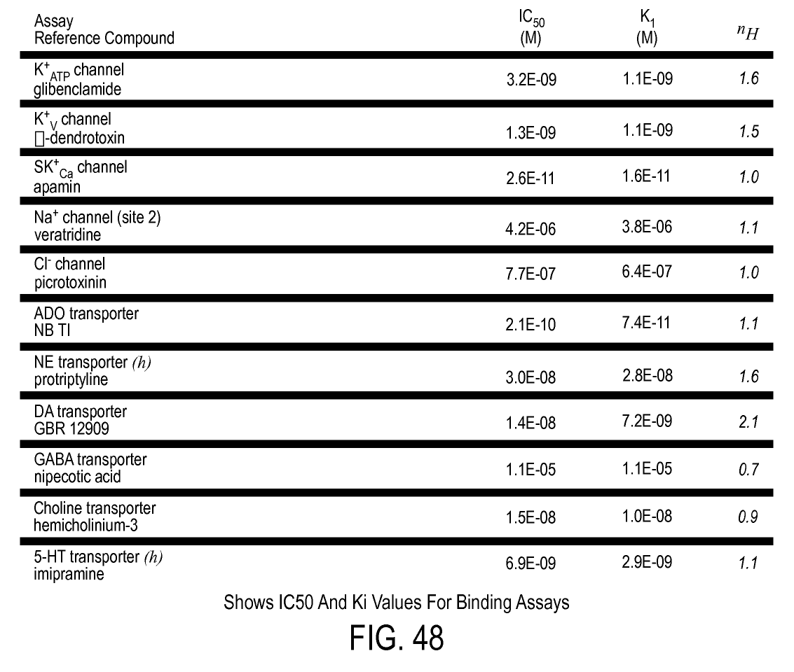

See FIG. 44-48 for IC50 and Ki values for binding assays.

For enzyme assays, the results are expressed as a percent of control values and as a percent variation of control values obtained in the presence of 4-hydroxy-4-methyl-piperidine-1-carboxylic acid (4-methoxy-7-morpholin-4-yl-benzothiazol-2-yl)-amide.

Individual and mean values are presented in the results section.

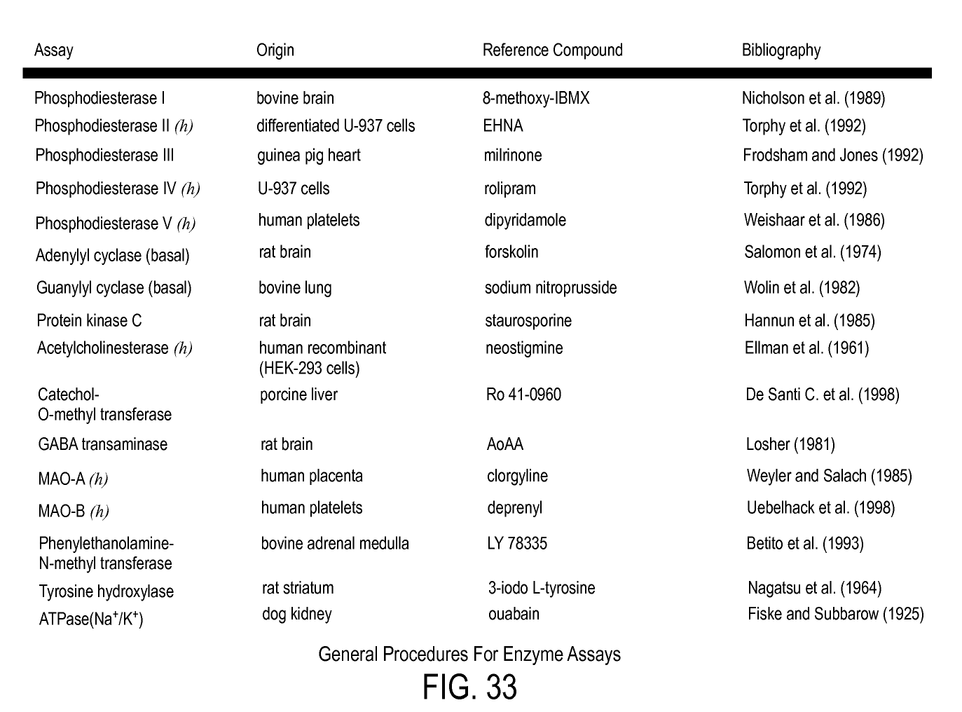

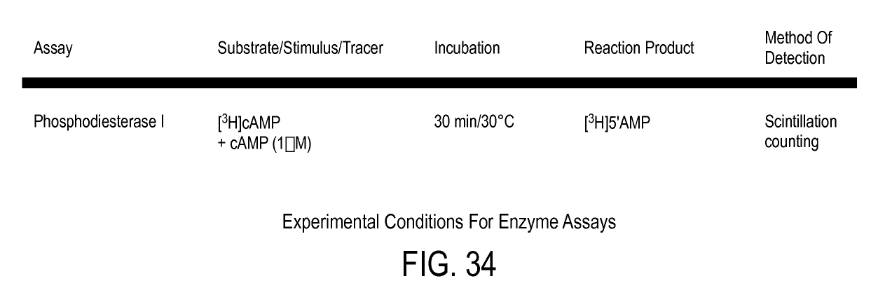

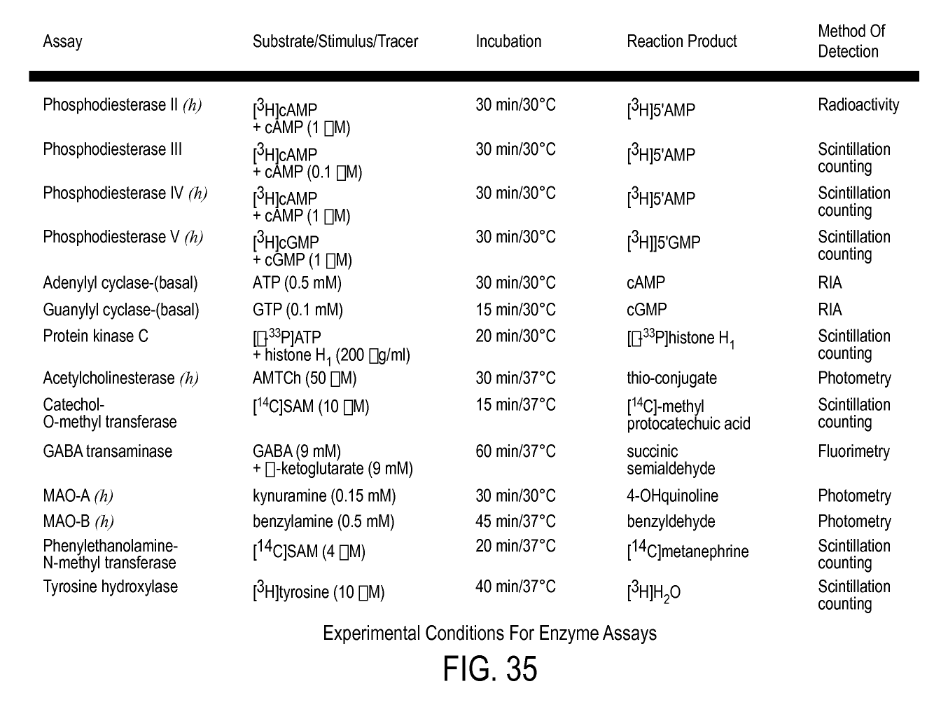

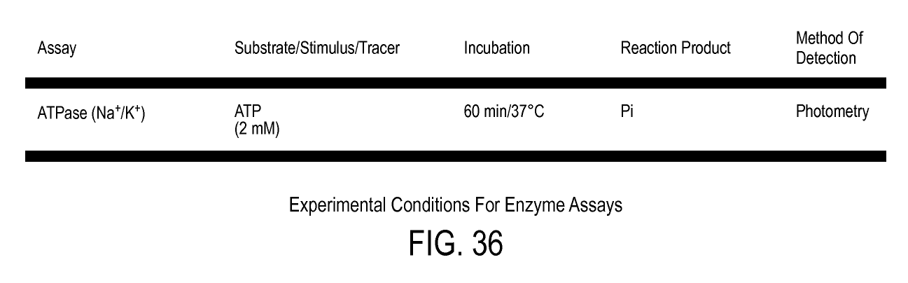

The IC₅₀ values (concentration causing a half-maximal inhibition of control values), EC₅₀ values (concentration causing a half-maximal stimulation of control values) and Hill coefficients (n_{H}) were determined by non-linear regression analysis of the concentration-response curves using Hill equation curve fitting. See FIG. 33 for general procedures and FIG. 34-36 for experimental conditions for enzyme assays.

In each experiment, the respective reference compound was tested concurrently with 4-hydroxy-4-methyl-piperidine-1-carboxylic acid (4-methoxy-7-morpholin-4-yl-benzothiazol-2-yl)-amide in order to assess the assay suitability. It was tested at several concentrations (for IC₅₀ or EC₅₀ value determination), and the data were compared with historical values determined at Cerep.

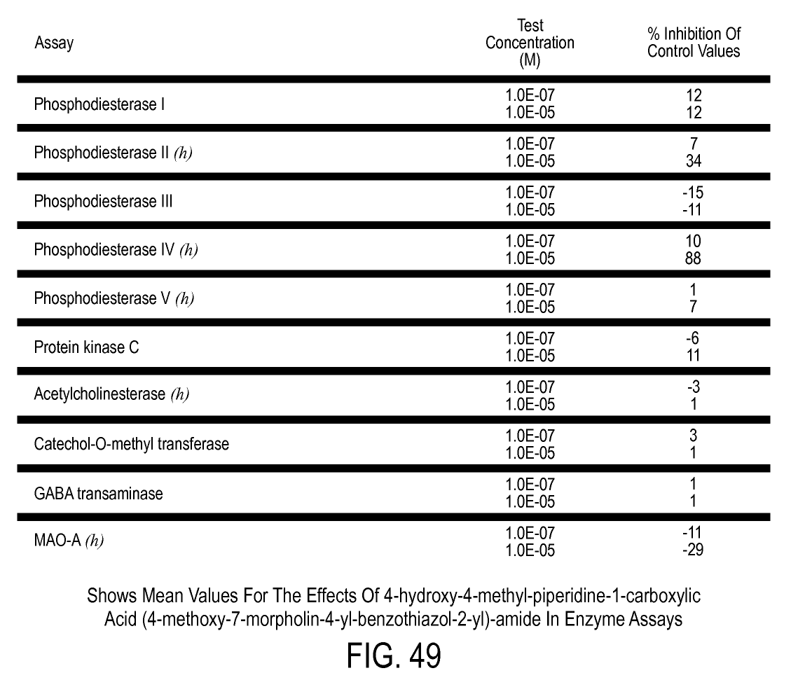

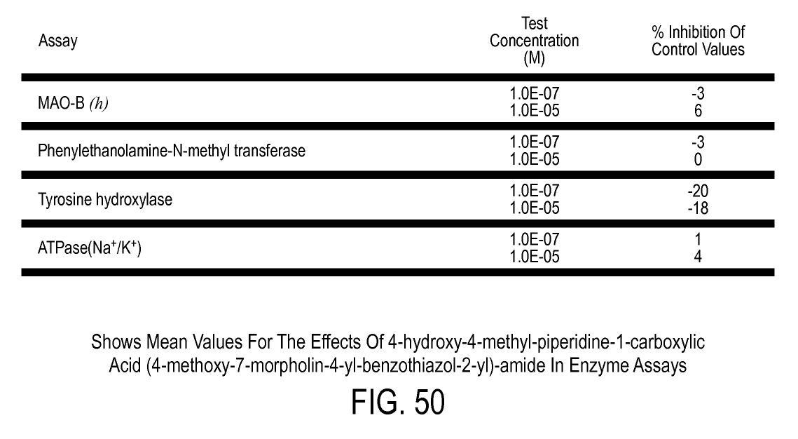

The mean values for the effects of 4-hydroxy-4-methyl-piperidine-1-carboxylic acid (4-methoxy-7-morpholin-4-yl-benzothiazol-2-yl)-amide are summarized in FIG. 49, 50, and 53.

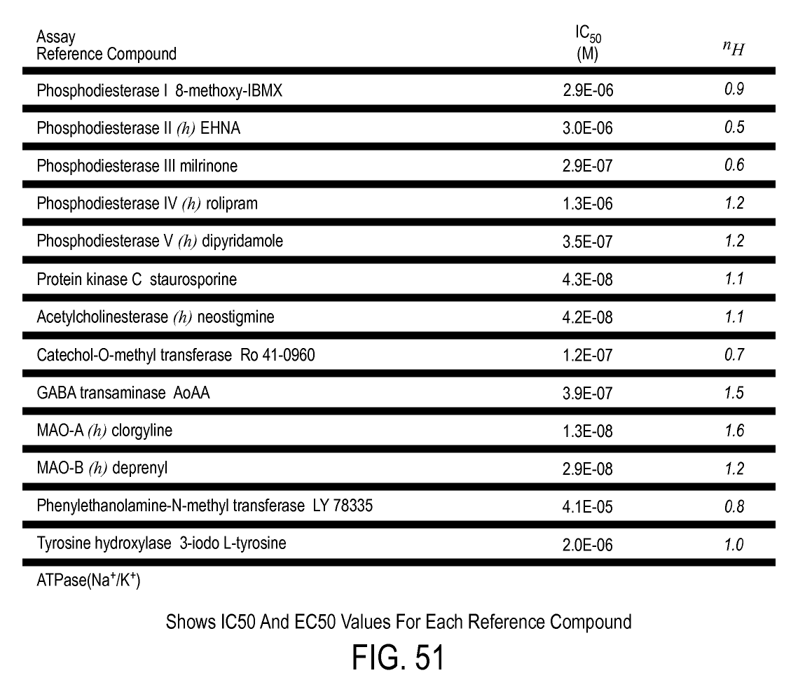

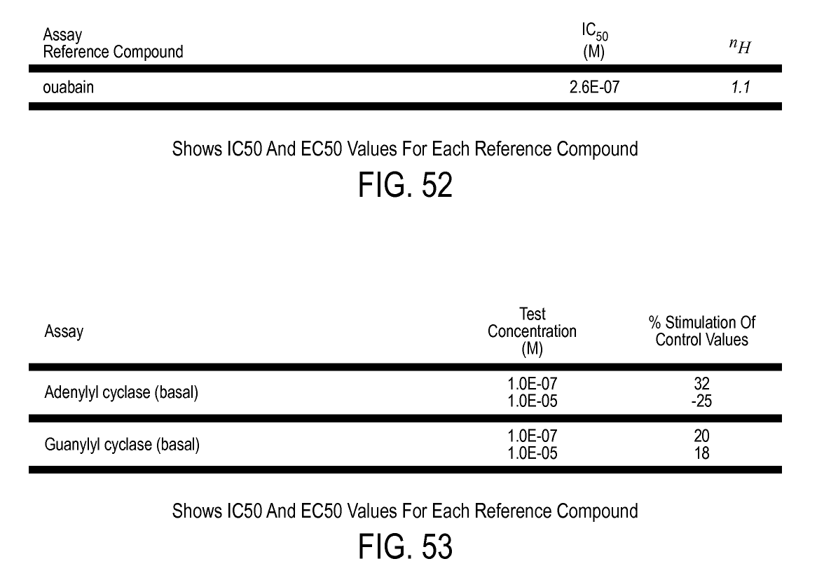

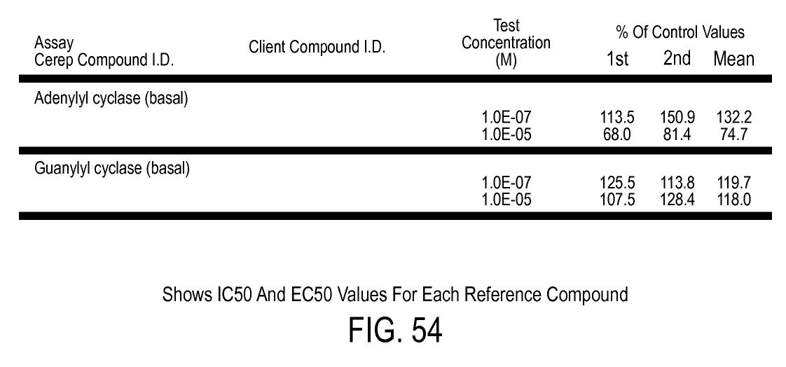

The IC₅₀ and Kᵢ values for each reference compound are indicated in FIGS. 51, 52, and 54. Each is within accepted limits of the historic average±0.5 log units.

### Example 7

The present study investigates the effect of 4-hydroxy-4-methyl-piperidine-1-carboxylic acid (4-methoxy-7-morpholin-4-yl-benzothiazol-2-yl)-amide in the APEC-induced hypolocomotion test. APEC (2-[(2-aminoethylamino)carbonylethyl-phenyl-ethylamino]-5′-ethylcarboxamidoadenosine) is an adenosine A_{2A} receptor agonist which reduces spontaneous motor activity in a dose-dependent manner. APEC-induced hypolocomotion is attenuated by selective A_{2A} receptor antagonists but not by selective A₁ receptor antagonists (Marston H M et al. Pharmacological characterization of a simple behavioral response mediated selectively by central adenosine A1 receptors, using in vivo and in vitro techniques. J Pharm Exp Ther. 1998; 285:1023-1030.)

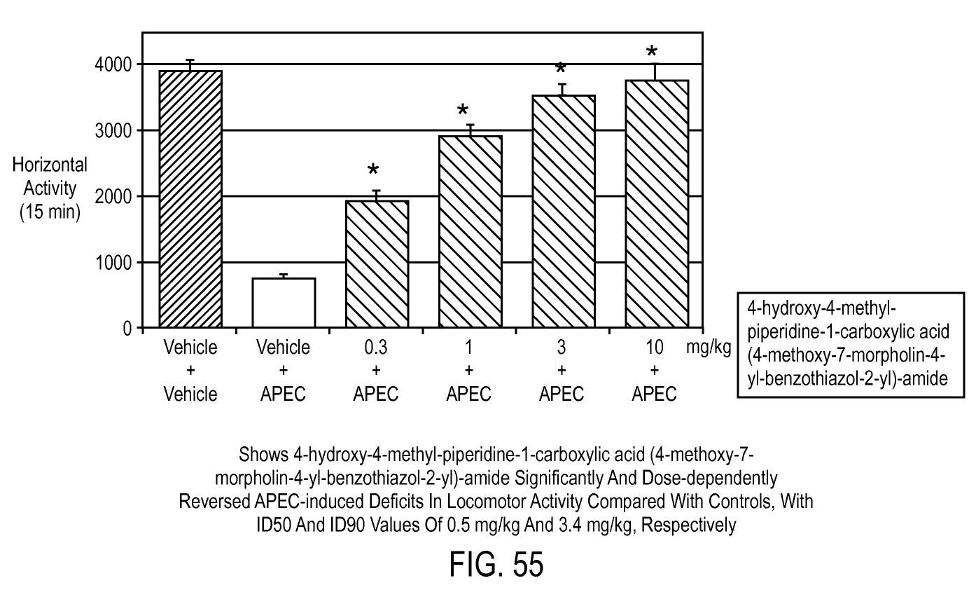

The ability of 4-hydroxy-4-methyl-piperidine-1-carboxylic acid (4-methoxy-7-morpholin-4-yl-benzothiazol-2-yl)-amide to block APEC-induced hypolocomotion in rats was assessed to determine its in vivo potency and its efficacy as a selective A_{2A} receptor antagonist. (FIG. 55)

Male adult Wistar rats (HanBrl: Wist (SPF) RCC) weighing approximately 140-200 g were used. The animals were housed in groups of four in Macrolon Type 3 cages (810 cm²) with sawdust bedding. Tap water and standard laboratory chow (Ratte Alleinfutter, extrudat No 3436; Provimikliba Kaiseraugst, Switzerland) were continuously available except during testing. The animal quarters were maintained on a 12:12 hr light-dark cycle with light onset at 6 a.m. Room temperature (21-23° C.) and humidity (55-65%) were kept constant. At the conclusion of testing, rats were euthanized by means of CO₂ inhalation. The experimental procedures used received prior approval from the City of Basel Cantonal Animal Protection Committee based on adherence to federal and local regulations on animal maintenance and testing. The methods were in compliance with ethical principles and guidelines for scientific experiments on animals recommended by the Swiss Academy of Medical Sciences and Swiss Academy of Sciences.

Locomotor activity was monitored with a Digiscan Animal Activity Monitoring system (Model RXYZCM Omnitech Electronics, Columbus, Ohio). The test boxes were made of Plexiglas (41×41×28 cm; W×L×H) and contained a thin layer of sawdust bedding. Each treatment group consisted of 16-24 rats. Two hours prior to locomotor activity recording, rats were treated with 4-hydroxy-4-methyl-piperidine-1-carboxylic acid (4-methoxy-7-morpholin-4-yl-benzothiazol-2-yl)-amide administered orally at dose 0.3, 1, 3, 10 mg/kg, followed 110 minutes later by a subcutaneous injection of 0.01 mg/kg of APEC. Control animals received vehicle only or vehicle and APEC. Ten minutes after APEC administration, animals were then placed in the test cages where horizontal activity was recorded for 15 min.

4-hydroxy-4-methyl-piperidine-1-carboxylic acid (4-methoxy-7-morpholin-4-yl-benzothiazol-2-yl)-amide was suspended in 0.3% Tween 80 in distilled water and APEC in 0.3% Tween 80 in saline 0.9%. 4-hydroxy-4-methyl-piperidine-1-carboxylic acid (4-methoxy-7-morpholin-4-yl-benzothiazol-2-yl)-amide-000-003 was administered p.o. (gavage) and APEC subcutaneously (s.c.). The injection volume was 5 ml/kg body weight. Doses refer to the free base of the drug.

Data was analyzed by Kruskal-Wallis ANOVA followed by Mann-Whitney U-test. A p-value lower than 0.05 was considered significant.

4-hydroxy-4-methyl-piperidine-1-carboxylic acid (4-methoxy-7-morpholin-4-yl-benzothiazol-2-yl)-amide significantly and dose-dependently reversed APEC-induced deficits in locomotor activity compared with controls, with ID₅₀ and ID₉₀ values of 0.5 mg/kg and 3.4 mg/kg, respectively.

In FIG. 55, data are mean±SEM based on 16-24 animals per group. * p<0.05 as determined by the Mann-Whitney test.

Oral administration of 4-hydroxy-4-methyl-piperidine-1-carboxylic acid (4-methoxy-7-morpholin-4-yl-benzothiazol-2-yl)-amide significantly reversed APEC-induced deficits in locomotor activity with ID50 and ID90 values of 0.5 mg/kg and 3.4 mg/kg respectively. These data provide in vivo evidence confirming that 4-hydroxy-4-methyl-piperidine-1-carboxylic acid (4-methoxy-7-morpholin-4-yl-benzothiazol-2-yl)-amide effectively inhibits brain adenosine A2a receptors following oral administration.

### Example 8

The present study investigates the antidepressant-like effects of 4-hydroxy-4-methyl-piperidine-1-carboxylic acid (4-methoxy-7-morpholin-4-yl-benzothiazol-2-yl)-amide in the swim stress test in rats. The swim stress test relies on the principle that when placed in water, rodents after an initial period of vigorous activity adopt a characteristic immobile posture, making only the minimal movements necessary to stay afloat. A reduction in the time of immobility is considered indicative of potential antidepressant-like properties of a particular drug (Porsolt R D et al. Behavioural despair in rats: a new model sensitive to antidepressant treatments. Eur. J. Pharmacol. 1978; 47:379-391).

Female adult Wistar rats (HanBrl: WIST (SPF); RCC Füllinsdorf) weighing approximately 100-130 g were used. The animals were housed in groups of four in Macrolon Type 3 cages (810 cm²) with sawdust bedding. Tap water and standard laboratory chow (Ratte Alleinfutter, extrudat No 3436; Provimikliba Kaiseraugst, Switzerland) were continuously available except during testing. The animal quarters were maintained on a 12:12 hr light-dark cycle with light onset at 6 a.m. Room temperature (21-23° C.) and humidity (55-65%) were kept constant. At the conclusion of testing, rats were euthanized by means of CO₂ inhalation. The experimental procedures used received prior approval from the City of Basel Cantonal Animal Protection Committee based on adherence to federal and local regulations on animal maintenance and testing. The methods were in compliance with ethical principles and guidelines for scientific experiments on animals recommended by the Swiss Academy of Medical Sciences and Swiss Academy of Sciences.

Naïve rats were individually forced to swim inside vertical plexiglass cylinders (height: 40 cm; diameter: 17.5 cm) containing 15 cm of water maintained at 23-24° C. After 15 min in the water, they were removed and allowed to dry for 15 min under a heating lamp before being returned to their home cage. They were replaced in the cylinders 24 h later and the total duration of immobility was measured during a 5-min test. The rat was judged to be immobile whenever it remained floating passively in the water in a slightly hunched but upright position, its head just above the surface 4-hydroxy-4-methyl-piperidine-1-carboxylic acid (4-methoxy-7-morpholin-4-yl-benzothiazol-2-yl)-amide was suspended in 0.3% Tween 80 in distilled water. 4-hydroxy-4-methyl-piperidine-1-carboxylic acid (4-methoxy-7-morpholin-4-yl-benzothiazol-2-yl)-amide was administered p.o. (gavage) 24 h, 16 h, and 2 h prior to the test. The injection volume was 5 ml/kg body weight. Doses refer to the free base of the drug.

Data were analyzed using an unpaired Student's t-test. A p-value lower than 0.05 was considered significant.

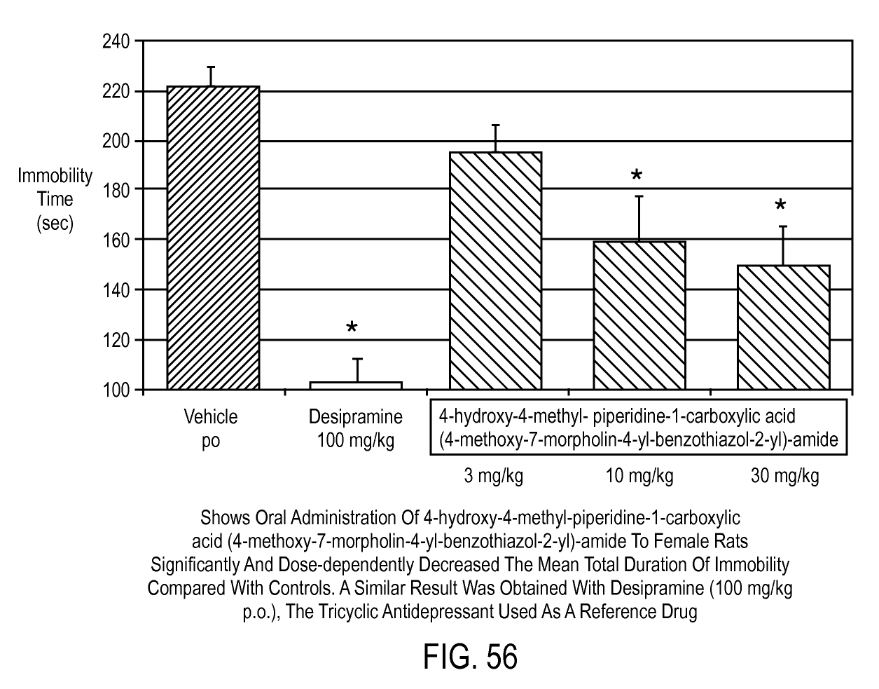

FIG. 56 shows that oral administration of 4-hydroxy-4-methyl-piperidine-1-carboxylic acid (4-methoxy-7-morpholin-4-yl-benzothiazol-2-yl)-amide to female rats significantly and dose-dependently decreased the mean total duration of immobility compared with controls. A similar result was obtained with desipramine (100 mg/kg p.o.), the tricyclic antidepressant used as a reference drug. Data are mean±SEM based on 8 animals per group. *p<0.05 based on Student's t-test. Oral administration of 4-hydroxy-4-methyl-piperidine-1-carboxylic acid (4-methoxy-7-morpholin-4-yl-benzothiazol-2-yl)-amide significantly and dose-dependently decreased the mean total duration of immobility in a swim stress test in rats. These data provide in vivo evidence for potential antidepressant-like properties of 4-hydroxy-4-methyl-piperidine-1-carboxylic acid (4-methoxy-7-morpholin-4-yl-benzothiazol-2-yl)-amide following oral administration

### Example 9

The present study investigates the potential antidepressant-like effects of 4-hydroxy-4-methyl-piperidine-1-carboxylic acid (4-methoxy-7-morpholin-4-yl-benzothiazol-2-yl)-amide in the chronic mild stress-induced anhedonia test in rats. The method follows that described by Moreau, J.-L., Bourson, A., Jenck, F., Martin, J. R. and Mortas, P. (1994) Curative effects of the atypical antidepressant mianserin in the chronic mild stress-induced anhedonia model of depression. J. Psychiatr. Neurosci. 19, 51-56 and Moreau, J.-L., Scherschlicht, R., Jenck, F. and Martin, J. R. (1995) Chronic mild stress-induced anhedonia model of depression: sleep abnormalities and curative effects of electroshock treatment. Behav. Pharmacol. 5, 682-687.

Adult male albino Wistar rats (HanBrl WIST (SPF), RCC Ltd, Füllinsdorf, Switzerland) weighing approximately 350 g at the start of the experiment were used. After surgery, rats were maintained individually in Macrolon type Ill containers (except when temporarily group housed as part of the stress regimen) under standard laboratory conditions (12 h light/dark cycle with light onset at 6:00 am, temperature of 21° C. to 23° C.) with free access to food (Kliba Miihlen, Kaiseraugst, Switzerland) and tap water. Stressed and control animals were housed in the same quarters, except as otherwise indicated. The experimental procedures used received prior approval from the City of Basel Cantonal Animal Protection Committee based on adherence to federal and local regulations on animal maintenance and testing. The methods were in compliance with ethical principles and guidelines for scientific experiments on animals recommended by the Swiss Academy of Medical Sciences and Swiss Academy of Sciences.

Animals were anesthetized with sodium ketamine hydrochloride 5% (90 mg/kg i.p.) and xylazine 2% (10 mg/kg i.p.) and administered buprenorphine (Temgesic, 0.03 mg/kg s.c.). Stainless-steel bipolar electrodes (MS 303/3, Plastics One Inc., Roanoke, Va., USA) were checked by passing a current through during immersion in a saline solution. If current leaks could be visualized by appearance of small bubbles along the electrode, it was discarded. Properly-insulated electrodes were stereotaxically implanted unilaterally in the mesolimbic system at the level of the ventral tegmental area of the midbrain (2 mm anterior from lambda, 0.3 mm lateral from the midline suture, and 8.5 mm ventral from the skull surface). Electrode tips were approximately 0.5 mm apart in the dorsoventral plane. Electrodes were implanted perpendicular to the horizontal plane, and the incisor bar adjusted to place lambda and bregma in the same horizontal plane. The electrode assembly was secured to the skull by four to five stainless-steel screws and an autopolymerizing resin. Animals were maintained post-operatively in a warm environment until fully awake and were given a SC injection of 0.03 mg/kg buprenorphine to minimize post-operative pain. They were allowed at least 5 days post-surgical recovery before starting training.

For the Ventral tegmentum self-stimulation (VTSS) procedure, the test chambers consisted of Plexiglas boxes (30×25˜25 cm) with a hole (2.5 cm in diameter) located in a sidewall 5 cm above the floor. The rat could interrupt a convergent light beam with a nose-poke to trigger electrical brain stimulation. Bipolar stimulation (0.5 s trains of monophasic square pulses of 0.1 ms duration) was delivered from a constant-current stimulator controlled by a PC computer which also recorded responding. In the training phase, each rat was placed into a test chamber and trained to make a nose-poke response for rewarding intracranial electrical stimulation. The frequency was kept at 70 Hz and the current intensity was made available which, for each individual rat, maintained the highest response rate without observable motor impairment. Training continued until stable responding was achieved. Then, the threshold for VTSS behavior was determined as described previously. Briefly, the frequency of stimulation was varied in a stepwise descending and ascending fashion, in steps of 10 Hz, until a criterion response rate was achieved (defined as 15 nose-poke responses per min). The stimulation intensity was maintained at the value previously found to produce the highest response rate in each individual rat. Animals were tested for 2 min at each frequency level and the number of nose-poke responses recorded. The mean response rate was calculated for each frequency level. In the absence of brain stimulation, response rate was usually lower than 10 nose-pokes per min (and never exceeded 15). The VTSS threshold was, therefore, defined as the mean of the ascending and descending frequencies eliciting 15 nose-pokes per min.

The stress regimen consisted each week of a variety of unpredictable, mild stressors such as repeated I-hr periods of confinement to small (24×10×9 cm) cages with bells ringing every 10 min, one period of continuous overnight illumination, one overnight period of food and water deprivation immediately followed by 2 hr of access to restricted food (scattering of 18 food pellets of 45 mg in the cage), one overnight period of water deprivation immediately followed by 1 hr exposure to an empty bottle, one overnight period of group housing in a damp cage (100 ml water in sawdust bedding). Animals were also maintained on a reversed light/dark cycle from Friday evening to Monday morning.

The experiments were started when the self-stimulation threshold of individual rats varied by less than 15% over three consecutive daily test sessions. Three groups of 7 to 8 rats each were subjected to the chronic mild stress regimen and two groups of 6 rats each were left undisturbed. From day 25 to day 46, two groups of stressed animals were dosed with 4-hydroxy-4-methyl-piperidine-1-carboxylic acid (4-methoxy-7-morpholin-4-yl-benzothiazol-2-yl)-amide (1 or 3 mg/kg i.p.) whereas the third group of stressed rats was injected with saline. During the same period of time, the two groups of nonstressed animals were treated with 4-hydroxy-4-methyl-piperidine-1-carboxylic acid (4-methoxy-7-morpholin-4-yl-benzothiazol-2-yl)-amide (3 mg/kg i.p.) or saline, respectively. ICSS thresholds were determined in the morning twice weekly and the threshold values obtained for each group on each test day were compared. Results are expressed as percentage change in ICSS threshold, representing the anhedonia index (the higher the increase in ICSS threshold, the greater the anhedonia).

Data were analyzed by a 2-factor repeated measures analysis of variance supplemented where appropriate by comparisons on individual days carried out with an unpaired t-test. A p-value of less than 0.05 was accepted as statistically significant.

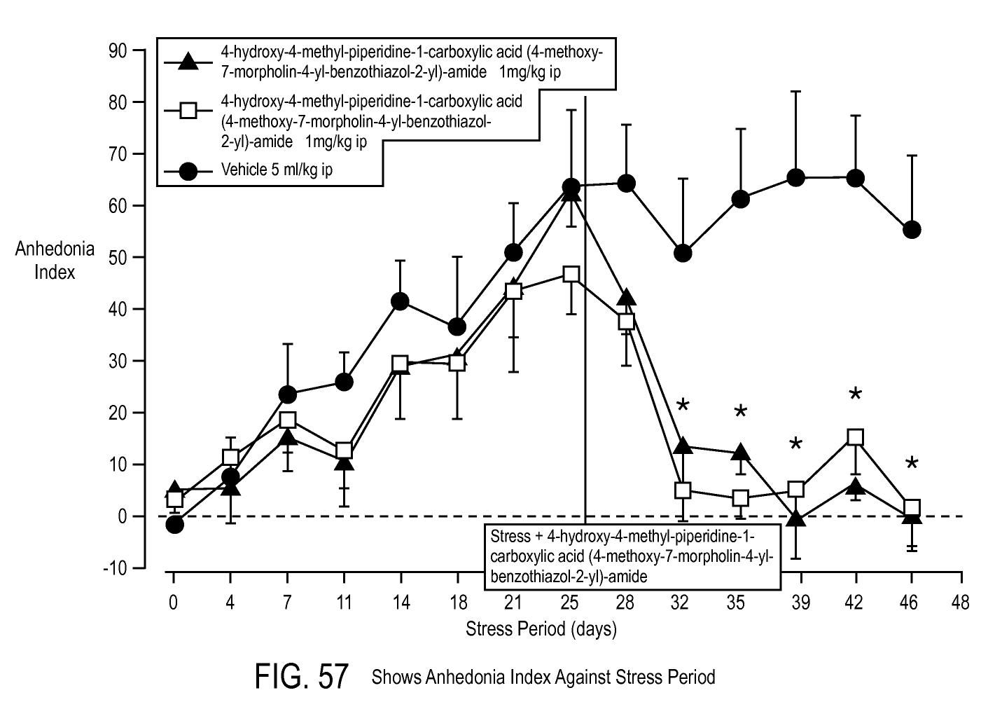

In all three stressed groups, there was a significant stress-induced increase in the anhedonia index (% change in ventral tegmentum self-stimulation threshold) [F(12,91)=8.89, ˜˜0.05; F(12,91)=12.54, ˜˜0.05, respectively]. When the stressed animals were treated with 1 or 3 mg/kg of 4-hydroxy-4-methyl-piperidine-1-carboxylic acid (4-methoxy-7-morpholin-4-yl-benzothiazol-2-yl)-amide from day 25 to day 46 of the stress period, the increased anhedonia index returned to baseline control levels (animals regained normal levels of reward sensitivity) after one to two weeks of treatment, while vehicle-treated stressed animals remained anhedonic. When comparing drug-treated and vehicle-treated groups, a clear curative anti-anhedonic effect of 4-hydroxy-4-methyl-piperidine-1-carboxylic acid (4-methoxy-7-morpholin-4-yl-benzothiazol-2-yl)-amide was found [F(12,156)=2.28, pcO.O5]. See FIG. 57. Data are mean±SEM based on 7-8 animals per group. *p<0.05 based on a 2-factor repeated measure ANOVA followed by an unpaired t-test.

These data show that when stressed anhedonic animals were curatively treated with 4-hydroxy-4-methyl-piperidine-1-carboxylic acid (4-methoxy-7-morpholin-4-yl-benzothiazol-2-yl)-amide (1, 3 mg/kg ip once a day), the stress-induced anhedonia was completely reversed. These results demonstrate potential antidepressant properties for 4-hydroxy-4-methyl-piperidine-1-carboxylic acid (4-methoxy-7-morpholin-4-yl-benzothiazol-2-yl)-amide.

### Example 10

The present study investigates the antidepressant/anxiolytic-like effects of 4-hydroxy-4-methyl-piperidine-1-carboxylic acid (4-methoxy-7-morpholin-4-yl-benzothiazol-2-yl)-amide in the differential-reinforcement-of-low-rate-30-seconds (DRL-30) test in rats. The method follows that described by Richards J B, Seiden L S. A quantitative interresponse-time analysis of DRL performance differentiates similar effects of the antidepressant desipramine and the novel anxiolytic gepirone. J Exp Anal Behav. 1991; 56:173-192. Stephens D N, Voet B. Differential effects of anxiolytic and non-anxiolytic benzodiazepine receptor ligands on performance of a differential reinforcement of low rate (DRL) schedule. Behav Pharmacol. 1994; 5:4-14. Typical antidepressants increase the inter-response times, therefore decreasing the rate of responding. They also increase the number of reinforcements. Opposite effects are observed with benzodiazepine-like anxiolytics or with atypical antidepressants.

Male adult Sprague-Dawley rats (Charles River, France) weighing approximately 350 g were used. The animals were housed one per cage in Macrolon Type 3 cages (810 cm²) with sawdust bedding and wood shavings. Animals had free access to tap water and restricted access (15 g per day) to standard laboratory chow (Ratte Alleinfutter, extrudat No 3436; Provimikliba Kaiseraugst, Switzerland). The animal quarters were maintained on a 12:12 hr light-dark cycle with light onset at 6 a.m. Room temperature (22±2° C.) and humidity (55-65%) were kept constant. The experimental procedures used received prior approval from the City of Basel Cantonal Animal Protection Committee based on adherence to federal and local regulations on animal maintenance and testing. The methods were in compliance with ethical principles and guidelines for scientific experiments on animals recommended by the Swiss Academy of Medical Sciences and Swiss Academy of Sciences.

The differential-reinforcement-of-low-rate-30-seconds (DRL-30) test was used. The apparatus consisted of a sound-attenuated standard Skinner box (28×21×21 cm) (MED Associates Inc.) fitted with a house light, one lever and a food pellet dispenser (45 mg food pellet). The lever was located on the left of the food receptacle connected to the pellet dispenser. The Skinner boxes were connected to a programming system (Kestrel Software, Conclusive Solutions, Harlow, UK) which controlled the experiment and collected the data automatically.

The rats were first submitted to lever-pressing acquisition sessions in the experimental chamber according to a fixed ratio (FR1) schedule of reinforcement. Reinforcements consisted of food pellets (45 mg Noyes Pellet “formula P”, NH, USA) delivered after each lever press. Subsequent to the lever-pressing acquisition stage, the animals were submitted to repeated training sessions according to a differential reinforcement of low rate (DRL) schedule. In this procedure, only responses occurring after a delay were rewarded (reinforced responses). Responses which occurred before the end of the delay were not reinforced and reset the delay for the following response. The delay was gradually increased from 5 seconds to 30 seconds (DRL-30) to achieve a stable level of DRL-30 performance at the end of the training stage before starting drug testing. Each training session lasted 15 minutes. The animals received p.o. administration of distilled water 2 hours before each session. In addition to the food pellet consumed in the Skinner box, each animal received a daily 15 g food ration in their home cages (at 5 p.m.)

Three measures were taken for each session: the total number of responses, the number of reinforcements (lever-presses occurring at least 30 seconds after the previous lever-press), and the mean inter-response times (average waiting time elapsed between successive lever-presses).

Drug testing was performed on animals having reached stable baseline DRL-30 performance over two consecutive weeks. Drug testing sessions were given twice weekly with at least two training session without drugs between two test sessions. Each animal was used as its own control and received all the selected treatments and controls in separate testing sessions. The sequence of treatments was determined by a randomization procedure to ensure even distribution of the different treatments in time. Each animal was always tested in the same Skinner box, in the same order and at the same time of the day. The test was performed blind. Testing was conducted in 11 animals. 4-hydroxy-4-methyl-piperidine-1-carboxylic acid (4-methoxy-7-morpholin-4-yl-benzothiazol-2-yl)-amide was suspended in 0.3% Tween 80 in distilled water. 4-hydroxy-4-methyl-piperidine-1-carboxylic acid (4-methoxy-7-morpholin-4-yl-benzothiazol-2-yl)-amide was evaluated at 3, 10 and 30 mg/kg administered p.o. (gavage) 2 hours prior to the test. The injection volume was 5 ml/kg body weight. Doses refer to the free base of the drug.

Data were analyzed using one-tailed paired t-test

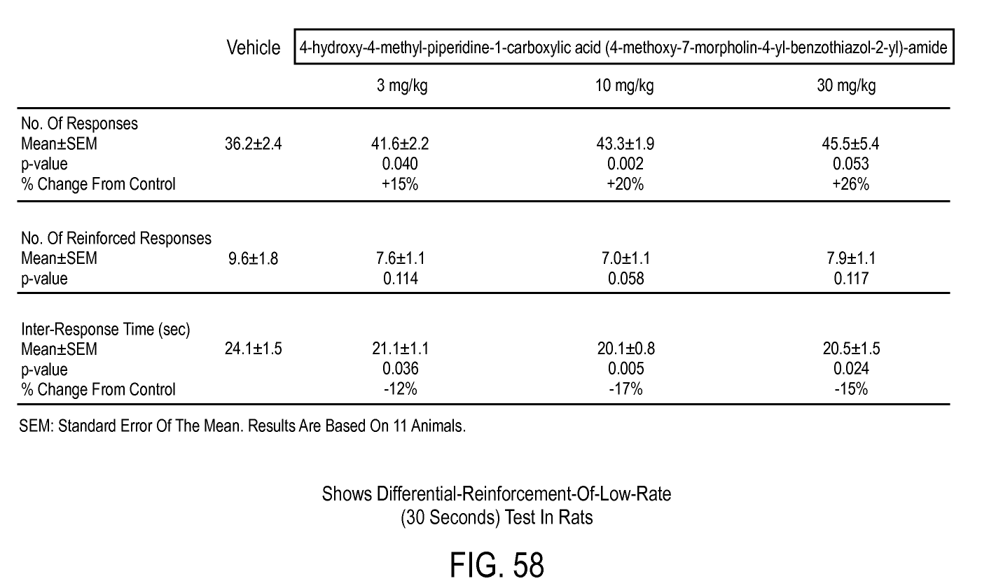

FIG. 58 shows that oral administration of 4-hydroxy-4-methyl-piperidine-1-carboxylic acid (4-methoxy-7-morpholin-4-yl-benzothiazol-2-yl)-amide (3, 10 and 30 mg/kg) to male rats significantly and dose-dependently increased the number of responses and decreased the mean inter-response times. It also tended to decrease the number of reinforcements. In rats previously trained to stable performance in the DRL-30 test, 4-hydroxy-4-methyl-piperidine-1-carboxylic acid (4-methoxy-7-morpholin-4-yl-benzothiazol-2-yl)-amide (3, 10, 30 mg/kg p.o.) significantly and dose-dependently increased the number of responses and decreased the mean inter-response times. It also tended to decrease the number of reinforcements. These data suggest either some anxiolytic-like activity or some atypical antidepressant-like properties.

### Example 11

The effectiveness of orally administered 4-hydroxy-4-methyl-piperidine-1-carboxylic acid (4-methoxy-7-morpholin-4-yl-benzothiazol-2-yl)-amide in reversing scopolamine-induced amnesia in a passive avoidance task was investigated in the present experiment.

Male adult Wistar rats (HanBrl: WIST (SPF); RCC Füllinsdorf) weighing approximately 94-113 g were used. The animals were housed in groups of four in Macrolon Type 3 cages (810 cm²) with sawdust bedding. Tap water and standard laboratory chow (Ratte Alleinfutter, extrudat No 3436; Provimikliba Kaiseraugst, Switzerland) were continuously available except during testing. The animal quarters were maintained on a 12:12 hr light-dark cycle with light onset at 6 a.m. Room temperature (21-23° C.) and humidity (55-65%) were kept constant. At the conclusion of testing, rats were euthanized by means of CO₂ inhalation. The experimental procedures used received prior approval from the City of Basel Cantonal Animal Protection Committee based on adherence to federal and local regulations on animal maintenance and testing. The methods were in compliance with ethical principles and guidelines for scientific experiments on animals recommended by the Swiss Academy of Medical Sciences and Swiss Academy of Sciences.

Passive avoidance training was carried out in a test chamber (40 cm×31 cm×29 cm) which included a grid floor composed of stainless-steel rods through which a constant current, scrambled electric shock (1.1 mA) could be continuously delivered. A 0.5 cm thick plastic platform (15 cm×15 cm) covered the grid in one corner. Each training session began with the placement of a rat on the platform with the adjacent grid electrified. When the rat stepped off the platform onto the grid it automatically received a foot shock and typically moved rapidly back onto the platform. The rat was then nudged gently onto the grid several times until this was actively resisted, thus, indicating that the rat had learned the association between stepping onto the grid floor and receiving a foot shock. Any animals exhibiting clear signs of marked drug-induced motor impairment/sedation were not tested at all. A retention test was done in the same apparatus with each rat placed on the plastic platform with the adjacent platform (which was not electrified). Stepping down onto the platform within the initial 60 sec of the retention trial or failing to subsequently resist being gently pushed onto the grid floor was judged as a failure to exhibit avoidance (and, thus, evidence for amnesia). Testing was carried out between 7 a.m. and 3 p.m. The observer was blind with respect to the treatment conditions when retention evaluation was done.

Immediately following passive avoidance acquisition, rats were alternatively assigned to one of the following treatment conditions, which were administered concomitantly:

1) Vehicle (sc)+Vehicle (po)

2) 1 mg/kg scopolamine HBr (sc)+Vehicle (po)

3) 1 mg/kg scopolamine HBr (sc)+4-hydroxy-4-methyl-piperidine-1-carboxylic acid (4-methoxy-7-morpholin-4-yl-benzothiazol-2-yl)-amide at doses 3, 10, 30 or 100 mg/kg, (po)

Two hours after the training and the post-training treatment, each rat was evaluated for retention of the passive avoidance response in the retention test. The data collected for rats, which received immediately post-training treatment with vehicle (sc)+vehicle (po) provides a measure of the baseline level of retention in the absence of scopolamine amnesia (not included in the statistical analysis). N=16 per treatment group.

4-hydroxy-4-methyl-piperidine-1-carboxylic acid (4-methoxy-7-morpholin-4-yl-benzothiazol-2-yl)-amide was prepared immediately prior to use in a vehicle of 0.3% (v/v) Tween-80 in NaCl (0.9%) and ultrasonified (Model Digital S, Transsonic). The injection volume was 5 ml/kg body weight. Doses refer to the free base of the drug. Scopolamine hydrobromide was prepared freshly in 0.3% (v/v) Tween-80-NaCl (0.9%) prior to use (injection volume 2 mg/kg, s.c.).

Statistical evaluation was done on these (all-or-none) data with a two-tailed Chi-square test (Statview for windows 92/98; version 5.0.1) to compare the proportion of scopolamine-injected rats exhibiting a retention impairment under each of the drug conditions to that observed after vehicle treatment. A p-value of 0.05 or less was accepted as statistically significant.

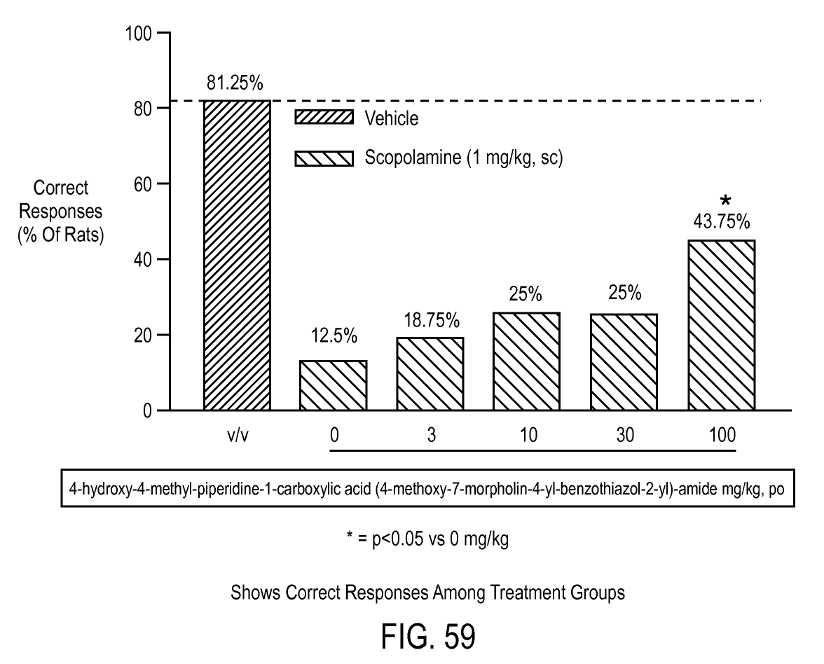

FIG. 59 shows that a significantly greater proportion of rats trained in the passive avoidance task treated post-training with vehicle (sc)+vehicle (po) exhibited retention 2 hours later than rats which received scopolamine (sc)+vehicle (Po), 81.25% versus 12.5% respectively. A significantly greater proportion (43.75%; P<0.05) of scopolamine-treated animals which also received oral administration of 100 mg/kg 4-hydroxy-4-methyl-piperidine-1-carboxylic acid (4-methoxy-7-morpholin-4-yl-benzothiazol-2-yl)-amide exhibited passive avoidance retention as compared to the scopolamine-treated group which also received oral vehicle.

Data are Correct Responses (% of animals; N=16 per group). *p<0.05 based on Chi-square test (two-tailed).

### Example 12

Adult male Sprague Dawley rats weighing approximately 200 g at the start of the experiment were used. They were housed in groups of four in Macrolon Type 3 cages (810 cm²) with sawdust bedding. The animals were maintained on a 12:12 h light-dark cycle with light onset at 6 am., with free access to tap water and food (Promikliba Kaiseraugst, Switzerland). Room temperature (21-23° C.) and humidity (55-65%) were kept constant.

The elevated plus-maze consisted of two open arms perpendicular to two closed arms (each arm was 10 cm wide×50 cm long) extending from a small open central area. The apparatus was constructed from grey polyvinylchloride plastic and placed 50 cm above the floor. The closed arms, opposite to one other, had a surrounding wall of 48 cm height. The apparatus was situated in a sound-attenuated observation room with controlled illumination (200 lux on the central platform of the plus-maze). Rats were tested in a randomized order. The test started by placing the animal on the central platform facing a closed arm. The duration of the test was 5 minutes. 70% ethanol was used to clean the apparatus prior to the introduction of each animal.

The plus-maze was positioned in the middle of a closed black environment with the animal observed via a closed circuit video camera mounted above the maze. Behavioral analysis was conducted using a computerized system (Ethovision, Noldus Information Technology, The Netherlands).

Each treatment group consisted of 9 to 12 rats. Each animal was used only for a single experiment. Rats were treated with either 4-hydroxy-4-methyl-piperidine-1-carboxylic acid (4-methoxy-7-morpholin-4-yl-benzothiazol-2-yl)-amide at doses 3, 10 and 30 mg/kg, vehicle (0.3% Tween 80/0.9% NaCl) or Chlordiazepoxide 10 mg/kg, as a positive control. After drug administration, rats were isolated in small cages without sawdust and water. After one hour, they were placed in the plus-maze.

The measures selected to represent anxiolytic-like behavior was the time spent (sec) within the open arms, the distance traveled (cm) within the open arms, and the number of transitions between the central platform and the open arms. The measure used to quantify motor activity was the distance traveled per second (speed) within the closed arms.

4-hydroxy-4-methyl-piperidine-1-carboxylic acid (4-methoxy-7-morpholin-4-yl-benzothiazol-2-yl)-amide and chlordiazepoxide was suspended in distilled water containing 0.3% Tween 80/0.9% NaCl. The compounds were administered p.o. (gavage) in a volume of 5 ml/kg body weight. Control animals received equal volume injections of vehicle.

For 4-hydroxy-4-methyl-piperidine-1-carboxylic acid (4-methoxy-7-morpholin-4-yl-benzothiazol-2-yl)-amide, statistical analysis was performed using one-way analysis of variance (ANOVA), followed by a post-hoc Dunnett's test. A p-value lower than 0.05 was considered significant. The effects of chlordiazepoxide were analysed using a t-test.

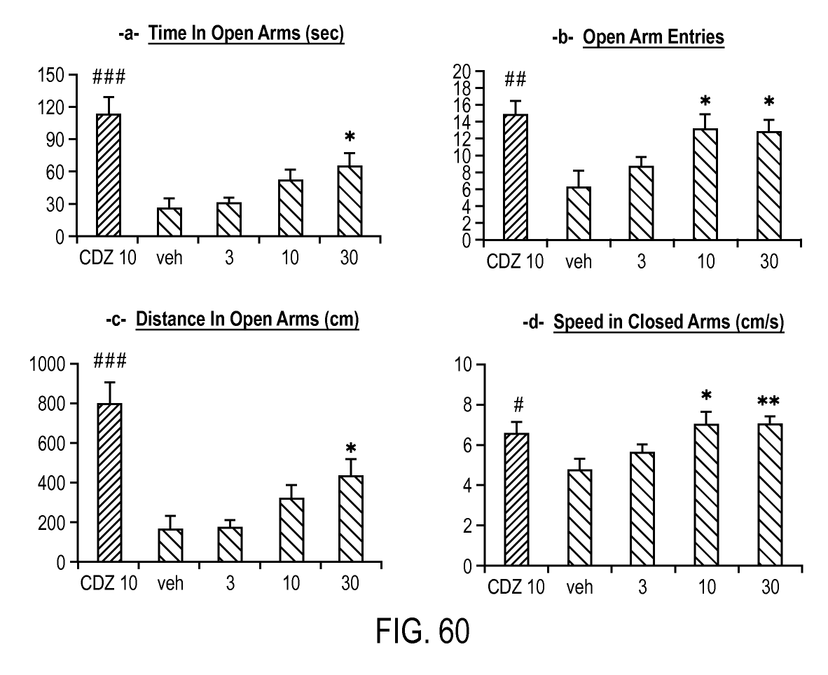

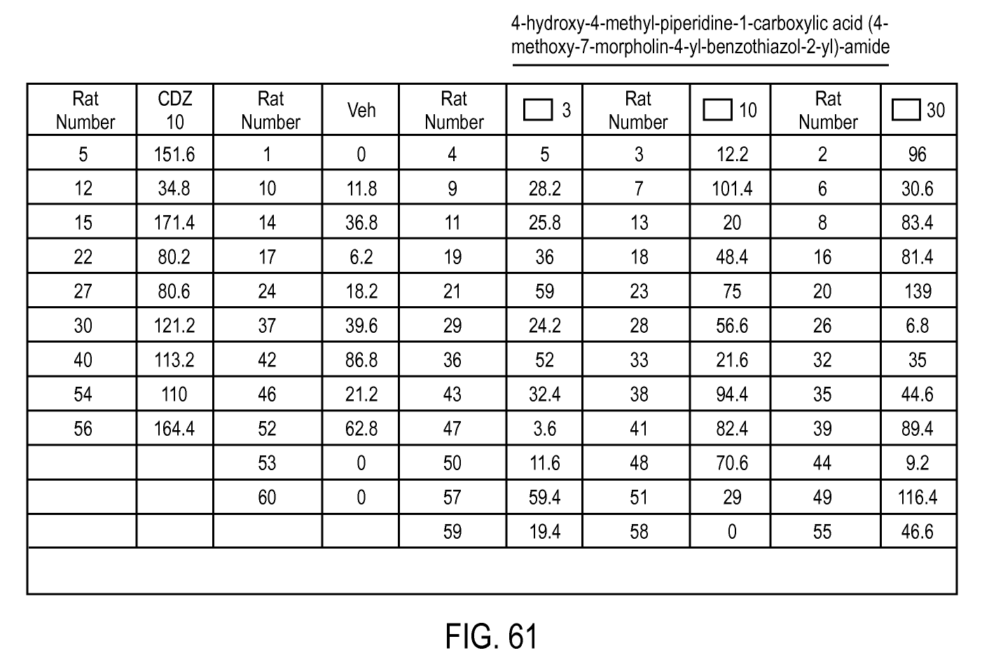

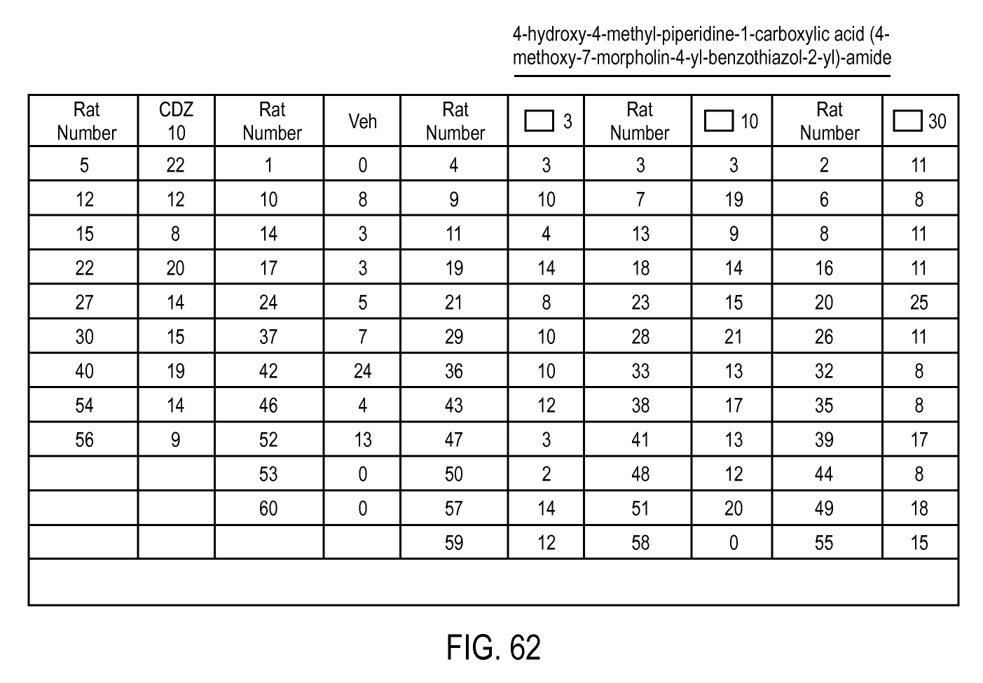

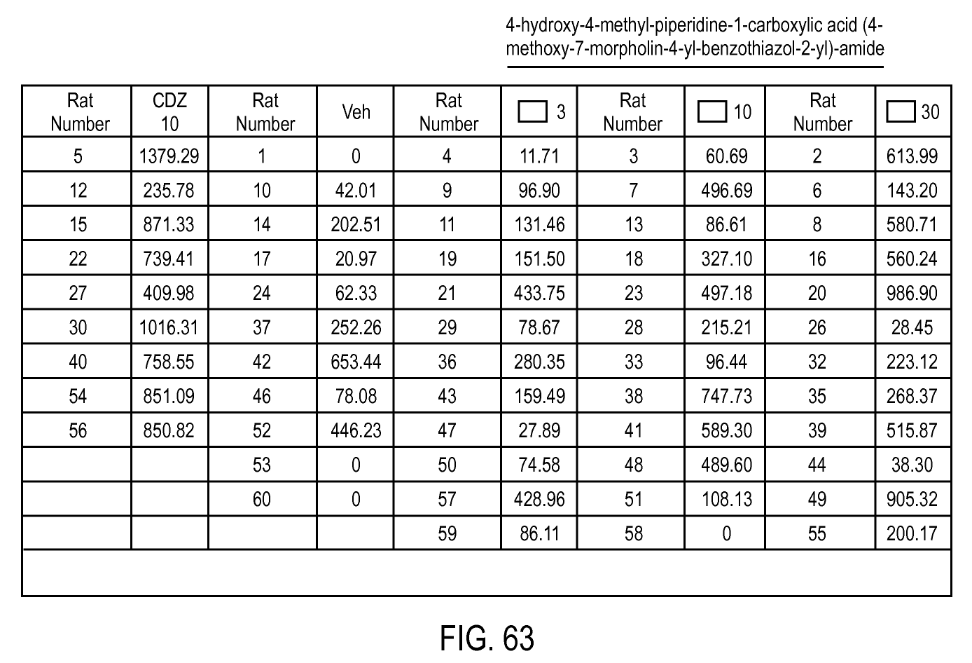

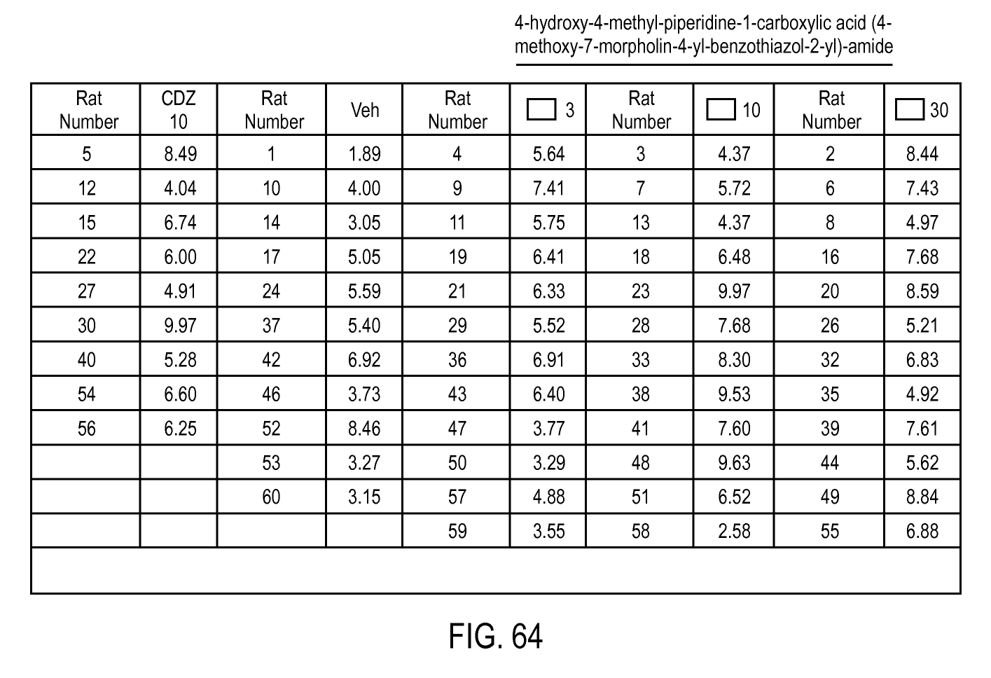

4-hydroxy-4-methyl-piperidine-1-carboxylic acid (4-methoxy-7-morpholin-4-yl-benzothiazol-2-yl)-amide produced a dose-dependent increase in time spent, distance traveled, and in the number of entries into these open arms (FIGS. 60-64). The lowest dose that reached statistical significance was 10 mg/kg (open arm entries) and 30 mg/kg (time spent, and distance traveled in the open arms). 4-hydroxy-4-methyl-piperidine-1-carboxylic acid (4-methoxy-7-morpholin-4-yl-benzothiazol-2-yl)-amide also increased speed in the closed arms reaching statistical significance at 10 and 30 mg/kg, similar to chlordiazepoxide. The maximal effect was observed at 30 mg/kg for all parameters measured.

In the Elevated Plus-Maze, 4-hydroxy-4-methyl-piperidine-1-carboxylic acid (4-methoxy-7-morpholin-4-yl-benzothiazol-2-yl)-amide significantly and dose-dependently increased the time spent, distance traveled, and the number of entries into the open arms. These results provide in vivo evidence for anxiolytic-like properties of 4-hydroxy-4-methyl-piperidine-1-carboxylic acid (4-methoxy-7-morpholin-4-yl-benzothiazol-2-yl)-amide.

It will be readily apparent to one of ordinary skill in the relevant arts that other suitable modifications and adaptations to the methods and applications described herein are suitable and may be made without departing from the scope of the invention or any embodiment thereof. While the invention has been described in connection with certain embodiments, it is not intended to limit the invention to the particular forms set forth, but on the contrary, it is intended to cover such alternatives, modifications and equivalents as may be included within the spirit and scope of the invention as defined by the following claims.

1. A method of treating a patient diagnosed with post-traumatic stress disorder, comprising administering to the patient a therapeutically effective amount of 4-hydroxy-4-methyl-piperidine-1-carboxylic acid (4-methoxy-7-morpholin-4-yl-benzothiazol-2-yl)-amide.

2. The method of claim 1, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

3. The method of claim 2, wherein the at least one other agent is a SSRI selected from paroxetine, sertraline, citalopram, escitalopram, and fluoxetine.

4. The method of claim 2, wherein the at least one other agent is a SNRI selected from duloxetine, mirtazapine, and venlafaxine.

5. The method of claim 2, wherein the at least one other agent is a NRI selected from bupropion and atomoxetine.

6. The method of claim 2, wherein the at least one other agent is a dopamine β-hydroxylase inhibitor selected from nepicastat and disulfiram.

7. The method of claim 1, wherein the 4-hydroxy-4-methyl-piperidine-1-carboxylic acid (4-methoxy-7-morpholin-4-yl-benzothiazol-2-yl)-amide reduces at least one A_{2A} receptor activity in the patient.

8. The method of claim 1, wherein the 4-hydroxy-4-methyl-piperidine-1-carboxylic acid (4-methoxy-7-morpholin-4-yl-benzothiazol-2-yl)-amide modulates dopaminergic signaling in the patient.

9. The method of claim 1, wherein the 4-hydroxy-4-methyl-piperidine-1-carboxylic acid (4-methoxy-7-morpholin-4-yl-benzothiazol-2-yl)-amide reduces at least one of the frequency and intensity of at least one sign of the post-traumatic stress disorder in the patient.

10. The method of claim 1, wherein the 4-hydroxy-4-methyl-piperidine-1-carboxylic acid (4-methoxy-7-morpholin-4-yl-benzothiazol-2-yl)-amide reduces at least one of the frequency and intensity of at least one symptom post-traumatic stress disorder in the patient.

11. The method of claim 1, wherein the 4-hydroxy-4-methyl-piperidine-1-carboxylic acid (4-methoxy-7-morpholin-4-yl-benzothiazol-2-yl)-amide reduces at least one of the frequency and intensity of at least one symptom cluster of the post-traumatic stress disorder in the patient, wherein the symptom cluster is selected from re-experiencing/intrusion, avoidance/numbing, and hyperarousal.

12. The method of claim 1, wherein the patient is a child or an adolescent.

13. The method of claim 12, wherein the 4-hydroxy-4-methyl-piperidine-1-carboxylic acid (4-methoxy-7-morpholin-4-yl-benzothiazol-2-yl)-amide reduces at least one of the frequency and intensity of at least one sign or symptom of the post-traumatic stress disorder in the patient, wherein the sign or symptom is selected from disorganized or agitated behavior, repetitive play that expresses aspects of the trauma, frightening dreams which lack recognizable content, and trauma-specific reenactment.

14. The method of claim 1, wherein the 4-hydroxy-4-methyl-piperidine-1-carboxylic acid (4-methoxy-7-morpholin-4-yl-benzothiazol-2-yl)-amide reduces the incidence of at least one disorder comorbid with post-traumatic stress disorder selected from drug abuse, alcohol abuse, and depression in the patient.

15. A method of treating post-traumatic stress disorder in a patient comprising:
diagnosing the patient with post-traumatic stress disorder;
administering to the patient a therapeutically effective amount of 4-hydroxy-4-methyl-piperidine-1-carboxylic acid (4-methoxy-7-morpholin-4-yl-benzothiazol-2-yl)-amide;
assessing at least one of sign, symptom, and symptom cluster of post-traumatic stress disorder; and
determining that the post-traumatic stress syndrome is improved if the 4-hydroxy-4-methyl-piperidine-1-carboxylic acid (4-methoxy-7-morpholin-4-yl-benzothiazol-2-yl)-amide reduces at least one of sign, symptom, and symptom cluster of post-traumatic stress disorder.

16. The method of claim 15, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

17. The method of claim 15, wherein the 4-hydroxy-4-methyl-piperidine-1-carboxylic acid (4-methoxy-7-morpholin-4-yl-benzothiazol-2-yl)-amide reduces at least one of the frequency and intensity of at least one sign of the post-traumatic stress disorder in the patient.

18. The method of claim 15, wherein the 4-hydroxy-4-methyl-piperidine-1-carboxylic acid (4-methoxy-7-morpholin-4-yl-benzothiazol-2-yl)-amide reduces at least one of the frequency and intensity of at least one symptom post-traumatic stress disorder in the patient.

19. The method of claim 15, wherein the 4-hydroxy-4-methyl-piperidine-1-carboxylic acid (4-methoxy-7-morpholin-4-yl-benzothiazol-2-yl)-amide reduces at least one of the frequency and intensity of at least one symptom cluster of the post-traumatic stress disorder in the patient, wherein the symptom cluster is selected from re-experiencing/intrusion, avoidance/numbing, and hyperarousal.

20. A method of improving resilience in a patient comprising administering a therapeutically effective amount of 4-hydroxy-4-methyl-piperidine-1-carboxylic acid (4-methoxy-7-morpholin-4-yl-benzothiazol-2-yl)-amide.

21. The method of claim 20, wherein the method further comprises coadministering a therapeutically effective amount of at least one other agent, selected from benzodiazepine, a selective serotonin reuptake inhibitor (SSRI), a serotonin-norepinephrine reuptake inhibitor (SNRI), a norepinephrine reuptake inhibitor (NRI), a serotonin 5-hydroxytryptamine1A (5HT1A) antagonist, a dopamine β-hydroxylase inhibitor, an adenosine A2A receptor antagonist, a monoamine oxidase inhibitor (MAOI), a sodium (Na) channel blocker, a calcium channel blocker, a central and peripheral alpha adrenergic receptor antagonist, a central alpha adrenergic agonist, a central or peripheral beta adrenergic receptor antagonist, a NK-1 receptor antagonist, a corticotropin releasing factor (CRF) antagonist, an atypical antidepressant/antipsychotic, a tricyclic, an anticonvulsant, a glutamate antagonist, a gamma-aminobutyric acid (GABA) agonist, and a partial D2 agonist.

22. The method of claim 20, wherein the 4-hydroxy-4-methyl-piperidine-1-carboxylic acid (4-methoxy-7-morpholin-4-yl-benzothiazol-2-yl)-amide reduces at least one of the frequency and intensity of at least one sign of the post-traumatic stress disorder in the patient.

23. The method of claim 20, wherein the 4-hydroxy-4-methyl-piperidine-1-carboxylic acid (4-methoxy-7-morpholin-4-yl-benzothiazol-2-yl)-amide reduces at least one of the frequency and intensity of at least one symptom post-traumatic stress disorder in the patient.

24. A method of diagnosing post-traumatic stress disorder in a patient comprising:
administering to the patient a therapeutically effective amount of 4-hydroxy-4-methyl-piperidine-1-carboxylic acid (4-methoxy-7-morpholin-4-yl-benzothiazol-2-yl)-amide and
assessing at least one of sign, symptom, or symptom cluster of post-traumatic stress disorder; and
diagnosing post-traumatic stress disorder in the patient if the 4-hydroxy-4-methyl-piperidine-1-carboxylic acid (4-methoxy-7-morpholin-4-yl-benzothiazol-2-yl)-amide reduces at least one of sign, symptom, and symptom cluster of post-traumatic stress disorder.

25. The method of claim 24, wherein the patient is a child, adolescent, or adult.
